(19) United States
(12) Patent Application Publication
(10) Pub. No.: US 2004/0157771 A1
(43) Pub. Date: Aug. 12, 2004

(54) Rank-ligand-induced sodium/proton antiporter polypeptides

(76) Inventors: Timothy A. Bird, Bainbridge, WA (US); Mark E. Tometsko, Seattle, WA (US); William C. Dougall, Seattle, WA (US); Bruce A. Mosley, Seattle, WA (US)

Correspondence Address: IMMUNEX CORPORATION, LAW DEPARTMENT, 1201 AMGEN COURT WEST, SEATTLE, WA, 98119, US

(21) Appl. No.: 10/372,613

(22) Filed: Feb. 21, 2003

Related U.S. Application Data
(60) Provisional application No. 60/361,891, filed on Feb. 28, 2002.

(51) Int. Cl. A61K 38/17; C07K 14/705; C07H 21/04

(52) U.S. Cl. 514/12; 530/350; 435/69.1; 435/320.1; 435/325; 536/23.5

(57) ABSTRACT

This invention relates to RIPPA, a new member of the human Na+/H+ antiporter polypeptide family, methods of making such polypeptides, and to methods of using RIPPA and RIPPA-Like polypeptides and agonists or antagonists.

### CROSS-REFERENCE TO RELATED APPLICATIONS

This application hereby claims the benefit of U.S. provisional application serial No. 60/361,891, filed Feb. 28, 2002, the entire disclosure of which is relied upon and incorporated by reference.

### FIELD OF THE INVENTION

This invention relates to human and murine RIPPA polypeptides (RANK-Ligand Induced Proton Pump Analog), new members of a conserved Na+/H+ antiporter polypeptide family; to methods of making RIPPA polypeptides; and to methods of using RIPPA and RIPPA-Like polypeptides and agonists or antagonists.

### BACKGROUND OF THE INVENTION

The Na+/H+ antiporter polypeptides are a related group of transmembrane cation exchangers which use existing electrochemical gradients to move a proton to the extracellular space in exchange for a cation such as sodium ion, lithium ion, or in some instances potassium ion. Na+/H+ antiporters are involved in regulation of the pH and volume of cells and cell compartments, and in responses to osmotic pressure changes. At least one type of Na+/H+ antiporter is expressed in nearly every cell throughout development, however a particular member of the multigene Na+/H+ antiporter polypeptide family can show unique patterns of regulated expression in different cell types and at different stages of development. The biological functions of Na+/H+ antiporters are demonstrated by the phenotypes of knockout mice lacking the function of particular Na+/H+ antiporter gene; such mice have defects in intestinal and kidney sodium ion uptake, or in neural function, or in acid secretion by the gastric mucosa, depending on which member of the multigene Na+/H+ antiporter polypeptide family has been disrupted (Counillon and Pouyssegur, 2000, J Biol Chem 275: 1-4). The myocardial Na+/H+ exchanger has been implicated in myocardial damage during ischemia and reperfusion, with inhibition of the exchanger believed to ameliorate such damage (Karmazyn et al., 1999, Circ Res 85: 777-786).

Common structural features of the Na+/H+ antiporter polypeptides are multiple transmembrane domains; sets of residues that sense pH or interact with sodium ion or another cation; and intracellular domains involved in subcellular localization of the antiporter and/or regulation of antiporter activity. The activities of the Na+/H+ antiporter polypeptide family are mediated through interactions with a variety of molecules, including extracellular and intracellular ions, cytoskeletal components, intracellular kinases, and other intracellular proteins, the latter potentially binding to the cytoplasmic C-terminal domain of Na+/H+ antiporter polypeptides. Characteristics and activities of the Na+/H+ antiporter polypeptide family are described further in Orlowski and Grinstein, 1997, J Biol Chem 272: 22373-22376; Shrode et al., 1998, Am J Physiol 275: C431439; Venturi et al., 2000, J Biol Chem 275: 4734-4742; Wakabayashi et al., 2000, J Biol Chem 275: 7942-794; Murtazina et al., 2001, Eur J Biochem 268: 4674-4685; Numata and Orlowski, 2001, J Biol Chem 276: 17387-17394; and Wiebe et al., 2001, Biochem J 357: 1-109 which are incorporated by reference herein.

In order to develop more effective treatments for conditions and diseases involving cation transport across membranes, such as neural, mucosal, intestinal, renal, cardiac, and immunological conditions, information is needed about previously unidentified members of the Na+/H+ antiporter polypeptide family.

### SUMMARY OF THE INVENTION

The present invention is based upon the discovery of new human and murine Na+/H+ RIPPA polypeptides, new cation antiporter family members.

The invention provides an isolated polypeptide consisting of, consisting essentially of, or more preferably, comprising an amino acid sequence selected from the group consisting of:

(a) the amino acid sequence of SEQ ID NO:2, SEQ ID NO:3, SEQ ID NO:4, or SEQ ID NO:5;

(b) amino acids 113 through 512 of SEQ ID NO:2, amino acids 87 through 455 of SEQ ID NO:3, amino acids 87 through 450 of SEQ ID NO:4, or amino acids 87 through 394 of SEQ ID NO:5;

(c) a fragment of the amino acid sequences of (a) comprising amino acids 514 through 537 of SEQ ID NO:2, wherein a polypeptide consisting of said fragment has cation exchange activity;

(d) amino acids 514 through 537 of SEQ ID NO:2 and an amino acid sequence selected from the group consisting of amino acids 1 through 83; 87 through 105; 107 through 112; 113 through 135; 136 through 138; 139 through 161; 162 through 172; 174 through 191; 193 through 206; 207 through 228; 230 through 233; 234 through 256; 257 through 279; 280 through 302; 303 through 305; 306 through 328; 329 through 340; 342 through 374; 377 through 387; 389 through 411; 412 through 417; 421 through 440; 446 through 451; 453 through 470; or 471 through 489 of SEQ ID NO:2;

(e) amino acids 421 through 440 of SEQ ID NO:2;

(f) an amino acid sequence comprising at least 20 amino acids and sharing amino acid identity with the amino acid sequence of (e), wherein the percent amino acid identity is selected from the group consisting of: at least 75%, at least 80%, at least 85%, at least 90%, at least 95%, at least 97.5%, at least 99%, and at least 99.5%;

(g) an amino acid sequence comprising at least 20 amino acids and sharing amino acid identity with the amino acid sequences of (e), wherein the percent amino acid identity is selected from the group consisting of: at least 75%, at least 80%, at least 85%, at least 90%, at least 95%, at least 97.5%, at least 99%, and at least 99.5%, and wherein a polypeptide comprising said amino acid sequence binds to an antibody that also binds to a polypeptide comprising an amino acid sequence of any of (a)-(d); and

(h) an amino acid sequence of (d)-(g), wherein a polypeptide consisting of said amino acid sequence has cation exchange activity.

The invention further provides an isolated polypeptide consisting of, consisting essentially of, or more preferably, comprising an amino acid sequence selected from the group consisting of:

(a) the amino acid sequence of SEQ ID NO:7;

(b) amino acids 113 through 512 of SEQ ID NO:7;

(c) a fragment of the amino acid sequence of (a) comprising amino acids 514 through 547 of SEQ ID NO:7, wherein a polypeptide consisting of said fragment has cation exchange activity;

(d) amino acids 514 through 547 of SEQ ID NO:7 and an amino acid sequence selected from the group consisting of amino acids 1 through 83; 87 through 105; 107 through 112; 113 through 135; 136 through 138; 139 through 161; 162 through 172; 174 through 191; 193 through 206; 207 through 228; 230 through 233; 234 through 256; 257 through 279; 280 through 302; 303 through 305; 306 through 328; 329 through 340; 342 through 374; 377 through 387; 389 through 411; 412 through 417; 421 through 440; 446 through 451; 453 through 470; or 471 through 489 of SEQ ID NO:7;

(e) amino acids 421 through 440 of SEQ ID NO:7;

(f) an amino acid sequence comprising at least 20 amino acids and sharing amino acid identity with the amino acid sequence of (e), wherein the percent amino acid identity is selected from the group consisting of: at least 75%, at least 80%, at least 85%, at least 90%, at least 95%, at least 97.5%, at least 99%, and at least 99.5%;

(g) an amino acid sequence of comprising at least 20 amino acids and sharing amino acid identity with the amino acid sequences of (e), wherein the percent amino acid identity is selected from the group consisting of: at least 75%, at least 80%, at least 85%, at least 90%, at least 95%, at least 97.5%, at least 99%, and at least 99.5%, and wherein a polypeptide comprising said amino acid sequence binds to an antibody that also binds to a polypeptide comprising an amino acid sequence of any of (a)-(d); and

(h) an amino acid sequence of (d)-(g), wherein a polypeptide consisting of said amino acid sequence has cation exchange activity.

Other aspects of the invention are isolated nucleic acids encoding polypeptides of the invention, with a preferred embodiment being an isolated nucleic acid consisting of, consisting essentially of, or more preferably, comprising a nucleotide sequence selected from the group consisting of:

(a) SEQ ID NO:1;

(b) SEQ ID NO:6;

(c) nucleotides 84 through 1694 of SEQ ID NO:1; and

(d) allelic variants of (a)-(c).

A particular embodiment of the invention is an isolated nucleic acid consisting of, consisting essentially of, or more preferably, comprising a nucleotide sequence selected from the group consisting of 84 through 1694 of SEQ ID NO: 1.

The invention also provides an isolated genomic nucleic acid corresponding to the nucleic acids of the invention.

Other aspects of the invention are isolated nucleic acids encoding polypeptides of the invention, and isolated nucleic acids, preferably having a length of at least 15 nucleotides, that: hybridize under conditions of moderate stringency to the nucleic acids encoding polypeptides of the invention. In preferred embodiments of the invention, such nucleic acids encode a: polypeptide having RIPPA polypeptide activity, or comprise a nucleotide sequence that shares nucleotide sequence identity with the nucleotide sequences of the nucleic acids of the invention, wherein the percent nucleotide sequence identity is selected from the group consisting of: at least 70%, at least 75%, at least 80%, at least 85%, at least 90%, at least 95%, at least 97.5%, at least 99%, and at least 99.5%.

Further provided by the invention are expression vectors and recombinant host cells comprising at least one nucleic acid of the invention, and preferred recombinant host cells wherein said nucleic acid is integrated into the host cell genome.

Also provided is a process for producing a polypeptide encoded by the nucleic acids of the invention, comprising culturing a recombinant host cell under conditions promoting expression of said polypeptide, wherein the recombinant host cell comprises at least one nucleic acid of the invention. A preferred process provided by the invention further comprises purifying said polypeptide. In another aspect of the invention, the polypeptide produced by said process is provided.

Further aspects of the invention are isolated antibodies that bind to the polypeptides of the invention, preferably monoclonal antibodies, also preferably humanized antibodies or humanized antibodies, and preferably wherein the antibody inhibits the activity of said polypeptides.

The invention additionally provides a method of designing an inhibitor of the polypeptides of the invention, the method comprising the steps of determining the three-dimensional structure of any such polypeptide, analyzing the three-dimensional structure for the likely binding sites of substrates, synthesizing a molecule that incorporates a predicted reactive site, and determining the polypeptide-inhibiting activity of the molecule.

In a further aspect of the invention, a method is provided for identifying compounds that alter RIPPA polypeptide activity comprising

(a) mixing a test compound with a polypeptide of the invention; and

(b) determining whether the test compound alters the RIPPA polypeptide activity of said polypeptide.

In another aspect of the invention, a method is provided identifying compounds that inhibit the binding activity of RIPPA polypeptides comprising

(a) mixing a test compound with a polypeptide of the invention and a binding partner of said polypeptide; and

(b) determining whether the test compound inhibits the binding activity of said polypeptide.

The invention also provides a method for increasing cation exchange activities, comprising providing at least one compound selected from the group consisting of the polypeptides of the invention and agonists of said polypeptides; with a preferred embodiment of the method further comprising increasing said activities in a patient by administering at least one polypeptide of the invention.

The above-described methods may be performed in vitro or in vivo. For example, in vitro assays may comprise cell-based assays wherein the cells express one or more forms of RIPPA and/or RIPPA-like polypeptides. Alternative embodiments include assays using a subcellular component of RIPPA and/or RIPPA-like expressing cells, such as membrane preparations expressing RIPPA and/or RIPPA-like polypeptides. Examples of cells that my be used include those of the osteoclast lineage, as well as osteoclast precursor cells and the like.

Further provided by the invention is a method for decreasing cation exchange activity, comprising providing at least one antagonist of the polypeptides of the invention; with a preferred embodiment of the method further comprising decreasing said activities in a patient by administering at least one antagonist of the polypeptides of the invention, and with a further preferred embodiment wherein the antagonist is an antibody that inhibits the activity of any of said polypeptides.

Additional embodiments provide methods of treating diseases or conditions characterized by excessive bone resorption, generally referred to as osteopenias, comprising administering at least one antagonist of RIPPA and/or RIPPA-Like polypeptides. For example, the osteopeniac condition may be selected from the following, but may also include similar conditions not listed herein: osteoporosis, osteomyelitis, hypercalcemia, osteopenia brought on by surgery or steroid administration, prosthetic loosening, Paget's disease, osteonecrosis, bone loss due to rheumatoid arthritis, periodontal bone loss, and cancers that may metastasize to bone and induce bone breakdown, such as multiple myeloma, breast cancer, prostate cancer and some melanomas.

In other aspects of the invention, a method is provided for treating diseases or conditions characterized by a decrease in the rate of bone resportion, generally referred to as osteopetrosis, which is characterized by excessive bone density. The method comprising administering at least one agonist of RIPPA and/or RIPPA-Like polypeptides to a patient suffering from osteopetrosic conditions.

A further embodiment of the invention provides a use for RIPPA and RIPPA-Like polypeptides, as well as agonists and antagonists thereof, in the preparation of a medicament for treating osteopenias or osteopetrosis, wherein the osteopenic condition includes osteoporosis, osteomyelitis, hypercalcemia, osteopenia brought on by surgery or steroid administration, prosthetic loosening, Paget's disease, osteonecrosis, bone loss due to rheumatoid arthritis, periodontal bone loss, and cancers that may metastasize to bone and induce bone breakdown, such as multiple myeloma, breast cancer and some melanomas.

### BRIEF DESCRIPTION OF THE DRAWINGS

FIG. 1 represents a RT-PCR-based assay performed on cDNA extracted from the mouse macrophage cell line RAW 264.7 post stimulation with muRANKL or TNFα. These data show that expression of RIPPA is strongly upregulated in the osteoclast precursor cell line after stimulation with RANKL.

FIG. 2 illustrates the results from a RT-PCR-based assay performed on cDNA extracted from primary monocyte cultures post stimulation with M-CSF (Macrophage Colony Stimulating Factor) and muRANKL or TNFα. These data show that expression of RIPPA is strongly upregulated in the primary monocyte cultures after stimulation with M-CSF/RANKL and M-CSF/TNFα.

### DETAILED DESCRIPTION OF THE INVENTION

Similarities of RIPPA Structure to Other Na+/H+ Antiporter Family Members

We have identified human and murine RIPPA, new Na+/H+ antiporter polypeptides having structural features characteristic of known bacterial Na+/H+ antiporters; the amino acid sequence of human and murine RIPPA polypeptides are provided in SEQ ID NOs 2 and 7, respectively. An alignment showing the sequence similarities between these RIPPA polypeptides and other Na+/H+ antiporter polypeptides is presented in Table 1 in Example 1 below.

One family of Na+/H+ antiporters that has been identified includes the multigenic mammalian NHE (Na+/H+ Exchanger) polypeptide family. At least seven members of this family—NHE1 through NHE7—have been identified so far (Numata and Orlowski, 2001, J Biol Chem 276: 17387-17394). Most of these mammalian Na+/H+ antiporters are located in the plasma membrane, but some members of the family are detected in the Golgi apparatus or in mitochondria. The mammalian NHE family of polypeptides shows sequence similarity with polypeptides from many different metazoans, yeasts, and plants. Structural elements common to NHE Na+/H+ antiporters are ten or twelve transmembrane (TM) domains, N-and/or O-linked glycosylation sites in an extracellular region toward the N-terminal region of the polypeptide, and a cytoplasmic C-terminal region often containing phosphorylation sites, binding sites for regulatory proteins, and binding sites for proteins that are associated with cytoskeletal proteins such as actin filaments. The cation transport activity of NUE antiporters is sensitive to pH, and in the E. coli NhaA Na+/H+ antiporter, regulation of activity by pH is accompanied by a conformational change that exposes the N-terminus of NhaA to the extracellular environment (Venturi et al., 2000, J Biol Chem 275: 4734-4742). However, deletion of the mammalian NHE1 N-terminal region, including the first TM domain and the glycosylation sites, did not substantially affect cation transport (Shrode et al., 1998, Am J Physiol 275: C431-439). The further C-terminal TM domains of NHE Na+/H+ antiporters contain conserved charged residues that are believed to be involved in cation transport and selectivity (Wiebe et al., 2001, Biochem J 357: 1-109). In addition, the TM domains have been implicated in NHE polypeptide homodimer formation.

We have identified a family of Na+/H+ antiporter polypeptides exhibiting the same organization of functional domains as the NHE Na+/H+ antiporter family, but with distinct primary amino acid sequence. This new conserved Na+/H+ antiporter family is therefore considered to be an evolutionary lineage that likely shares the same ancient roots with the NHE Na+/H+ antiporter family, but has developed in parallel to the NHE Na+/H+ antiporters. Representative members of the ‘Non-NHE’ Na+/H+ antiporter family that we have identified are listed in the table below. Drosophila melanogaster and Caenorhabditis elegans each have two Na+/H+ antiporters that are approximately the same in amino acid sequence similarity when compared to the mammalian members of this family; in the mammals the two antiporter genes have diverged into two distinct subfamilies—the ‘RIPPA’ group and the ‘RIPPA-Like’ group. The first Homo sapiens entries in the table below—RIPPA (SEQ ID NO:2) and GeneSeq AAY94918—are the longest human versions of each subfamily; the remaining human entries appear to be various truncated and/or alternative splice forms of RIPPA or GeneSeq AAY94918. The RIPPA and RIPPA-Like Na+/H+ antiporter polypeptide family is extremely conserved, with the human RIPPA and RIPPA-Like family members highly similar to each other, and even to Na+/H+ antiporter family members from prokaryotes.

Conserved Na+/H+ Antiporter Family which is Distinct from NHE Na+/H+ Antiporter Family

| RIPPA Na+/H+ Antiporters | | RIPPA-Like Na+/H+ Antiporters | |
|---|---|---|---|
| Species | Accession Number(s) | Species | Accession Number(s) |
| H. sapiens | RIPPA (SEQ ID NOs 2, 3, 4, 5) | H. sapiens | GeneSeq AAY94918 |
| H. sapiens | GenBank XP_059638 | H. sapiens | GeneSeq AAU01672; GeneSeq |
| | | | AAU01642 |
| H. sapiens | SWISSPROT/trEMBL Q96D95; | H. sapiens | GenBank XP_058791; GenBank |
| | GenBank AAH09732, BC009732 | | XP_059639; GenBank XP_067128; |
| | | | GenBank XP_058331 |
| H. sapiens | GeneSeq AAM00971; GeneSeq | H. sapiens | GeneSeq AAM65133; GeneSeq |
| | AAM25294; GeneSeq AAO11623; | | AAM38060; GenBank XP_065517 |
| | GeneSeq AAM00858; GeneSeq | | |
| | AAB63156; GeneSeq AAW78172; | | |
| | GeneSeq AAW78301; GeneSeq | | |
| | AAW78302 | | |
| | | Macaca | SWISSPROT/trEMBL Q95JS4; |
| | | fascicularis | GenBank BAB63050, AB070105 |
| Mus | RIPPA (SEQ ID NO: 7) | Mus | SWISSPROT/trEMBL Q9D400; |
| musculus | | musculus | GenBank BAB30495, AK016917 |

| Species | Accession Number(s) |
|---|---|
| Drosophila | GenBank AAL13583, AY058354 and GenBank AAF52449, AE003615^{‡} |
| melanogaster | GenBank AAL13541, AY058312 |
| Caenorhabditis elegans | SWISSPROT/trEMBL Q20273, GenBank T22074 |
| | SWISSPROT/trEMBL Q9XU88, GenBank T22876 |
| Methanothermobacter | SWISSPROT/trEMBL O26854, GenBank NP_275902, NC_000916 |
| thermautotrophicus | |
| Nostoc sp. | GenBank NP_486304.1, NC_003272 |
| Clostridium difficile | SWISSPROT/trEMBL P97213; GenBank JC5342, CAA63558, X92982 |

^{‡}GenBank AAL13583 is an alternate splice form or truncated version of GenBank AAF52449.

The typical structural elements common to members of the RIPPA and RIPPA-Like Na+/H+ antiporters polypeptide families include multiple transmembrane domains. The locations of the transmembrane domains in RIPPA polypeptides are shown graphically below and by amino acid sequence location in Tables 1 and 2, respectively, of Example 1 below. The C-terminal cytoplasmic domain extends from approximately amino acid 513 or 514 of SEQ ID NO:2 and extends through the carboxyl terminus of the polypeptide (amino acid 537 of SEQ ID NO:2). The C-terminal valine residue and nearby serine residue (amino acid 534 of SEQ ID NO:2) of human RIPPA polypeptides are similar to known C-terminal binding sequences for PDZ-domain containing proteins; the NEE family Na+/H+ antiporter NHE3 is regulated through interactions between its cytoplasmic C-terminal domain and PDZ-domain-containing proteins (Yun et al., 1998, J Biol Chem 273: 25856-25863). Evolutionarily conserved charged residues are found within or at the boundaries of TM domains within human and murine RIPPA—notably the arginine residues (amino acids 177 and 187 of SEQ ID NO:2) within TM3; the pair of Asp residues (amino acids 278 and 279 of SEQ ID NO:2) at the edge of TM6; the arginine residues (amino acids 330 and 432 of SEQ ID NO:2) at the edge of TM7 and within TM10, respectively; the lysine residues (amino acids 448, 450, and 460 of SEQ ID NO:2) at the edge and within TM11; and the arginine residue (amino acid 515 of SEQ ID NO:2) at the edge of TM12. Therefore, RIPPA Na+/H+ antiporter polypeptides have an overall multi-TM structure consistent with other Na+/H+ antiporter polypeptides, and include highly conserved residues that are also consistent with Na+/H+ antiporter function.

The skilled artisan will recognize that the boundaries of the regions of RIPPA polypeptides described above are approximate and that the precise boundaries of such domains, as for example the boundaries of the transmembrane region (which can be predicted by using computer programs available for that purpose), can also differ from member to member within the Na+/H+ antiporter polypeptide family.

Biological Activities and Functions of RIPPA Polypeptides

RIPPA (RANKL-Induced Proton Pump Analog) nucleic acid and polypeptide sequences were identified following the discovery that RIPPA expression was strongly increased in a murine macrophage cell line (RAW 264.7) after exposure to RANK Ligand (RANKL). It is known that the RAW 264.7 macrophage cell line differentiates to a mature osteoclast phenotype if proper stimulatory signals are provided. Macrophage colony-stimulating factor (M-CSF) and RANKL have been shown to be essential and sufficient to induce maturation of macrophages into osteoclasts (see, Teitelbaum, S. L., et al., 2000, Science, 289:1504). RANKL stimulates the M-CSF-expanded precursors to commit to the osteoclast phenotype.

RANK (Receptor Activator of NF-κB) and its ligand (RANKL) are a receptor/ligand pair that play an important role in immune responses and in bone metabolism. RANK and RANKL, both murine and human, have been cloned and characterized (see, for example, U.S. Pat. No. 6,017,729, WO 98/25958, EP 0 873 998, EP 0 911 342, U.S. Pat. No. 5,843,678, WO 98/46751 and WO 98/54201). RANKL has also been called “osteoprotegerin binding protein,” “osteoclastogenesis differentiation factor,” and “TRANCE” (see, for example, Kodaira et al., 1999; Yasuda et al., Proc. Natl. Acad. Sci. 95:3597 (1998); and Wong et al., J Biol Chem 273(43):28355-59 (1998)). RANKL binds not only to RANK, but also to a naturally occurring RANK decoy protein called osteoprotegerin (OPG), which is a member of the tumor necrosis factor receptor family (see, for example, U.S. Pat. No. 6,015,938 and WO 98/46751). OPG is a soluble molecule whose role in bone metabolism is reviewed in Hofbauer et al., J Bone Min Res 15(1):2-12 (2000). Further aspects of RANK/RANKL and OPG biology are discussed, for example, in Simonet et al., Cell 89:309-319 (1997); Kodaira et al., Gene 230:121-27 (1999); U.S. Pat. No. 5,843,678; and U.S. Pat. No. 6,015,938.

Terminal differentiation of haematopoietic cells of the monocyte/mcarophage lineage eventually leads to active, multinucleated bone-resorbing osteoclasts. Osteoclasts resorb mineralized tissues after a series of cellular polarization events, such as the cytoskeletal formation of podosomes that enclose a specialzed secretory membrane—the ruffled membrane. The ruffled membrane is thought to represent subcellular accumulation of acidifying vesicles along microtubules and polarized insertion into the plasma membrane. Bone demineralization involves intimate contact with the bone matrix and acidification of the isolated extracellular microenvironment, a process mediated by a vacuolar H+-adenosine triphosphatase (H+-ATPase) in the cell's ruffled membrane. The intra-osteoclastic pH is maintained by an energy-independent Cl-/HCO3-exchanger on the cell's antiresorptive surface. Additionally, electroneutrality is preserved by a ruffled membrane Cl-channel, charge-coupled to the H+-ATPase. The result of these ion transporting events is secretion of HCl, creating a pH of ˜4.5 in the resorptive microenvironment. After acidification and demineralization of the bone, the organic component of bone is degraded by cathepsin K, a lysosomal protease. The products of bone degradation are endocytosed by the osteoclast and transported to and released at the cell's antiresorptive surface (see Teitelbaum, S. L., et al., 2000; Science, 289:1504).

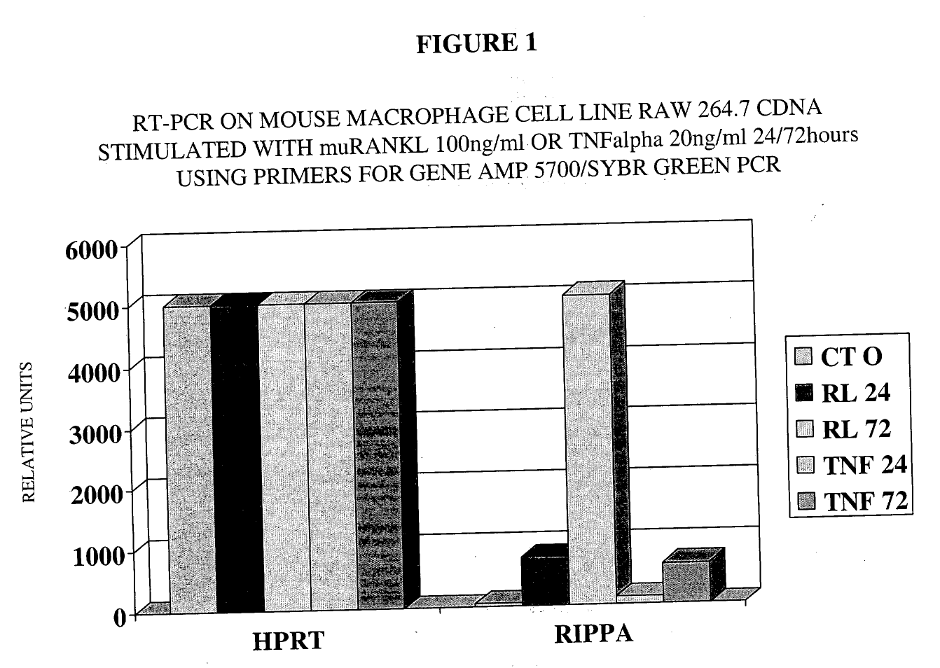

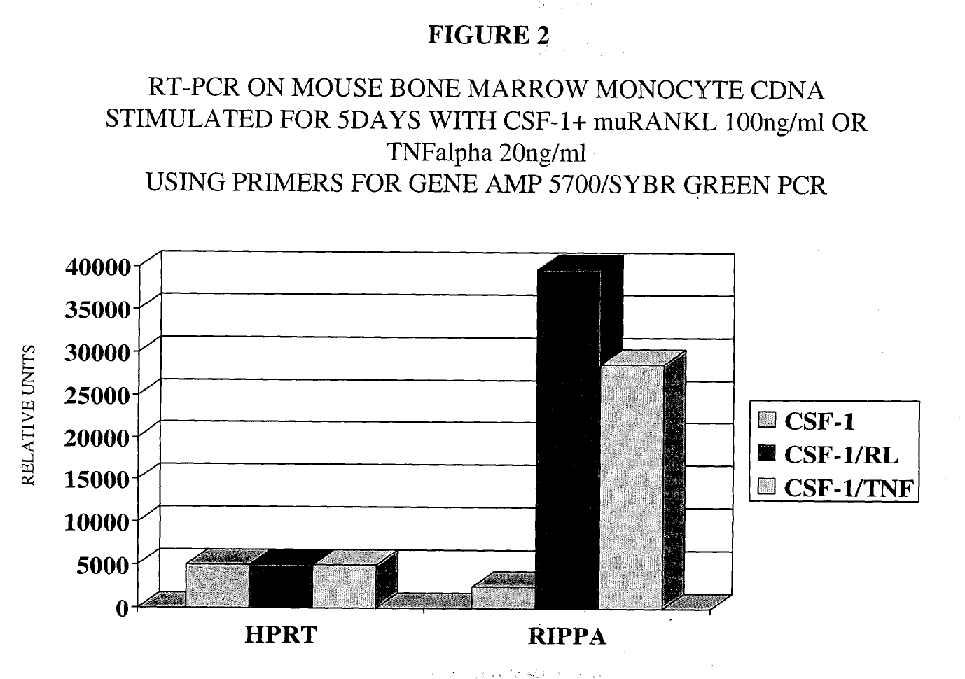

To further elucidate the relationship between RIPPA expression and RANKL-induced osteoclastogenesis, a series of studies were performed as described in Examples 4 and 5. In summary, real-time PCR analysis of RIPPA cDNA levels in RAW 264.7 cells exposed to RANKL or TNFα showed tremendous upregulation of RIPPA in response to RANKL, but comparatively little upregulation of RIPPA in response to TNFα (see Example 4 and FIG. 1). Real-time PCR analysis of RIPPA cDNA levels in bone marrow-drived primary monocyte cultures stiumulated for 5 days with M-CSF (also referred to as CSF-1) and RANKL or M-CSF and TNFα showed surprising upregualtion of RIPPA in response to M-CSF/RANKL, as well as upregulation of RIPPA in response to M-CSF/TNFα, but not to M-CSF alone (see Example 5 and FIG. 2). Together these studies show that RIPPA is upregulated in response to RANKL, which is known to induce differentiation of monocytes/macrophages into mature osteoclasts. Therefore, RIPPA and RIPPA-Like polypeptides are involved in osteoclastogenesis and/or osteoclastic bone resorption processes.

Because RIPPA and RIPPA-Like polypeptides are linked to osteoclastogenesis and/or osteoclastic bone resorption processes, RIPPA and RIPPA-Like polypeptides are likely implicated in diseases or conditions characterized by excessive bone resorption, generally referred to as osteopenias. Therefore, methods are provided for treating such disorders by administering antagonists or agonists of RIPPA and RIPPA-Like polypeptides, as well as antagonists or agonists to their substrates, ligands, receptors, binding partners, and or other interacting polypeptides. Exemplary osteopenic conditions that may be treated with RIPPA and RIPPA-Like antagonists include, but are not limited to: osteoporosis, osteomyelitis, hypercalcemia, osteopenia brought on by surgery or steroid administration, prosthetic loosening, Paget's disease, osteonecrosis, bone loss due to rheumatoid arthritis, periodontal bone loss, and cancers that may metastasize to bone and induce bone breakdown.

With regards to cancer, some investigators have observed that certain cancer cells secrete a soluble form of RANKL that appears to contribute to hypercalcemia or to the establishment of malignant bone lesions (Nagai et al., Biochem Biophys Res Comm 269:532-536 (2000); and Zhang et al., 2001). Overproduction of parathyroid hormone-related protein also is believed to contribute to the hypercalcemia of cancer (see, for example, Rankin et al., Cancer (Suppl) 80(8):1564-71 (1997)). Hypercalcemia, a late complication of cancer, disrupts the body's ability to maintain a normal level of calcium, and can result in fatigue, calcium deposits in the kidneys, heart problems and neural dysfunction. Hypercalcemia occurs most frequently in patients with lung and breast cancer, and also is known to occur in patients with multiple myeloma, head and neck cancer, sarcoma, cancer of unknown primary origin, lymphoma, leukemia, melanoma, kidney cancer, and the gastrointestinal cancers, which includes esophageal, stomach, intestinal, colon and rectal cancers. As mentioned above, embodiments of the present invention are drawn to methods of treating hypercalcaemia by administering RIPPA and RIPPA-Like polypeptides and/or antagonists or agonists of RIPPA and RIPPA-Like polypeptides, as well as antagonists or agonists to their substrates, ligands, receptors, binding partners, and or other interacting polypeptides.

Conversely, RIPPA and RIPPA-Like polypeptides may also be implicated in diseases or conditions characterized by a decrease in the rate of bone resportion, generally referred to as osteopetrosis, which is characterized by excessive bone density. Alternative embodiments are drawn to methods of treating osteopetrosis by administering RIPPA and RIPPA-Like polypeptides and/or agonists or antagonists of RIPPA and RIPPA-Like polypeptides, as well as agonists or antagonists to their substrates, ligands, receptors, binding partners, and or other interacting polypeptides.

To further characterize RIPPA and RIPPA-Like molecules, PCR amplification from tissue-specific cDNA libraries was performed to detect human RIPPA cDNA sequences. The results of these experiments show that human RIPPA transcripts are expressed in a wide variety of fetal cells and adult cells, but not in placenta or skeletal muscle. On the other hand, the RIPPA-Like sequence GeneSeq AAM38060 appears to be expressed in placental tissue. Thus, a combination of nucleic acid or antibody probes designed using human RIPPA sequences and GeneSeq AAM38060 sequences can be used to provide a specific and reliable diagnostic for detecting placental tissue: the human RIPPA probe will react weakly or not at all with the suspected placental sample, and the GeneSeq AAM38060 probe will react with the placental tissue sample.

Further embodiments are drawn to treating conditions and diseases that share cation exchange disregulation as a common feature in their etiology. For example, NEE antiporters have been implicated in a number of pathological conditions, such as chronic metabolic acidosis and alkalosis; myocardial, cerebral and renal ischaemic and reperfusion pathology; aberrant cerebral functioning including abnormal memory and cognitive functions; congenital sodium diarrhea; gastrointestinal pathologies; coronary artery diseases, such as acute responses to coronary occlusion; chronic hypertension; renal disease; diabetes and diabetes-induced vascular hypertrophy; epilepsy; cancers, such as gliomas; and, gial and astrogial pathologies. Thus, antagonists or agonists of the RIPPA and RIPPA-Like polypeptides described herein may be used in methods of treating patients suffering from such disorders.

Blocking or inhibiting the interactions between members of the RIPPA and RIPPA-Like polypeptide family and their substrates, ligands, receptors, binding partners, and or other interacting polypeptides is an aspect of the invention and provides methods for treating or ameliorating these diseases and conditions through the use of inhibitors of RIPPA and/or and RIPPA-Like polypeptide activity. Examples of such inhibitors or antagonists are described in more detail below. For certain conditions involving too little RIPPA or and RIPPA-Like polypeptide activity, methods of treating or ameliorating these conditions comprise increasing the amount or activity of RIPPA or and RIPPA-Like polypeptides by providing isolated RIPPA or and RIPPA-Like polypeptides or active fragments or fusion polypeptides thereof, or by providing compounds (agonists) that activate endogenous or exogenous RIPPA and/or and RIPPA-Like polypeptides. Preferred methods of administering RIPPA and and RIPPA-Like polypeptides to organisms in need of treatment, such as mammals or most preferably humans, include in vivo or ex vivo treament of cells with viral particles or liposomes containing nucleic acids encoding RIPPA and/or and RIPPA-Like polypeptides to be expressed in target cells of the organism in need of treatment.

In certain embodiments, typical biological activities or functions associated with RIPPA and RIPPA-Like polypeptides involve cation transport. RIPPA and RIPPA-Like polypeptides having cation exchange activity transport cations through a cellular membrane in exchange for protons. The cation exchange activity is associated with the TM domains of RIPPA polypeptides. Thus, for uses requiring cation exchange activity, preferred RIPPA polypeptides include those having the TM domains and exhibiting cation exchange biological activity. Preferred RIPPA polypeptides further include oligomers or fusion polypeptides comprising at least one TM domain portion of one or more RIPPA or RIPPA-Like polypeptides, and fragments of any of these polypeptides that have cation exchange activity. The cation exchange activity of RIPPA polypeptides can be determined, for example, in an assay that measures cation transport in repsonse to cellular stimuli, or proton-dependent cation transport. Polypeptides having cation exchange activity preferably have at least 10% (more preferably, at least 25%, and most preferably, at least 50%) of the cation exchange activity of NHE7 as measured in FIG. 7 of Numata and Orlowski, 2001, J Biol Chem 276: 17387-17394.

The term “RIPPA polypeptide activity,” as used herein, includes any one or more of the following: osteoclastogenesis, bone resorption processes, cation exchange activity as well as the ex vivo and in vivo activities of RIPPA and RIPPA-Like family polypeptides. The degree to which individual members of the RIPPA polypeptide family and fragments and other derivatives of these polypeptides exhibit these activities can be determined by standard assay methods. Exemplary assays are disclosed herein; those of skill in the art will appreciate that other, similar types of assays can be used to measure RIPPA family biological activities.

Another aspect of the biological activity of RIPPA polypeptides is the ability of members of this polypeptide family to bind particular binding partners such as PDZ-domain-containing polypeptides, kinases, and cytoskeletal or cytoskeleton-associated polypeptides, with the cytoplasmic C-terminal domain of RIPPA polypeptides binding to such polypeptides. The term “binding partner,” as used herein, includes ligands, receptors, substrates, antibodies, other RIPPA and RIPPA-Like polypeptides, the same RIPPA or RIPPA-Like polypeptide (in the case of homotypic interactions), and any other molecule that interacts with a RIPPA or RIPPA-Like polypeptide through contact or proximity between particular portions of the binding partner and the RIPPA or RIPPA-Like polypeptide.

Because the cytoplasmic C-terminal domain of RIPPA polypeptides is predicted to bind to binding partners, the cytoplasmic C-terminal domain when expressed as a separate fragment from the rest of a RIPPA polypeptide is expected to disrupt the binding of RIPPA polypeptides to such intracellular binding partners. By binding to one or more binding partners, the separate cytoplasmic C-terminal domain polypeptide likely prevents binding by the native RIPPA polypeptide(s), and so acts in a dominant negative fashion to inhibit the biological activities mediated via binding of RIPPA polypeptides to binding partners. Suitable assays to detect or measure the binding between RIPPA polypeptides and their binding partners are yeast two-hybrid assays and other methods disclosed herein.

RIPPA and RIPPA-Like Polypeptides

A RIPPA polypeptide is a polypeptide that shares a sufficient degree of amino acid identity or similarity to the RIPPA polypeptides of SEQ ID NOs 2 through 5 and 7 to (A) be identified by those of skill in the art as a polypeptide likely to share particular structural domains and/or (B) have biological activities in common with the RIPPA polypeptide of SEQ ID NOs 2 through 5 and 7 and/or (C) bind to antibodies that also specifically bind to other RIPPA polypeptides. RIPPA and RIPPA-Like polypeptides can be isolated from naturally occurring sources, or have the same structure as naturally occurring RIPPA or RIPPA-Like polypeptides, or can be produced to have structures that differ from naturally occurring RIPPA or RIPPA-Like polypeptides. Polypeptides derived from any RIPPA or RIPPA-Like polypeptide by any type of alteration (for example, but not limited to, insertions, deletions, or substitutions of amino acids; changes in the state of glycosylation of the polypeptide; refolding or isomerization to change its three-dimensional structure or self-association state; and changes to its association with other polypeptides or molecules) are also RIPPA or RIPPA-Like polypeptides. Therefore, the polypeptides provided by the invention include polypeptides characterized by amino acid sequences similar to those of the RIPPA and RIPPA-Like polypeptides described herein, but into which modifications are naturally provided or deliberately engineered. A polypeptide that shares biological activities in common with RIPPA or RIPPA-Like polypeptides is a polypeptide having RIPPA polypeptide activity. Examples of biological activities exhibited by RIPPA polypeptides include, without limitation, osteoclastogenesis, bone resorption processes, cation exchange activity and the like.

The present invention provides both full-length and mature forms of RIPPA polypeptides. Full-length polypeptides are those having the complete primary amino acid sequence of the polypeptide; as initially translated. The amino acid sequences of full-length polypeptides can be obtained, for example, by translation of the complete open reading frame (“ORF”) of a cDNA molecule. Several full-length polypeptides can be encoded by a single genetic locus if multiple mRNA forms are produced from that locus by alternative splicing or by the use of multiple translation initiation sites. The “mature form” of a polypeptide refers to a polypeptide that has undergone post-translational processing steps such as cleavage of the signal sequence or proteolytic cleavage to remove a prodomain. Multiple mature forms of a particular full-length polypeptide may be produced, for example by cleavage of the signal sequence at multiple sites, or by differential regulation of proteases that cleave the polypeptide. A polypeptide preparation can therefore include a mixture of polypeptide molecules having different N-terminal amino acids. The mature form(s) of such polypeptide can be obtained by expression, in a suitable mammalian cell or other host cell, of a nucleic acid molecule that encodes the full-length polypeptide. Also encompassed within the invention are variations attributable to differences in proteolysis in different types of host cells, such as differences in the position of cleavage of the signal peptide, or differences in the N- or C-termnini due to proteolytic removal of one or more terminal amino acids from the polypeptide (generally from 1-5 terminal amino acids). The sequence of the mature form of the polypeptide may be determinable from the amino acid sequence of the full-length form, through identification of signal sequences or protease cleavage sites. The RIPPA polypeptides of the invention also include those that result from post-transcriptional or post-translational processing events such as alternate mRNA processing which can yield a truncated but biologically active polypeptide, for example, a naturally occurring soluble form of the polypeptide.

The invention further includes RIPPA polypeptides with or without associated native-pattern glycosylation. Polypeptides expressed in yeast or mammalian expression systems (e.g., COS-1 or CHO cells) can be similar to or significantly different from a native polypeptide in molecular weight and glycosylation pattern, depending upon the choice of expression system. Expression of polypeptides of the invention in bacterial expression systems, such as E. coli, provides non-glycosylated molecules. Further, a given preparation can include multiple differentially glycosylated species of the polypeptide. Glycosyl groups can be removed through conventional methods, in particular those utilizing glycopeptidase. In general, glycosylated polypeptides of the invention can be incubated with a molar excess of glycopeptidase (Boehringer Mannheim).

Species homologues of RIPPA and RIPPA-Like polypeptides and of nucleic acids encoding them are also provided by the present invention. As used herein, a “species homologue” is a polypeptide or nucleic acid with a different species of origin from that of a given polypeptide or nucleic acid, but with significant sequence similarity to the given polypeptide or nucleic acid, as determined by those of skill in the art. Species homologues can be isolated and identified by making suitable probes or primers from polynucleotides encoding the amino acid sequences provided herein and screening a suitable nucleic acid source from the desired species. The invention also encompasses allelic variants of RIPPA and RIPPA-Like polypeptides and nucleic acids encoding them; that is, naturally-occurring alternative forms of such polypeptides and nucleic acids in which differences in amino acid or nucleotide sequence are attributable to genetic polymorphism (allelic variation among individuals within a population).

Fragments of the RIPPA and RIPPA-Like polypeptides of the present invention are encompassed by the present invention and can be in linear form or cyclized using known methods, for example, as described in Saragovi et al., Bio/Technology 10, 773-778 (1992) and in McDowell et al., J. Amer. Chem. Soc. 114 9245-9253 (1992). Polypeptides and polypeptide fragments of the present invention, and nucleic acids encoding them, include polypeptides and nucleic acids with amino acid or nucleotide sequence lengths that are at least 25% (at least 50%, or at least 60%, at least 70%, or at least 80%) of the length of a RIPPA or RIPPA-Like polypeptide and have at least 60% sequence identity (at least 70%, at least 75%, at least 80%, at least 85%, at least 90%, at least 95%, at least 97.5%, at least 99%, or at least 99.5%) with that RIPPA or RIPPA-Like polypeptide or encoding nucleic acid, where sequence identity is determined by comparing the amino acid sequences of the polypeptides when aligned so as to maximize overlap and identity while minimizing sequence gaps. Also included in the present invention are polypeptides and polypeptide fragments, and nucleic acids encoding them, that contain or encode a segment preferably comprising at least 8, or at least 10, or preferably at least 15, or at least 20, or at least 30, or at least 40 contiguous amino acids. Such polypeptides and polypeptide fragments may also contain a segment that shares at least 70% sequence identity (at least 70%, at least 75%, at least 80%, at least 85%, at least 90%, at least 95%, at least 97.5%, at least 99%, or at least 99.5%) with any such segment of any RIPPA or RIPPA-Like polypeptide, where sequence identity is determined by comparing the amino acid sequences of the polypeptides when aligned so as to maximize overlap and identity while minimizing sequence gaps. The percent identity of two amino acid or two nucleic acid sequences can be determined by visual inspection and mathematical calculation, or more preferably, the comparison is done by comparing sequence information using a computer program. An exemplary, preferred computer program is the Genetics Computer Group (GCG; Madison, Wis.) Wisconsin package version 10.0 program, ‘GAP’ (Devereux et al., 1984, Nucl. Acids Res. 12: 387). The preferred default parameters for the ‘GAP’ program includes: (1) The GCG implementation of a unary comparison matrix (containing a value of 1 for identities and 0 for non-identities) for nucleotides, and the weighted amino acid comparison matrix of Gribskov and Burgess, Nucl. Acids Res. 14:6745, 1986, as described by Schwartz and Dayhoff, eds., Atlas of Polypeptide Sequence and Structure, National Biomedical Research Foundation, pp: 353-358, 1979; or other comparable comparison matrices; (2) a penalty of 30 for each gap and an additional penalty of 1 for each symbol in each gap for amino acid sequences, or penalty of 50 for each gap and an additional penalty of 3 for each symbol in each gap for nucleotide sequences; (3) no penalty for end gaps; and (4) no maximum penalty for long gaps. Other programs used by those skilled in the art of sequence comparison can also be used, such as, for example, the BLASTN program version 2.0.9, available for use via the National Library of Medicine website ncbi.nlm.nih.gov/gorf/wblast2.cgi, or the UW-BLAST 2.0 algorithm. Standard default parameter settings for UW-BLAST 2.0 are described at the following Internet site: sapiens.wustl.edufblast/blast/#Features. In addition, the BLAST algorithm uses the BLOSUM62 amino acid scoring matix, and optional parameters that can be used are as follows: (A) inclusion of a filter to mask segments of the query sequence that have low compositional complexity (as determined by the SEG program of Wootton and Federhen (Computers and Chemistry, 1993); also see Wootton and Federhen, 1996, Analysis of compositionally biased regions in sequence databases, Methods Enzymol. 266: 554-71) or segments consisting of short-periodicity internal repeats (as determined by the XNU program of Claverie and States (Computers and Chemistry, 1993)), and (B) a statistical significance threshold for reporting matches against database sequences, or E-score (the expected probability of matches being found merely by chance, according to the stochastic model of Karlin and Altschul (1990); if the statistical significance ascribed to a match is greater than this E-score threshold, the match will not be reported.); example E-score threshold values are 0.5, 0.25, 0.1, 0.05, 0.01, 0.001, 0.0001, 1e-5, 1e-10, 1e-15, 1e-20, 1e-25, 1e-30, 1e40, 1e-50, 1e-75, or 1e-100.

The present invention also provides for soluble forms of RIPPA polypeptides comprising certain fragments or domains of these polypeptides, and particularly those comprising an intracellular domain, an extracellular domain, or one or more fragments of an intracellular or extracellular domain. Soluble polypeptides are polypeptides that are capable of being secreted from the cells in which they are expressed. In such forms part or all of the transmembrane domains of the polypeptide are deleted such that the polypeptide is fully secreted from the cell in which it is expressed. The extracellular, intracellular, and transmembrane domains of polypeptides of the invention can be identified in accordance with known techniques for determination of such domains from sequence information. Soluble RIPPA and RIPPA-Like polypeptides also include those polypeptides which include part of the transmembrane region, provided that the soluble RIPPA or RIPPA-Like polypeptide is capable of being secreted from a cell, and preferably retains RIPPA polypeptide activity. Soluble RIPPA and RIPPA-Like polypeptides further include oligomers or fusion polypeptides comprising the extracellular or intracellular portion of at least one RIPPA or RIPPA-Like polypeptide, and fragments of any of these polypeptides that have RIPPA polypeptide activity. A secreted soluble polypeptide can be identified (and distinguished from its non-soluble membrane-bound counterparts) by separating intact cells which express the desired polypeptide from the culture medium, e.g., by centrifugation, and assaying the medium (supernatant) for the presence of the desired polypeptide. The presence of the desired polypeptide in the medium indicates that the polypeptide was secreted from the cells and thus is a soluble form of the polypeptide. The use of soluble forms of RIPPA polypeptides is advantageous for many applications. Purification of the polypeptides from recombinant host cells is facilitated, since the soluble polypeptides are secreted from the cells. Moreover, soluble polypeptides are generally more suitable than membrane-bound forms for parenteral administration and for many enzymatic procedures.

In another aspect of the invention, preferred polypeptides comprise various combinations of RIPPA and/or RIPPA-Like polypeptide domains, such as one or more TM domains and the cytoplasmic C-terminal domain. Accordingly, polypeptides of the present invention and nucleic acids encoding them include those comprising or encoding two or more copies of a domain such one or more TM domains, two or more copies of a domain such as the cytoplasmic C-terminal domain, or at least one copy of each domain, and these domains can be presented in any order within such polypeptides.

Further modifications in the peptide or DNA sequences can be made by those skilled in the art using known techniques. Modifications of interest in the polypeptide sequences can include the alteration, substitution, replacement, insertion or deletion of a selected amino acid. For example, one or more of the cysteine residues can be deleted or replaced with another amino acid to alter the conformation of the molecule, an alteration which may involve preventing formation of incorrect intramolecular disulfide bridges upon folding or renaturation. Techniques for such alteration, substitution, replacement, insertion or deletion are well known to those skilled in the art (see, e.g., U.S. Pat. No. 4,518,584). As another example, N-glycosylation sites in the polypeptide extracellular domain can be modified to preclude glycosylation, allowing expression of a reduced carbohydrate analog in mammalian and yeast expression systems. N-glycosylation sites in eukaryotic polypeptides are characterized by an amino acid triplet Asn-X—Y, wherein X is any amino acid except Pro and Y is Ser or Thr. Appropriate substitutions, additions, or deletions to the nucleotide sequence encoding these triplets will result in prevention of attachment of carbohydrate residues at the Asn side chain. Alteration of a single nucleotide, chosen so that Asn is replaced by a different amino acid, for example, is sufficient to inactivate an N-glycosylation site. Alternatively, the Ser or Thr can by replaced with another amino acid, such as Ala. Known procedures for inactivating N-glycosylation sites in polypeptides include those described in U.S. Pat. No. 5,071,972 and EP 276,846. Additional variants within the scope of the invention include polypeptides that can be modified to create derivatives thereof by forming covalent or aggregative conjugates with other chemical moieties, such as glycosyl groups, lipids, phosphate, acetyl groups and the like. Covalent derivatives can be prepared by linking the chemical moieties to functional groups on amino acid side chains or at the N-terminus or C-terminus of a polypeptide. Conjugates comprising diagnostic (detectable) or therapeutic agents attached thereto are contemplated herein. Preferably, such alteration, substitution, replacement, insertion or deletion retains the desired activity of the polypeptide or a substantial equivalent thereof. One example is a variant that binds with essentially the same binding affinity as does the native form. Binding affinity can be measured by conventional procedures, e.g., as described in U.S. Pat. No. 5,512,457 and as set forth herein.

Other derivatives include covalent or aggregative conjugates of the polypeptides with other polypeptides or polypeptides, such as by synthesis in recombinant culture as N-terminal or C-terminal fusions. Examples of fusion polypeptides are discussed below in connection with oligomers. Further, fusion polypeptides can comprise peptides added to facilitate purification and identification. Such peptides include, for example, poly-His or the antigenic identification peptides described in U.S. Pat. No. 5,011,912 and in Hopp et al., Bio/Technology 6:1204, 1988. One such peptide is the FLAG® peptide, which is highly antigenic and provides an epitope reversibly bound by a specific monoclonal antibody, enabling rapid assay and facile purification of expressed recombinant polypeptide. A murine hybridoma designated 4E11 produces a monoclonal antibody that binds the FLAG® peptide in the presence of certain divalent metal cations, as described in U.S. Pat. No. 5,011,912. The 4E11 hybridoma cell line has been deposited with the American Type Culture Collection under accession no. HB 9259. Monoclonal antibodies that bind the FLAG“peptide are available from Eastman Kodak Co., Scientific Imaging Systems Division, New Haven, Conn.

Encompassed by the invention are oligomers or fusion polypeptides that contain a RIPPA or RIPPA-Like polypeptide, one or more fragments of RIPPA or RIPPA-Like polypeptides, or any of the derivative or variant forms of RIPPA and RIPPA-Like polypeptides as disclosed herein. In particular embodiments, the oligomers comprise soluble RIPPA or RIPPA-Like polypeptides. Oligomers can be in the form of covalently linked or non-covalently-linked multimers, including dimers, trimers, or higher oligomers. In one aspect of the invention, the oligomers maintain the binding ability of the polypeptide components and provide therefor, bivalent, trivalent, etc., binding sites. In an alternative embodiment the invention is directed to oligomers comprising multiple RIPPA and/or RIPPA-Like polypeptides joined via covalent or non-covalent interactions between peptide moieties fused to the polypeptides, such peptides; having the; property of promoting oligomerization. Leucine zippers and certain polypeptides derived from antibodies are among the peptides that can promote oligomerization of the polypeptides attached thereto, as described in more detail below.

Membrane-spanning RIPPA or RIPPA-Like polypeptides can be fused with extracellular or intracellular domains of receptor polypeptides for which the ligand is known. Such fusion polypeptides can then be manipulated to control the intracellular signaling pathways triggered by the membrane-spanning RIPPA or RIPPA-Like polypeptide. RIPPA and RIPPA-Like polypeptides that span the cell membrane can also be fused with agonists or antagonists of cell-surface receptors, or cellular adhesion molecules to further modulate RIPPA intracellular effects. In another aspect of the present invention, interleukins can be situated between the preferred RIPPA or RIPPA-Like polypeptide fragment and other fusion polypeptide domains.

Immunoglobulin-based Oligomers. The polypeptides of the invention or fragments thereof can be fused to molecules such as immunoglobulins for many purposes, including increasing the valency of polypeptide binding sites. For example, fragments of a RIPPA or RIPPA-Like polypeptide can be fused directly or through linker sequences to the Fc portion of an immunoglobulin. For a bivalent form of the polypeptide, such a fusion could be to the Fc portion of an IgG molecule. Other immunoglobulin isotypes can also be used to generate such fusions. For example, a polypeptide-IgM fusion would generate a decavalent form of the polypeptide of the invention. The term “Fe polypeptide” as used herein includes native and mutein forms of polypeptides made up of the Fe region of an antibody comprising any or all of the CH domains of the Fe region. Truncated forms of such polypeptides containing the hinge region that promotes dimerization are also included. Preferred Fe polypeptides comprise an Fe polypeptide derived from a human IgG1 antibody. As one alternative, an oligomer is prepared using polypeptides derived from immunoglobulins. Preparation of fusion polypeptides comprising certain heterologous polypeptides fused to various portions of antibody-derived polypeptides (including the Fc domain) has been described, e.g., by Ashkenazi et al. (PNAS USA 88:10535, 1991); Byrn et al. (Nature 344:677, 1990); and Hollenbaugh and Aruffo (“Construction of Immunoglobulin Fusion Polypeptides”, in Current Protocols in Immunology, Suppl. 4, pages 10.19.1-10.19.11, 1992). Methods for preparation and use of immunoglobulin-based oligomers are well known in the art. One embodiment of the present invention is directed to a dimer comprising two fusion polypeptides created by fusing a polypeptide of the invention to an Fc polypeptide derived from an antibody. A gene fusion encoding the polypeptide/Fc fusion polypeptide is inserted into an appropriate expression vector. Polypeptide/Fc fusion polypeptides are expressed in host cells transformed with the recombinant expression vector, and allowed to assemble much like antibody molecules, whereupon interchain disulfide bonds form between the Fe moieties to yield divalent molecules. One suitable Fe polypeptide, described in PCT application WO 93/10151, is a single chain polypeptide extending from the N-terminal hinge region to the native C-terminus of the Fe region of a human IgG1 antibody. Another useful Fe polypeptide is the Fe mutein described in U.S. Pat. No. 5,457,035 and in Baum et al., (EMBO J. 13:3992-4001, 1994). The amino acid sequence of this mutein is identical to that of the native Fe sequence presented in WO 93/10151, except that amino acid 19 has been changed from Leu to Ala, amino acid 20 has been changed from Leu to Glu, and amino acid 22 has been changed from Gly to Ala. The mutein exhibits reduced affinity for Fe receptors. The above-described fusion polypeptides comprising Fe moieties (and oligomers formed therefrom) offer the advantage of facile purification by affinity chromatography over Polypeptide A or Polypeptide G columns. In other embodiments, the polypeptides of the invention can be substituted for the variable portion of an antibody heavy or light chain. If fusion polypeptides are made with both heavy and light chains of an antibody, it is possible to form an oligomer with as many as four RIPPA and/or RIPPA-Like extracellular regions.

Peptide-linker Based Olijomers. Alternatively, the oligomer is a fusion polypeptide comprising multiple RIPPA and/or RIPPA-Like polypeptides, with or without peptide linkers (spacer peptides). Among the suitable peptide linkers are those described in U.S. Pat. Nos. 4,751,180 and 4,935,233. A DNA sequence encoding a desired peptide linker can be inserted between, and in the same reading frame as, the DNA sequences of the invention, using any suitable conventional technique. For example, a chemically synthesized oligonucleotide encoding the linker can be ligated between the sequences. In particular embodiments, a fusion polypeptide comprises from two to four soluble RIPPA and/or RIPPA-Like polypeptides, separated by peptide linkers. Suitable peptide linkers, their combination with other polypeptides, and their use are well known by those skilled in the art.

Leucine-Zippers. Another method for preparing the oligomers of the invention involves use of a leucine zipper. Leucine zipper domains are peptides that promote oligomerization of the polypeptides in which they are found. Leucine zippers were originally identified in several DNA-binding polypeptides (Landschulz et al., Science 240:1759, 1988), and have since been found in a variety of different polypeptides. Among the known leucine zippers are naturally occurring peptides and derivatives thereof that dimerize or trimerize. The zipper domain (also referred to herein as an oligomerizing, or oligomer-forming, domain) comprises a repetitive heptad repeat, often with four or five leucine residues interspersed with other amino acids. Use of leucine zippers and preparation of oligomers using leucine zippers are well known in the art.

Other fragments and derivatives of the sequences of polypeptides which would be expected to retain polypeptide activity in whole or in part and may thus be useful for, screening or other immunological methodologies can also be made by those skilled in the art given the disclosures herein. Such modifications are believed; to be encompassed by the present invention.

Nucleic Acids Encoding RIPPA and RIPPA-Like Polypeptides

Encompassed within the invention are nucleic acids encoding RIPPA and RIPPA-Like polypeptides. These nucleic acids can be identified in several ways, including isolation of genomic or cDNA-molecules from a suitable source. Nucleotide sequences corresponding to the amino acid sequences described herein, to be used as probes or primers for the isolation of nucleic acids or as query sequences for database searches, can be obtained by “back-translation” from the amino, acid sequences, or by identification of regions of amino acid identity with polypeptides for which the coding DNA sequence has been identified. The well-known polymerase chain reaction (PCR) procedure can be employed to isolate and amplify a DNA sequence encoding a RIPPA or RIPPA-Like polypeptide or a desired combination of RIPPA and/or RIPPA-Like polypeptide fragments. Oligonucleotides that define the desired termini of the combination of DNA fragments are employed as 5′ and 3′ primers. The oligonucleotides can additionally contain recognition sites for restriction endonucleases, to facilitate insertion of the amplified combination of DNA fragments into an expression vector. PCR techniques are described in Saiki et al., Science 239:487 (1988); Recombinant DNA Methodology, Wu et al., eds., Academic Press, Inc., San Diego (1989), pp. 189-196; and PCR Protocols: A Guide to Methods and Applications, Innis et. al., eds., Academic Press, Inc. (1990).

Nucleic acid molecules of the invention include DNA and RNA in both single-stranded and double-stranded form, as well as the corresponding complementary sequences. DNA includes, for example, cDNA, genomic DNA, chemically synthesized DNA, DNA amplified by PCR, and combinations thereof. The nucleic acid molecules of the invention include full-length genes or cDNA molecules as well as a combination of fragments thereof. The nucleic acids of the invention are preferentially derived from human sources, but the invention includes those derived from non-human species, as well.

An “isolated nucleic acid” is a nucleic acid that has been separated from adjacent genetic sequences present in the genome of the organism from which the nucleic acid was isolated, in the case of nucleic acids isolated from naturally-occurring sources. In the case of nucleic acids synthesized enzymatically from a template or chemically, such as PCR products, cDNA molecules, or oligonucleotides for example, it is understood that the nucleic acids resulting from such processes are isolated nucleic acids. An isolated nucleic acid molecule refers to a nucleic acid molecule in the form of a separate fragment or as a component of a larger nucleic acid construct. In one embodiment, the nucleic acids are substantially free from contaminating endogenous material. The nucleic acid molecule has preferably been derived from DNA or RNA isolated at least once in substantially pure form and in a quantity or concentration enabling identification, manipulation, and recovery of its component nucleotide sequences by standard biochemical methods (such as those outlined in Sambrook et al., Molecular Cloning: A Laboratory Manual, 2nd ed., Cold Spring Harbor Laboratory, Cold Spring Harbor, N.Y. (1989)). Such sequences are preferably provided and/or constructed in the form of an open reading frame uninterrupted by internal non-translated sequences, or introns, that are typically present in eukaryotic genes. Sequences of non-translated DNA can be present 5′ or 3′ from an open reading frame, where the same do not interfere with manipulation or expression of the coding region.

The present invention also includes nucleic acids that hybridize under moderately stringent conditions, and highly stringent conditions, to nucleic acids encoding RIPPA or RIPPA-Like polypeptides described herein. The basic parameters affecting the choice of hybridization conditions and guidance for devising suitable conditions are set forth by Sambrook, Fritsch, and Maniatis (1989, Molecular Cloning: A Laboratory Manual, Cold Spring Harbor Laboratory Press, Cold Spring Harbor, N.Y., chapters 9 and 11; and Current Protocols in Molecular Biology, 1995, Ausubel et al., eds., John Wiley & Sons, Inc., sections 2.10 and 6.3-6.4), and can be readily determined by those having ordinary skill in the art based on, for example, the length and/or base composition of the DNA. One way of achieving moderately stringent conditions, involves the use of a prewashing solution containing 5×SSC, 0.5% SDS, 1.0 mM EDTA (pH 8.0), hybridization buffer of about 50% formamide, 6×SSC, and a hybridization temperature of about 55 degrees C. (or other similar hybridization solutions, such as one containing about 50% formamide, with a hybridization temperature of about 42 degrees C.), and washing conditions of about 60 degrees C., in 0.5×SSC, 0.1% SDS. Generally, highly stringent conditions are defined as hybridization conditions as above, but with washing at approximately 68 degrees C., 0.2×SSC, 0.1% SDS. SSPE (1× SSPE is 0.15M NaCl, 10 mM NaH.sub.2 PO.sub.4, and 1.25 mM EDTA, pH 7.4) can be substituted for SSC (1×SSC is 0.15M NaCl and 15 mM sodium citrate) in the hybridization and wash buffers; washes are performed for 15 minutes after hybridization is complete. It should be understood that the wash temperature and wash salt concentration can be adjusted as necessary to achieve a desired degree of stringency by applying the basic principles that govern hybridization reactions and duplex stability, as known to those skilled in the art and described further below (see, e.g., Sambrook et al., 1989). When hybridizing a nucleic acid to a target nucleic acid of unknown sequence, the hybrid length is assumed to be that of the hybridizing nucleic acid. When nucleic acids of known sequence are hybridized, the hybrid length can be determined by aligning the sequences of the nucleic acids and identifying the region or regions of optimal sequence complementarity. The hybridization temperature, for hybrids anticipated to be less than 50 base pairs in length should be 5 to 10 degrees C. less than the melting temperature (Tm) of the hybrid, where Tm is determined according to the following equations. For hybrids less than 18 base pairs in length, Tm (degrees C.)=2(# of A+T bases)+4(# of #G+C bases). For hybrids above 18 base pairs in length, Tm (degrees C.)=81.5+16.6(log₁₀[Na⁺])+0.41(% G+C)−(600/N), where N is the number of bases in the hybrid, and [Na⁺] is the concentration of sodium ions in the hybridization buffer ([Na⁺] for 1×SSC=0.165M). Preferably, each such hybridizing nucleic acid has a length that is at least 15 nucleotides (or at least 18 nucleotides, or at least 20 nucleotides, or at least 25 nucleotides, or at least 30 nucleotides, or at least 40 nucleotides, or at least 50 nucleotides), or at least 25% (at least 50%, or at least 60%, or at least 70%, or at least 80%) of the length of the nucleic acid of the present invention to which it hybridizes, and has at least 60% sequence identity (at least 70%, at least 75%, at least 80%, at least 85%, at least 90%, at least 95%, at least 97.5%, at least 99%, or at least 99.5%) with the nucleic acid of the present invention to which it hybridizes, where sequence identity is determined by comparing the sequences of the hybridizing nucleic acids when aligned so as to maximize overlap and identity while minimizing sequence gaps as described in more detail above.

The present invention also provides genes corresponding to the nucleic acid sequences disclosed herein. “Corresponding genes” or “corresponding genomic nucleic acids” are the regions of the genome that are transcribed to produce the mRNAs from which cDNA nucleic acid sequences are derived and can include contiguous regions of the genome necessary for the regulated expression of such genes. Corresponding genes can therefore include but are not limited to coding sequences, 5′ and 3′ untranslated regions, alternatively spliced exons, introns, promoters, enhancers, and silencer or suppressor elements. Corresponding genomic nucleic acids can include 10000 basepairs (or 5000 basepairs, or 2500 basepairs, or 1000 basepairs) of genomic nucleic acid sequence upstream of the first nucleotide of the genomic sequence corresponding to the initiation codon of the RIPPA or RIPPA-Like coding sequence, and 10000 basepairs (or 5000 basepairs, or 2500 basepairs, or 1000 basepairs) of genomic nucleic acid sequence downstream of the last nucleotide of the genomic sequence corresponding to the termination codon of the RIPPA or RIPPA-Like coding sequence. The corresponding genes or genomic nucleic acids can be isolated in accordance with known methods using the sequence information disclosed herein. Such methods include the preparation of probes or primers from the disclosed sequence information for identification and/or amplification of genes in appropriate genomic libraries or other sources of genomic materials. An “isolated gene” or “an isolated genomic nucleic acid” is a genomic nucleic acid that has been separated from the adjacent genomic sequences present in the genome of the organism from which the genomic nucleic acid was isolated.

Methods for Making and Purifying RIPPA and RIPPA-Like Polypeptides

Methods for making RIPPA and RIPPA-Like polypeptides are described below. Expression, isolation, and purification of the polypeptides and fragments of the invention can be accomplished by any suitable technique, including but not limited to the following methods. In one embodiment, host cells for producing recombinant RIPPA polypeptides are COS cells.

The isolated nucleic acid of the invention can be operably linked to an expression control sequence such as the pDC409 vector (Giri et al., 1990, EMBO J., 13: 2821) or the pDC412 vector (Wiley et al., 1995, Immunity 3: 673). The pDC400 series vectors are useful for transient expression in mammalian cells such as CV-1 or 293 cells. Alternatively, the isolated nucleic acid of the invention can be linked to expression vectors such as the pDC300 series vectors, which are useful for stable mammalian expression in cells such as CHO cells or their derivatives. Other expression control sequences and cloning technologies can also be used to produce the polypeptide recombinantly, such as the pMT2 or pED expression vectors (Kaufman et al., 1991, Nucleic Acids Res. 19: 44854490; and Pouwels et al., 1985, Cloning Vectors: A Laboratory Manual, Elsevier, New York) and the GATEWAY Vectors (Life Technologies; Rockville, Md.). Many suitable expression control sequences and general methods of expressing recombinant polypeptides are known in the art (R. Kaufman, Methods in Enzymology 185, 537-566 (1990)). As used herein “operably linked” means that the nucleic acid of the invention and an expression control sequence are situated within a construct, vector, or cell in such a way that the polypeptide encoded by the nucleic acid is expressed when appropriate molecules (such as polymerases) are present. As one embodiment of the invention, at least one expression control sequence is operably linked to the nucleic acid of the invention in a recombinant host cell or progeny thereof, the nucleic acid and/or expression control sequence having been introduced into the host cell by transformation or transfection, or by any other suitable method. As another embodiment of the invention, at least one expression control sequence is integrated into the genome of a recombinant host cell such that it is operably linked to a nucleic acid sequence encoding a polypeptide of the invention. In a further embodiment of the invention, at least one expression control sequence is operably linked to a nucleic acid of the invention through the action of a trans-acting factor such as a transcription factor, either in vitro or in a recombinant host cell. A sequence encoding an appropriate signal peptide (native or heterologous) can also be incorporated into expression vectors. The choice of signal peptide or leader can depend on factors such as the type of host cells in which the recombinant polypeptide is to be produced. Examples of heterologous signal peptides that are functional in mammalian host cells are described in U.S. Pat. No. 4,965,195; Cosman et al., Nature 312:768 (1984); EP 367,566; U.S. Pat. No. 4,968,607; and EP 460,846. A DNA sequence for a signal peptide (secretory leader) can be fused in frame to the nucleic acid sequence of the invention so that the DNA is initially transcribed, and the mRNA translated, into a fusion polypeptide comprising the signal peptide. A signal peptide that is functional in the intended host cells is one that promotes insertion of the polypeptide into cell membranes, and most preferably, promotes extracellular secretion of the polypeptide from that host cell. The signal peptide is preferably cleaved from the polypeptide upon membrane insertion or secretion of polypeptide from the cell. The skilled artisan will also recognize that the position(s) at which the signal peptide is cleaved can vary according to such factors as the type of host cells employed in expressing a recombinant polypeptide. A polypeptide preparation can include a mixture of polypeptide molecules having different N-terminal amino acids, resulting from cleavage of the signal peptide at more than one site.

Established methods for introducing DNA into mammalian cells have been described (Kaufman, R. J., Large Scale Mammalian Cell Culture, 1990, pp. 15-69). Additional protocols using commercially available reagents, such as Lipofectamine lipid reagent (Gibco/BRL) can be used to transfect cells (Felgner et al., Proc. Natl. Acad. Sci. USA 84:7413-7417, 1987). Electroporation can also be used to transfect mammalian cells using conventional procedures, such as those in Sambrook et al. (Molecular Cloning: A Laboratory Manual, 2 ed. Vol. 1-3, Cold Spring Harbor Laboratory Press, 1989). Selection of stable transformants can be performed using methods known in the art, such as, for example, resistance to cytotoxic drugs such as dihydrofolate reductase (Kaufman et al., Meth. in Enzymology 185:487-511, 1990). Other examples of selectable markers that can be incorporated into an expression vector include cDNAs conferring resistance to antibiotics such as G418 and hygromycin B. Cells harboring the vector can be selected on the basis of resistance to these compounds. Alternatively, RIPPA and RIPPA-Like gene products can be obtained via homologous recombination, or “gene targeting,” techniques. Such techniques employ the introduction of exogenous transcription control elements (such as the CMV promoter or the like) in a particular predetermined site on the genome, to induce expression of the endogenous nucleic acid sequence of interest (see, for example, U.S. Pat. No. 5,272,071). The location of integration into a host chromosome or genome can be easily determined by one of skill in the art, given the known location and sequence of the gene. In a preferred embodiment, the present invention also contemplates the introduction of exogenous transcriptional control elements in conjunction with an amplifiable gene, to produce increased amounts of the gene product, again, without the need for isolation of the gene sequence itself from the host cell.

A number of types of cells can act as suitable host cells for expression of the polypeptide. Mammalian host cells include, for example, the COS-7 line of monkey kidney cells (ATCC CRL 1651), L cells, C127 cells, 3T3 cells (ATCC CCL 163), Chinese hamster ovary (CHO) cells or their derivatives such as Veggie CHO and related cell lines which grow in serum-free media (Rasmussen et al., 1998, Cytotechnology 28: 31), HeLa cells, BHK (ATCC CRL 10) cell lines, the CV1/EBNA cell line (ATCC CCL 70), human embryonic kidney cells such as 293, 293 EBNA, or MSR 293, human epidermal A431 cells, human Colo205 cells, other transformed primate cell lines, normal diploid cells, cell strains derived from in vitro culture of primary tissue, primary explants, HL-60, U937, HaK or Jurkat cells. Optionally, mammalian cell lines such as HepG2/3B, KB, NIH 3T3, or S49, for example, can be used for expression of the polypeptide when it is desirable to use the polypeptide in various signal transduction or reporter assays. Alternatively, it is possible to produce the polypeptide in lower eukaryotes such as yeast or in prokaryotes such as bacteria. Suitable yeasts include Saccharomyces cerevisiae, Schizosaccharomyces pombe, Kluyveromyces strains, Candida, or any yeast strain capable of expressing heterologous polypeptides. Suitable bacterial strains include Escherichia coli, Bacillus subtilis, Salmonella typhimurium, or any bacterial strain capable of expressing heterologous polypeptides. If the polypeptide is made in yeast or bacteria, it may be desirable to modify the polypeptide produced therein, for example by phosphorylation or glycosylation of the appropriate sites, in order to obtain the functional polypeptide. Such covalent attachments can be accomplished using known chemical or enzymatic methods. The polypeptide can also be produced by operably linking the isolated nucleic acid of the invention to suitable control sequences in one or more insect expression vectors, and employing an insect expression system (Summers and Smith, Texas Agricultural Experiment Station Bulletin No. 1555 (1987), and Luckow and Summers, Bio/Technology 6:47 (1988)). Cell-free translation systems could also be employed to produce polypeptides using RNAs derived from nucleic acid constructs disclosed herein. A host cell that comprises an isolated nucleic acid of the invention, preferably operably linked to at least one expression control sequence, is a “recombinant host cell”. The polypeptide of the invention can also be expressed as a product of transgenic animals, e.g., as a component of the milk of transgenic cows, goats, pigs, or sheep which are characterized by somatic or germ cells containing a nucleotide sequence encoding the polypeptide.

The polypeptide of the invention can be prepared by culturing transformed host cells under culture conditions suitable to express the recombinant polypeptide. The resulting expressed polypeptide can then be purified from such culture (i.e., from culture medium or cell extracts) using known purification processes, such as selective precipitation with various salts, gel filtration, and ion exchange chromatography. The purification of the polypeptide can also include an affinity column containing agents which will bind to the polypeptide; one or more column steps over such affinity resins: as concanavalin A-agarose, heparin-toyopearl® or Cibacrom blue 3GA Sepharose®; one or more steps involving hydrophobic interaction chromatography using such resins as phenyl ether, butyl ether, or propyl ether; or immunoaffinity chromatography using an, antibody that specifically binds one or more RIPPA and/or RIPPA-Like epitopes. Alternatively, the polypeptide of the invention can also be expressed in a form which will facilitate purification: For example, it can be expressed as a fusion polypeptide, that is, it may be fused with maltose binding polypeptide (MBP), glutathione-S-transferase (GST), thioredoxin (TRX), a polyHis peptide, and/or fragments thereof. The polypeptide can also be tagged with an epitope and subsequently purified by using a specific antibody directed to such epitope. One such epitope (FLAG®) is commercially available from Kodak (New Haven, Conn.). Finally, one or more reverse-phase high performance liquid chromatography (RP-HPLC) steps employing hydrophobic RP-HPLC media can be employed to further purify the polypeptide. Some or all of the foregoing purification steps, in various combinations, can also be employed to provide a substantially homogeneous isolated recombinant polypeptide. The polypeptide thus purified is substantially free of other mammalian polypeptides and is defined in accordance with the present invention as an “isolated polypeptide”; such isolated polypeptides of the invention include isolated antibodies that bind to RIPPA and/or RIPPA-Like polypeptides, fragments, variants, binding partners etc. The desired degree of purity depends on the intended use of the polypeptide. A relatively high degree of purity is desired when the polypeptide is to be administered in vivo, for example. In such a case, the polypeptides are purified such that no polypeptide bands corresponding to other polypeptides are detectable upon analysis by SDS-polyacrylamide gel electrophoresis (SDS-PAGE). It will be recognized by one skilled in the art that multiple bands corresponding to the polypeptide can be visualized by SDS-PAGE, due to differential glycosylation, differential post-translational processing, and the like. The polypeptide of the invention can be purified to substantial homogeneity, as indicated by a single polypeptide band upon analysis by SDS-PAGE.

The polypeptide can also be produced by known conventional chemical synthesis. Methods for constructing the polypeptides of the present invention by synthetic means are known to those skilled in the art. The synthetically-constructed polypeptide sequences, by virtue of sharing primary, secondary or tertiary structural and/or conformational characteristics with RIPPA and/or RIPPA-Like polypeptides can possess biological properties in common therewith, including RIPPA polypeptide activity. Thus, they can be employed as biologically active or immunological substitutes for natural, purified polypeptides in screening of therapeutic compounds and in immunological processes for the development of antibodies.

Antagonists and Agonists of RIPPA and RIPPA-Like Polypeptides

Any method which neutralizes RIPPA and/or RIPPA-Like polypeptides or modulates the biological effects of RIPPA and/or RIPPA-Like polypeptides or inhibits expression of the RIPPA and/or RIPPA-Like genes (either, transcription or translation) can be used to reduce the biological activities of RIPPA and RIPPA-Like polypeptides. In particular embodiments, antagonists inhibit the binding of at least one RIPPA or RIPPA-Like polypeptide to binding partners, thereby inhibiting biological activities induced by the binding of those RIPPA and/or RIPPA-Like polypeptides to the binding partners. In certain other embodiments of the invention, antagonists can be designed to reduce the level of endogenous RIPPA or RIPPA-Like gene expression, e.g., using well-known antisense or ribozyme approaches to inhibit or prevent translation of RIPPA or RIPPA-Like mRNA transcripts; triple helix approaches to inhibit transcription of RIPPA or RIPPA-Like family genes; targeted homologous recombination to inactivate or “knock out” the RIPPA or RIPPA-Like genes or their endogenous promoters or enhancer elements; or using double-stranded RNA to target specific mRNAs for degradation and thereby silencing their expression, such as RNA interference (RNAi) and other RNA silencing phenomena found in plants, animals and fungi. Such antisense, ribozyme, triple helix antagonists and RNAi sequences can be designed to reduce or inhibit either unimpaired, or if appropriate, mutant RIPPA or RIPPA-Like gene activity. Techniques for the production and use of such molecules are well known to those of skill in the art.

RIPPA and RIPPA-Like polypeptides are linked to osteoclastogenesis and/or osteoclastic bone resorption processes and therefore RIPPA and RIPPA-Like polypeptides are implicated in diseases or conditions characterized by excessive bone resorption, generally referred to as osteopenias. Further embodiments are drawn to treating conditions and diseases that share cation exchange disregulation as a common feature in their etiology. More specifically, the biological activities of RIPPA and RIPPA-Like polypeptides are likely involved in the following medical conditions: osteoporosis, osteomyelitis, hypercalcemia, osteopenia brought on by surgery or steroid administration, prosthetic loosening, Paget's disease, osteonecrosis, bone loss due to rheumatoid arthritis, periodontal bone loss, and and cancers that may metastasize to bone and induce bone breakdown (i.e., multiple myeloma, breast cancer, some melanomas; see also Mundy, C. Cancer Suppl. 80:1546; 1997).

Blocking or inhibiting the interactions between members of the RIPPA and RIPPA-Like polypeptide family and their substrates, ligands, receptors, binding partners, and or other interacting polypeptides is an aspect of the invention and provides methods for treating or ameliorating these diseases and conditions through the use of inhibitors of RIPPA and/or and RIPPA-Like polypeptide activity. Examples of such inhibitors or antagonists are described in more detail below. For certain conditions involving too little RIPPA or and RIPPA-Like polypeptide activity, methods of treating or ameliorating these conditions comprise increasing the amount or activity of RIPPA or and RIPPA-Like polypeptides by providing, isolated RIPPA or and RIPPA-Like polypeptides or active fragments or fusion polypeptides thereof, or by providing compounds (agonists) that activate endogenous or exogenous RIPPA and/or and RIPPA-Like polypeptides. Preferred methods of administering RIPPA and and RIPPA-Like polypeptides to organisms in need of treatment, such as mammals or most preferably humans, include in vivo or ex vivo treament of cells with viral particles or liposomes containing nucleic acids encoding RIPPA and/or and RIPPA-Like polypeptides to be expressed in target cells of the organism in need of treatment.

Antisense RNA and DNA molecules act to directly block the translation of mRNA by hybridizing to targeted mRNA and preventing polypeptide translation. Antisense approaches involve the design of oligonucleotides (either DNA or RNA) that are complementary to a RIPPA or RIPPA-Like mRNA. The antisense oligonucleotides will bind to the complementary target gene mRNA transcripts and prevent translation. Absolute complementarity, although preferred, is not required. A sequence “complementary” to a portion of a nucleic acid, as referred to herein, means a sequence having sufficient complementarity to be able to hybridize with the nucleic acid, forming a stable duplex (or triplex, as appropriate). In the case of double-stranded antisense nucleic acids, a single strand of the duplex DNA can thus be tested, or triplex formation can be assayed. The ability to hybridize will depend on both the degree of complementarity and the length of the antisense nucleic acid. Preferred oligonucleotides are complementary to the 5′ end of the message, e.g., the 5′ untranslated sequence up to and including the AUG initiation codon. However, oligonucleotides complementary to the 5′- or 3′-non-translated, non-coding regions of the RIPPA or RIPPA-Like gene transcript, or to the coding regions, could be used in an antisense approach to inhibit translation of endogenous RIPPA or RIPPA-Like mRNA. Antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides. The oligonucleotides can be DNA or RNA or chimeric mixtures or derivatives or modified versions thereof, single-stranded or double-stranded. Chimeric oligonucleotides, oligonucleosides, or mixed oligonucleotides/oligonucleosides of the invention can be of several different types. These include a first type wherein the “gap” segment of nucleotides is positioned between 5′ and 3′ “wing” segments of linked nucleosides and a second “open end” type wherein the “gap” segment is located at either the 3′ or the 5′ terminus of the oligomeric compound (see, e.g., U.S. Pat. No. 5,985,664). Oligonucleotides of the first type are also known in the art as “gapmers” or gapped oligonucleotides. Oligonucleotides of the second type are also known in the art as “hemimers” or “wingmers”. The oligonucleotide can be modified at the base moiety, sugar moiety, or phosphate backbone, for example, to improve stability of the molecule, hybridization, etc. The oligonucleotide can include other appended groups such as peptides (e.g., for targeting host cell receptors in vivo), or agents facilitating transport across the cell membrane (see, e.g., Letsinger et al., 1989, Proc Natl Acad Sci U.S.A. 86: 6553-6556; Lemaitre et al., 1987, Proc Natl Acad Sci 84: 648-652; PCT Publication No. WO88/09810), or hybridization-triggered cleavage agents or intercalating agents. (See, e.g., Zon, 1988, Pharm. Res. 5: 539-549). The antisense molecules should be delivered to cells which express the RIPPA and/or RIPPA-Like transcript in vivo. A number of methods have been developed for delivering antisense DNA or RNA to cells; e.g., antisense molecules can be injected directly into the tissue or cell derivation site, or modified antisense molecules, designed to target the desired cells (e.g., antisense linked to peptides or antibodies that specifically bind receptors or antigens expressed on the target cell surface) can be administered systemically. However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous RIPPA or RIPPA-Like gene transcripts and thereby prevent translation of the RIPPA or RIPPA-Like mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells.

Ribozyme molecules designed to catalytically cleave RIPPA or RIPPA-Like mRNA transcripts can also be used to prevent translation of RIPPA or RIPPA-Like mRNA and expression of RIPPA or RIPPA-Like polypeptides. (See, e.g., PCT International Publication W090/11364 and U.S. Pat. No. 5,824,519). The ribozymes that can be used in the present invention include hammerhead ribozymes (Haseloff and Gerlach, 1988, Nature, 334:585-591), RNA endoribonucleases (hereinafter “Cech-type ribozymes”) such as the one which occurs naturally in Tetrahymena Thermophila (known as the IVS, or L-19 IVS RNA) and which has been extensively described by Thomas Cech and collaborators (International Patent Application No. WO 88/04300; Been and Cech, 1986, Cell, 47:207-216). As in the antisense approach, the ribozymes can be composed of modified oligonucleotides (e.g. for improved stability, targeting, etc.) and should be delivered to cells which express the RIPPA or RIPPA-Like polypeptide in vivo. A preferred method of delivery involves using a DNA construct “encoding” the ribozyme under the control of a strong constitutive pol III or pol II promoter, so that transfected cells will produce sufficient quantities of the ribozyme to destroy endogenous RIPPA or RIPPA-Like messages and inhibit translation: Because ribozymes unlike antisense molecules, are catalytic, a lower intracellular concentration is required for efficiency.

Alternatively, endogenous RIPPA or RIPPA-Like gene expression can be reduced by targeting deoxyribonucleotide sequences complementary to the regulatory region of the target gene (i.e., the target gene promoter and/or enhancers) to form triple helical structures that prevent transcription of the target RIPPA or RIPPA-Like gene. (See generally, Helene, 1991, Anticancer Drug Des., 6(6), 569-584; Helene, et al., 1992, Ann. N.Y. Acad. Sci., 660, 27-36; and Maher, 1992, Bioassays 14(12), 807-815).

Anti-sense RNA and DNA, ribozyme, and triple helix molecules of the invention can be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Oligonucleotides can be synthesized by standard methods known in the art, e.g. by use of an automated DNA synthesizer (such as are commercially available from Biosearch, Applied Biosystems, etc.). As examples, phosphorothioate oligonucleotides can be synthesized by the method of Stein et al., 1988, Nucl. Acids Res. 16:3209. Methylphosphonate oligonucleotides can be prepared by use of controlled pore glass polymer supports (Sarin et al., 1988, Proc. Natl. Acad. Sci. U.S.A. 85:7448-7451). Alternatively, RNA molecules can be generated by in vitro and in vivo transcription of DNA sequences encoding the antisense RNA molecule. Such DNA sequences can be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Endogenous target gene expression can also be reduced by inactivating or “knocking out” the target gene or its promoter using targeted homologous recombination (e.g., see Smithies, et al., 1985, Nature 317, 230-234; Thomas and Capecchi, 1987, Cell 51, 503-512; Thompson, et al., 1989, Cell 5, 313-321). For example, a mutant, non-functional target gene (or a completely unrelated DNA sequence) flanked by DNA homologous to the endogenous target gene (either the coding regions or regulatory regions of the target gene) can be used, with or without a selectable marker and/or a negative selectable marker, to transfect cells that express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene (e.g., see Thomas and Capecchi, 1987 and Thompson, 1989, supra), or in model organisms such as Caenorhabditis elegans where the “RNA interference” (“RNAi”) technique (Grishok, Tabara, and Mello, 2000, Genetic requirements for inheritance of RNAi in C. elegans, Science 287 (5462): 2494-2497), or the introduction of transgenes (Dernburg et al., 2000, Transgene-mediated cosuppression in the C. elegans germ line, Genes Dev. 14 (13): 1578-1583) are used to inhibit the expression of specific target genes. However this approach can be adapted for use in humans provided the recombinant DNA constructs are directly administered or targeted to the required site in vivo using appropriate vectors such as viral vectors.

Organisms that have enhanced, reduced, or modified expression of the gene(s) corresponding to the nucleic acid sequences disclosed herein are provided. The desired change in gene expression can be achieved through the use of antisense nucleic acids or ribozymes that bind and/or cleave the mRNA transcribed from the gene (Albert and Morris, 1994, Trends Pharmacol. Sci. 15(7): 250-254; Lavarosky et al., 1997, Biochem. Mol. Med. 62(1): 11-22; and Hampel, 1998, Prog. Nucleic Acid Res. Mol. Biol. 58: 1-39). Transgenic animals that have multiple copies of the gene(s) corresponding to the nucleic acid sequences disclosed herein, preferably produced by transformation of cells with genetic constructs that are stably maintained within the transformed cells and their progeny, are provided. Transgenic animals that have modified genetic control regions that increase or reduce gene expression levels, or that change temporal or spatial patterns of gene expression, are also provided (see European Patent No. 0 649 464 B1). In addition, organisms are provided in which the gene(s) corresponding to the nucleic acid sequences disclosed herein have been partially or completely inactivated, through insertion of extraneous sequences into the corresponding gene(s) or through deletion of all or part of the corresponding gene(s). Partial or complete gene inactivation can be accomplished through insertion, preferably followed by imprecise excision, of transposable elements (Plasterk, 1992, Bioessays 14(9): 629-633; Zwaal et al., 1993, Proc. Natl. Acad. Sci. USA 90(16): 7431-7435; Clark et al., 1994, Proc. Natl. Acad. Sci. USA 91(2): 719-722), or through homologous recombination, preferably detected by positive/negative genetic selection strategies (Mansour et al., 1988, Nature 336: 348-352; U.S. Pat. Nos. 5,464,764; 5,487,992; 5,627,059; 5,631,153; 5,614,396; 5,616,491; and 5,679,523). These organisms with altered gene expression are preferably eukaryotes and are mammals. Such organisms are useful for the development of non-human models for the study of disorders involving the corresponding gene(s), and for the development of assay systems for the identification of molecules that interact with the polypeptide product(s) of the corresponding gene(s).

Also encompassed within the invention are RIPPA or RIPPA-Like polypeptide variants with partner binding sites that have been altered in conformation so that (1) the RIPPA or RIPPA-Like variant will still bind to its partner(s), but a specified small molecule will fit into the altered binding site and block that interaction, or (2) the RIPPA or RIPPA-Like variant will no longer bind to its partner(s) unless a specified small molecule is present (see for example Bishop et al., 2000, Nature 407: 395-401). Nucleic acids encoding such altered RIPPA and/or RIPPA-Like polypeptides can be introduced into organisms according to methods described herein, and can replace the endogenous nucleic acid sequences encoding the corresponding RIPPA and/or RIPPA-Like polypeptide. Such methods allow for the interaction of a particular RIPPA and/or RIPPA-Like polypeptide with its binding partners to be regulated by administration of a small molecule compound to an organism, either systemically or in a localized manner.

The RIPPA and RIPPA-Like polypeptides themselves can also be employed in inhibiting a biological activity of RIPPA and/or RIPPA-Like in in vitro or in vivo procedures. Encompassed within the invention are cytoplasmic C-terminal domains of RIPPA or RIPPA-Like polypeptides that act as “dominant negative” inhibitors of native RIPPA and/or RIPPA-Like polypeptide function when expressed as fragments or as components of fusion polypeptides. For example, a purified polypeptide domain of the present invention can be used to inhibit binding of RIPPA and/or RIPPA-Like polypeptides to endogenous binding partners. Such use effectively would block RIPPA and/or RIPPA-Like polypeptide interactions and inhibit RIPPA polypeptide activities. Furthermore, antibodies which bind to RIPPA and/or RIPPA-Like polypeptides often inhibit RIPPA and/or RIPPA-Like polypeptide activity and act as antagonists. For example, antibodies that specifically recognize one or more epitopes of RIPPA and/or RIPPA-Like polypeptides, or epitopes of conserved variants of RIPPA and/or RIPPA-Like polypeptides, or peptide fragments of the RIPPA and/or RIPPA-Like polypeptide can be used in the invention to inhibit RIPPA and/or RIPPA-Like polypeptide activity. Such antibodies include but are not limited to polyclonal antibodies, monoclonal antibodies (nAbs), humanized or chimeric antibodies, single chain antibodies, Fab fragments, F(ab′)2 fragments, fragments produced by a Fab expression library, anti-idiotypic (anti-Id) antibodies, and epitope-binding fragments of any of the above. Alternatively, purified and modified RIPPA and/or RIPPA-Like polypeptides of the present invention can be administered to modulate interactions between RIPPA and/or RIPPA-Like polypeptides and RIPPA and/or RIPPA-Like binding partners that are not membrane-bound. Such an approach will allow an alternative method for the modification of RIPPA-influenced bioactivity.

In alternative aspects, the invention further encompasses the use of agonists of RIPPA polypeptide activity to treat or ameliorate the symptoms of a disease for which increased RIPPA and/or RIPPA-Like polypeptide activity is beneficial. The use of agonists to modulate the biological effects of RIPPA and RIPPA-Like polypeptides may be used to treat diseases or conditions characterized by a decrease in the rate of bone resportion by osteoclasts, generally referred to as osteopetrosis, which is often characterized by excessive bone density. In a one aspect, the invention entails administering compositions comprising a RIPPA and/or RIPPA. Like nucleic acid or a RIPPA and/or RIPPA-Like polypeptide to cells in vitro, to cells ex vivo, to cells in vivo, and/or to a multicellular organism such as a vertebrate or mammal. Preferred therapeutic forms of RIPPA and/or RIPPA-Like polypeptides are soluble forms, as described above. In still another aspect of the invention, the compositions comprise administering a RIPPA- or RIPPA-Like-encoding nucleic acid for expression of a RIPPA or RIPPA-Like polypeptide in a host organism for treatment of disease. Particularly preferred in this regard is expression in a human patient for treatment of a dysfunction associated with aberrant (e.g., decreased) endogenous activity of a RIPPA and/or RIPPA-Like family polypeptide. Furthermore, the invention encompasses the administration to cells and/or organisms of compounds found to increase the endogenous activity of RIPPA and/or RIPPA-Like polypeptides. One example of compounds that increase RIPPA and/or RIPPA-Like polypeptide activity are agonistic antibodies, preferably monoclonal antibodies, that bind to RIPPA and/or RIPPA-Like polypeptides or binding partners, which may increase RIPPA and/or RIPPA-Like polypeptide activity by causing constitutive intracellular signaling (or “ligand mimicking”), or by preventing the binding of a native inhibitor of RIPPA polypeptide activity.

Assays for determining the effects of RIPPA and/or RIPPA-Like agonists and antagonists include, but are not limited to, culturing monocyte/macrophage cells in vitro and inducing said cells to mature/differentiate to osteoclasts. These cells are cultured on bone or dentin whereby the osteoclasts excavate resorptive lacunae in the bone or dentin substrate. Alternatively, the cells may be cultured on calcium phophate matrices, such as those described in Langstaff, S., et al, Biomaterials, 2001, Jan;22(2):135-50 or commercially available from sources such as the Biosciences Division of Bectin Dickinson, Frannklin Lakes, N.J. The number and size of resorption lacunae formed in the bone, dentin or calcium phophate substrate are a quantitative measure of osteoclast activity (see, Fuller, K., et al. J. Bone Miner. Res. 1994, 9:17). RIPPA and/or RIPPA-Like agonists or antagonists may be added to the assay at any time point to determine if the RIPPA and/or RIPPA-Like agonists or antagonists cause an increase or decrease in the relative number and/or size of resorption lacunae. A quantitative measurement of the number and/or size of resorption lacunae formed in the bone, dentin or calcium phosphate substrate is performed using standard techniques to ascertain differences between cultures receiving a RIPPA and/or RIPPA-Like agonists or antagonists and those that did not. Cultures receiving RIPPA and/or RIPPA-Like agonists or antagonists that have an increased or decreased relative number and/or size of resorption lacunae indicate a potential candidate for therapeutic use in modulating RIPPA and/or RIPPA-Like activities. Additional in vitro screening assays for osteoclast activity may be used to screen for RIPPA and/or RIPPA-Like agonists and/or antagonitsts, such as those using biotinylated bone, dentin or calcium phosphate substrates, as described in Nesbitt, S. A., et al. 1997, Science 276:266.

In vitro screening assay may be used to determine the effects of RIPPA and/or RIPPA-Like agonists and/or antagonitsts on osteoclastogenesis. Biological models of osteoclast differentiation have been developed that facilitate the detailed study the factors involved in the regulation of this process. One embodiment comprises cultures of mouse bone marrow or cocultures of haematopoietic cells with bone-derived stromal cells, which give rise to large numbers of bone-resorbing oseoclasts. One of skill in the art would be familiar with such assays, such as those described in Rodman, G. D., Experimental Hematology 1999, 27:1229-1241; Suda, T., et al. Endocr Rev 1992, 13:66-80; Takahashi, N., et al. Endocrinology 1998, 123:2600-2602; Quinn, J. M., et al. Endocrinology 1994, 134:2416-2423; Kurihara, N., et al. J Bone Miner Res 1991, 6:257-261; Matayoshi, A., et al. PNAS 1996, 93:10785-10790; and, Roux, S., et al. J Cell Physiol 1996, 168:489498. In alternative embodiments, in vitro screening assays include culturing established monocyte/macrophage cell lines or primary monocyte cultures, and inducing said cells to mature/differentiate to osteoclasts, as described in detail in Examples 4 and 5.

Additional readouts may be used to determine the effects of RIPPA and/or RIPPA-Like agonists and/or antagonists in the in vitro assays described above, such as monitoring surrogate markers of osteoclastogenesis or osteoclastic bone resorption. For example, the TRAP (Tartrate-Resistant Acid Phosphatase) assay may be used to monitor tartrate-resistant acid phosphatase (also referred to as type 5 acid phosphatase), which is a marker enzyme of bone-resorbing osteoclasts. Cells from the assays described above may also be stained for TRAP using standard histological or immunohistochemical techniques. In alternative assays, culture or cellular levels of osteocalcin may be monitored as an indicator of recently formed bone. Osteoclacin is a protein specifically produced by osteoblasts and is an integral component of bone formation; commercial kits are available, but any suitable method or assay for measuring culture or cellular levels of osteocalcin may be used.

In alternative embodiments, reporter assays may be used to measure the effects of RIPPA and/or RIPPA-Like agonists and/or antagonists on osteoclastogenesis or osteoclastic bone resorption in in vitro assays. Reporter assays are well known in the art and are readily amenable to this analysis. Examples include, but are not limited to, analytical chemiluminescence and bioluminescence, expression of cell surface molecules, expression and release of soluble biomolecules, and the like. In one embodiment a construct is created comprising the mmP9 promoter operably linked to polynucleotide sequences encoding a cytokine, such as IL-2. This contruct is transfected into osteoclast precursors, such as the RAW 264.7 macrophage cell line. Upon stimulation by one or more factors that cause the cells to differentiate into osteoclasts, such as exposure to RANK-L, the mmP9 promoter is activated and IL-2 is released as the reporter and is indicative of osteoclastogenesis and/or osteclastogenic activity.

RIPPA and/or RIPPA-Like agonists and/or antagonists may be tested in vivo. Bone collagens are extensively degraded by the action of collagenolytic enzymes during resorption and the resultant release of type I collagen fragments into the extracellular space may be detected in the plasma and urine, thereby providing a clinical measurement of bone resorption. RIPPA and/or RIPPA-Like agonists and/or antagonists are administered to a subject and relative amounts of type I collagen in the plasma and/or urine are measured. Subjects receiving RIPPA and/or RIPPA-Like agonists or antagonists that have an increase or decrease in the relative amount of type I collagen in the plasma and/or urine indicate a potential candidate for therapeutic use in modulating RIPPA and/or RIPPA-Like activities. Additional assays for monitoring in vivo testing of RIPPA and/or RIPPA-Like agonists and/or antagonists include the TRAP assay (commercially available assays include BoneTRAP® assay (Suomen Bioanalytiikka Oy, Turku, Finland). In general, TRAP, tartrate-resistant acid phosphatase, is secreted into the circulation by osteoclasts and it has been shown that circulating serum levels of TRAP is a useful marker of bone resorption activity. In alternative assays, serum levels of osteocalcin may be monitored as an indicator of recently formed bone. Osteoclacin is a protein specifically produced by osteoblasts and is an integral component of bone formation. Commercial kits are available, such as the IMMULITE® Osteocalcin Assay (Diagnotic Products Corp., Los Angeles, Calif.), but any suitable method or assay for measuring serum levels of osteocalcin may be used.

In alternative in vivo assays, Dual-energy X-ray Absorptiometry (DEXA) for bone mineral density determination is well-established in the art and may be used to assess the qualitative and quantitative differences between subjects receiving RIPPA and/or RIPPA-Like agonists or antagonists and subjects that did not. Additional embodiments of monitoring bone density and therefore the efficacy of RIPPA and/or RIPPA-Like agonists or antagonists include for example, single-photon absorptiometry, dual-photon absorptiometry, quantitative computed tomography and radiographic absorptiometry. Also, traditional pathological, histological and/or immunohistochemical staining of tissues may be used to assess pathological differences between study groups.

Antibodies to RIPPA and/or RIPPA-Like Polypeptides

Antibodies that are immunoreactive with the polypeptides of the invention are provided herein. Alternative embodiments include antibodies that are agonists or antagonists to RIPPA and/or RIPPA-Like polypeptides, fragments, variants, fusion polypeptides, etc. and modulate the biological activities and functions of RIPPA and/or RIPPA-Like polypeptides, fragments, variants, fusion polypeptides, etc. Such antibodies specifically bind to the polypeptides via the antigen-binding sites of the antibody (as opposed to non-specific binding). In the present invention, specifically binding antibodies are those that will specifically recognize and bind with RIPPA and/or RIPPA-Like polypeptides, homologues, and variants, but not with other molecules. In one preferred embodiment, the antibodies are specific for the polypeptides of the present invention and do not cross-react with other polypeptides. In this manner, the RIPPA and/or RIPPA-Like polypeptides, fragments, variants, fusion polypeptides, etc., as set forth above can be employed as “immunogens” in producing antibodies immunoreactive therewith.

More specifically, the polypeptides, fragment, variants, fusion polypeptides, etc. contain antigenic determinants or epitopes that elicit the formation of antibodies. These antigenic determinants or epitopes can be either linear or conformational (discontinuous). Linear epitopes are composed of a single section of amino acids of the polypeptide, while conformational or discontinuous epitopes are composed of amino acids sections from different regions of the polypeptide chain that are brought into close proximity upon polypeptide folding (Janeway and Travers, Immuno Biology 3:9 (Garland Publishing Inc., 2nd ed. 1996)). Because folded polypeptides have complex surfaces, the number of epitopes available is quite numerous; however, due to the conformation of the polypeptide and steric hindrances, the number of antibodies that actually bind to the epitopes is less than the number of available epitopes (Janeway and Travers, Immuno Biology 2:14 (Garland Publishing Inc., 2nd ed. 1996)). Epitopes can be identified by any of the methods known in the art. Thus, one aspect of the present invention relates to the antigenic epitopes of the polypeptides of the invention. Such epitopes are useful for raising antibodies, in particular monoclonal antibodies, as described in more detail below. Additionally, epitopes from the polypeptides of the invention can be used as research reagents, in assays, and to purify specific binding antibodies from substances such as polyclonal sera or supernatants from cultured hybridomas. Such epitopes or variants thereof can be produced using techniques well known in the art such as solid-phase synthesis, chemical or enzymatic cleavage of a polypeptide, or using recombinant DNA technology.

As to the antibodies that can be elicited by the epitopes of the polypeptides of the invention, whether the epitopes have been isolated or remain part of the polypeptides, both polyclonal and monoclonal antibodies can be prepared by conventional techniques. See, for example, Monoclonal Antibodies, Hybridomas: A New Dimension in Biological Analyses, Kennet et al. (eds.), Plenum Press, New York (1980); and Antibodies: A Laboratory Manual, Harlow and Land (eds.), Cold Spring Harbor Laboratory Press, Cold Spring Harbor, N.Y., (1988); Kohler and Milstein, (U.S. Pat. No. 4,376,110); the human B-cell hybridoma technique (Kozbor et al., 1984, J. Immunol. 133:3001-3005; Cole et al., 1983, Proc. Natl. Acad. Sci. USA 80:2026-2030); and the EBV-hybridoma technique (Cole et al., 1985, Monoclonal Antibodies And Cancer Therapy, Alan R. Liss, Inc., pp. 77-96). Hybridoma cell lines that produce monoclonal antibodies specific for the polypeptides of the invention are also contemplated herein. Such hybridomas can be produced and identified by conventional techniques. The hybridoma producing the mAb of this invention can be cultivated in vitro or in vivo. Production of high titers of mAbs in vivo makes this the presently preferred method of production. One method for producing such a hybridoma cell line comprises immunizing an animal with a polypeptide; harvesting spleen cells from the immunized animal; fusing said spleen cells to a myeloma cell line, thereby generating hybridoma cells; and identifying a hybridoma cell line that produces a monoclonal antibody that binds the polypeptide. For the production of antibodies, various host animals can be immunized by injection with one or more of the following: a RIPPA and/or a RIPPA-Like polypeptide, a fragment of a RIPPA or a RIPPA-Like polypeptide, a functional equivalent of a RIPPA and/or a RIPPA-Like polypeptide, or a mutant form of a RIPPA or a RIPPA-Like polypeptide. Such host animals can include but are not limited to rabbits, guinea pigs, mice, and rats. Various adjuvants can be used to increase the immunologic response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjutants such as BCG (bacille Calmette-Guerin) and Corynebacterium parvum. The monoclonal antibodies can be recovered by conventional techniques. Such monoclonal antibodies can be of any immunoglobulin class including IgG, IgM, IgE, IgA, IgD and any subclass thereof.

In addition, techniques developed for the production of “chimeric antibodies” (Takeda et al., 1985, Nature, 314: 452-454; Morrison et al., 1984, Proc Natl Acad Sci USA 81: 6851-6855; Boulianne et al., 1984, Nature 312: 643-646; Neuberger et al., 1985, Nature 314: 268-270) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a porcine mAb and a human immunoglobulin constant region. The monoclonal antibodies of the present invention also include humanized versions of murine monoclonal antibodies. Such humanized antibodies can be prepared by known techniques and offer the advantage of reduced immunogenicity when the antibodies are administered to humans. In one embodiment, a humanized monoclonal antibody comprises the variable region of a murine antibody (or just the antigen binding site thereof) and a constant region derived from a human antibody. Alternatively, a humanized antibody fragment can comprise the antigen binding site of a murine monoclonal antibody and a variable region fragment (lacking the antigen-binding site) derived from a human antibody. Procedures for the production of chimeric and further engineered monoclonal antibodies include those described in Riechmann et al. (Nature 332:323, 1988), Liu et al. (PNAS 84:3439, 1987), Larrick et al. (Bio/Technology 7:934, 1989), and Winter and Harris (TIPS 14:139, Can, 1993). Useful techniques for humanizing antibodies are also discussed in U.S. Pat. No. 6,054,297. Procedures to generate antibodies transgenically can be found in GB 2,272,440, U.S. Pat. Nos. 5,569,825 and 5,545,806, and related patents. Preferably, for use in humans, the antibodies are human or humanized; techniques for creating such human or humanized antibodies are also well known and are commercially available from, for example, Medarex Inc. (Princeton, N.J.) and Abgenix Inc. (Fremont, Calif.). In another preferred embodiment, fully human antibodies for use in humans are produced by screening a library of human antibody variable domains using either phage display methods (Vaughan et al., 1998, Nat Biotechnol. 16(6): 535-539; and U.S. Pat. No. 5,969,108), ribosome display methods (Schaffitzel et al., 1999, J Immunol Methods 231(1-2): 119-135), or mRNA display methods (Wilson et al., 2001, Proc Natl Acad Sci USA 98(7): 3750-3755).

Antigen-binding antibody fragments that recognize specific epitopes can be generated by known techniques. For example, such fragments include but are not limited to: the F(ab′)2 fragments which can be produced by pepsin digestion of the antibody molecule and the Fab fragments which can be generated by reducing the disulfide bridges of the (ab′)2 fragments. Alternatively, Fab expression libraries can be constructed (Huse et al., 1989, Science, 246:1275-1281) to allow rapid and easy identification of monoclonal Fab fragments with the desired specificity. Techniques described for the production of single chain antibodies (U.S. Pat. No. 4,946,778; Bird, 1988, Science 242:423-426; Huston et al., 1988, Proc. Natl. Acad. Sci. USA 85:5879-5883; and Ward et al., 1989, Nature 334:544-546) can also be adapted to produce single chain antibodies against RIPPA and/or RIPPA-Like gene products. Single chain antibodies are formed by linking the heavy and light chain fragments of the Fv region via an amino acid bridge, resulting in a single chain polypeptide. Such single chain antibodies can also be useful intracellularly (i.e., as intrabodies), for example as described by Marasco et al. (J. Immunol. Methods 231:223-238, 1999) for genetic therapy in HIV infection. In addition, antibodies to the RIPPA and/or RIPPA-Like polypeptide can, in turn, be utilized to generate anti-idiotype antibodies that “mimic” the RIPPA and/or RIPPA-Like polypeptide and that may bind to the RIPPA and/or RIPPA-Like polypeptide's binding partners using techniques well known to those skilled in the art. (See, e.g., Greenspan & Bona, 1993, FASEB J 7(5):437-444; and Nissinoff, 1991, J. Immunol. 147(8):2429-2438).

Antibodies that are immunoreactive with the polypeptides of the invention include bispecific antibodies (i.e., antibodies that are immunoreactive with the polypeptides of the invention via a first antigen binding domain, and also immunoreactive with a different polypeptide via a second antigen binding domain). A variety of bispecific antibodies have been prepared, and found useful both in vitro and in vivo (see, for, example, U.S. Pat. No. 5,807,706; and Cao and Suresh, 1998, Bioconjugate Chem 9: 635-644). Numerous methods of preparing bispecific antibodies are known in the art, including the use of hybrid-hybridomas such as quadromas, which are formed by fusing two differed hybridomas, and triomas, which are formed by fusing a hybridoma with a lymphocyte (Milstein and Cuello, 1983, Nature 305: 537-540; U.S. Pat. No. 4,474,893; and U.S. Pat. No. 6,106,833). U.S. Pat. No. 6,060,285 discloses a process for the production of bispecific antibodies in which at least the genes for the light chain and the variable portion of the heavy chain of an antibody having a first specificity are transfected into a hybridoma cell secreting an antibody having a second specificity. Chemical coupling of antibody fragments has also been used to prepare antigen-binding molecules having specificity for two different antigens (Brennan et al., 1985, Science 229: 81-83; Glennie et al., J. Immunol., 1987, 139:2367-2375; and U.S. Pat. No. 6,010,902). Bispecific antibodies can also be produced via recombinant means, for example, by using the leucine zipper moieties from the Fos and Jun proteins (which preferentially form heterodimers) as described by Kostelny et al. (J. Immnol. 148:1547-4553; 1992). U.S. Pat. No. 5,582,996 discloses the use of complementary interactive domains (such as leucine zipper moieties or other lock and key interactive domain structures) to facilitate heterodimer formation in the production of bispecific antibodies. Tetravalent, bispecific molecules can be prepared by fusion of DNA encoding the heavy chain of an F(ab′)2 fragment of an antibody with either DNA encoding the heavy chain of a second F(ab′)2 molecule (in which the CH1 domain is replaced by a CH3 domain), or with DNA encoding a single chain FV fragment of an antibody, as described in U.S. Pat. No. 5,959,083. Expression of the resultant fusion genes in mammalian cells, together with the genes for the corresponding light chains, yields tetravalent bispecific molecules having specificity for selected antigens. Bispecific antibodies can also be produced as described in U.S. Pat. No. 5,807,706. Generally, the method involves introducing a protuberance (constructed by replacing small amino acid side chains with larger side chains) at the interface of a first polypeptide and a corresponding cavity (prepared by replacing large amino acid side chains with smaller ones) in the interface of a second polypeptide. Moreover, single-chain variable fragments (sFvs) have been prepared by covalently joining two variable domains; the resulting antibody fragments can form dimers or trimers, depending on the length of a flexible linker between the two variable domains (Kortt et al., 1997, Protein Engineering 10:423-433).

Screening procedures by which such antibodies can be identified are well known, and can involve immunoaffinity chromatography, for example. Antibodies can be screened for agonistic (i.e., ligand-mimicking) properties. Such antibodies, upon binding to cell surface portions of RIPPA and/or RIPPA-Like polypeptides, induce biological effects (e.g., transduction of biological signals) similar to the biological effects induced when the RIPPA and/or RIPPA-Like binding partner binds to RIPPA and/or RIPPA-Like polypeptides a Agonistic antibodies can be used to induce RIPPA- and/or RIPPA-Like-mediated cell stimulatory pathways or intercellular communication. Bispecific antibodies can be identified by screening with two separate assays, or with an assay wherein the bispecific antibody serves as a bridge between the first antigen and the second antigen (the latter is coupled to a detectable moiety). Bispecific antibodies that bind RIPPA and/or RIPPA-Like polypeptides of the invention via a first antigen binding domain will be useful in diagnostic applications and in treating osteoporosis, osteomyelitis, hypercalcemia, osteopenia brought on by surgery or steroid administration, prosthetic loosening, Paget's disease, osteonecrosis, bone loss due to rheumatoid arthritis, periodontal bone loss, and cancers that may metastasize to bone and induce bone breakdown, such as multiple myeloma, breast cancer and some melanomas and related conditions.

Those antibodies that can block binding of the RIPPA and/or RIPPA-Like polypeptides of the invention to binding partners for RIPPA and/or RIPPA-Like polypeptides can be used to inhibit RIPPA- and/or RIPPA-Like-mediated intercellular communication or cell stimulation that results from such binding. Such blocking antibodies can be identified using any suitable assay procedure, such as by testing antibodies for the ability to inhibit binding of RIPPA and/or RIPPA-Like to certain binding partners. Antibodies can be assayed for the ability to inhibit RIPPA and/or RIPPA-Like binding partner-mediated cell stimulatory pathways, for example. Such an antibody can be employed in an in vitro procedure, or administered in vivo to inhibit a biological activity mediated by the entity that generated the antibody. Disorders caused or exacerbated (directly or indirectly) by the interaction of RIPPA and/or RIPPA-Like with cell surface binding partner receptor thus can be treated. A therapeutic method involves in vivo administration of a blocking antibody to a mammal in an amount effective in inhibiting RIPPA and/or RIPPA-Like binding partner-mediated biological activity. Monoclonal antibodies are generally preferred for use in such therapeutic methods. In one embodiment, an antigen-binding antibody fragment is employed. Compositions comprising an antibody that is directed against RIPPA or a RIPPA-Like polypeptide, and a physiologically acceptable diluent, excipient, or carrier, are provided herein. Suitable components of such compositions are as described below for compositions containing RIPPA and/or RIPPA-Like polypeptides.

Also provided herein are conjugates comprising a detectable (e.g., diagnostic) or therapeutic agent, attached to the antibody. Examples of such agents are presented above. The conjugates find use in in vitro or in vivo procedures. The antibodies of the invention can also be used in assays to detect the presence of the polypeptides or fragments of the invention, either in vitro or in vivo. The antibodies also can be employed in purifying polypeptides or fragments of the,invention by immunoaffinity chromatography.

Examples of assays that may be used to screen agonistic or antagonistic antibodies are described below.

Rational Design of Compounds that Interact with RIPPA and/or RIPPA-Like Polypeptides

The goal of rational drug design is to produce structural analogs of biologically active polypeptides of interest or of small molecules with which they interact, e.g., inhibitors, agonists, antagonists, etc. Any of these examples can be used to fashion drugs which are more active or stable forms of the polypeptide or which enhance or interfere with the function of a polypeptide in vivo (Hodgson J (1991) Biotechnology 9:19-21). In one approach, the three-dimensional structure of a polypeptide of interest, or of a polypeptide-inhibitor complex, is determined by x-ray crystallography, by nuclear magnetic resonance, or by computer homology modeling or, most typically, by a combination of these approaches. Both the shape and charges of the polypeptide must be ascertained to elucidate the structure and to determine active site(s) of the molecule. Less often, useful information regarding the structure of a polypeptide may be gained by modeling based on the structure of homologous polypeptides. In both cases, relevant structural information is used to design analogous molecules or RIPPA and/or RIPPA-Like polypeptides, to identify efficient inhibitors, or to identify small molecules that bind RIPPA and/or RIPPA-Like polypeptides. Useful examples of rational drug design include molecules which have improved activity or stability as shown by Braxton S and Wells J A (1992 Biochemistry 31:7796-7801) or which act as inhibitors, agonists, or antagonists of native peptides as shown by Athauda S B et al (1993 J Biochem 113:742-746). The use of RIPPA and/or RIPPA-Like polypeptide structural information in molecular modeling software systems to assist in inhibitor design and in studying inhibitor-RIPPA and/or RIPPA-Like polypeptide interaction is also encompassed by the invention. A particular method of the invention comprises analyzing the three dimensional structure of RIPPA and/or RIPPA-Like polypeptides for likely binding sites of substrates, synthesizing a new molecule that incorporates a predictive reactive site, and assaying the new molecule as described further herein.

It is also possible to isolate a target-specific antibody, selected by functional assay, as described further herein, and then to solve its crystal structure. This approach, in principle, yields a pharmacore upon which subsequent drug design can be based. It is possible to bypass polypeptide crystallography altogether by generating anti-idiotypic antibodies (anti-ids) to a functional, pharmacologically active antibody. As a mirror image of a mirror image, the binding site of the anti-ids would be expected to be an analog of the original antigen. The anti-id could then be used to identify and isolate peptides from banks of chemically or biologically produced peptides. The isolated peptides would then act as the pharmacore.

Assays of RIPPA and/or RIPPA-Like Polypeptide Activities

The purified RIPPA and RIPPA-Like polypeptides of the invention (including polypeptides, fragments, variants, oligomers, and other forms) are useful in a variety of assays. For example, the RIPPA and RIPPA-Like molecules of the present invention can be used to identify binding partners of RIPPA and/or RIPPA-Like polypeptides, which can also be used to modulate intercellular communication, cell stimulation, or immune cell activity. Alternatively, they can be used to identify non-binding-partner molecules or substances that modulate intercellular communication, cell stimulatory pathways, or immune cell activity.

Assays for determining the effects of RIPPA and/or RIPPA-Like agonists and antagonists include, but are not limited to, culturing monocyte/macrophage cells in vitro and inducing said cells to mature/differentiate to osteoclasts. These cells are cultured on bone or dentin whereby the osteoclasts excavate resorptive lacunae in the bone or dentin substrate. Alternatively, the cells may be cultured on calcium phophate matrices, such as those described in Langstaff, S., et al, Biomaterials, January 2001;22(2): 135-50 or commercially available from sources such as the Biosciences Division of Bectin Dickinson, Frannklin Lakes, N.J. The number and size of resorption lacunae formed in the bone, dentin or calcium phophate substrate are a quantitative measure of osteoclast activity (see, Fuller, K., et al. J. Bone Miner. Res. 1994, 9:17). RIPPA and/or RIPPA-Like agonists or antagonists may be added to the assay at any time point to determine if the RIPPA and/or RIPPA-Like agonists or antagonists cause an increase or decrease in the relative number and/or size of resorption lacunae. A quantitative measurement of the number and/or size of resorption lacunae formed in the bone, dentin or calcium phosphate substrate is performed using standard techniques to ascertain differences between cultures receiving a RIPPA and/or RIPPA-Like agonists or antagonists and those that did not. Cultures receiving RIPPA and/or RIPPA-Like agonists or antagonists that have an increased or decreased relative number and/or size of resorption lacunae indicate a potential candidate for therapeutic use in modulating RIPPA and/or RIPPA-Like activities. Additional in vitro screening assays for osteoclast activity may be used to screen for RIPPA and/or RIPPA-Like agonists and/or antagonitsts, such as those using biotinylated bone, dentin or calcium phosphate substrates, as described in Nesbitt, S. A., et al. 1997, Science 276:266.

In vitro screening assay may be used to determine the effects of RIPPA and/or RIPPA-Like agonists and/or antagonitsts on osteoclastogenesis. Biological models of osteoclast differentiation have been developed that facilitate the detailed study the factors involved in the regulation of this process. One embodiment comprises cultures of mouse bone marrow or cocultures of haematopoietic cells with bone-derived stromal cells, which give rise to large numbers of bone-resorbing oseoclasts. One of skill in the art would be familiar with such, assays, such as those described in Rodman, G. D., Experimental Hematology 1999, 27: 1229-1241; Suda, T., et al. Endocr Rev 1992, 13:66-80; Takahashi, N., et al. Endocrinology 1998, 123:2600-2602; Quinn, J. M., et al. Endocrinology 1994, 134:2416-2423; Kurihara, N., et al. J Bone Miner Res 1991, 6:257-261; Matayoshi, A., et al. PNAS 1996, 93:10785-10790; and, Roux, S., et al. J Cell Physiol 1996, 168:489-498. In alternative embodiments, in vitro screening assays include culturing established monocyte/macrophage cell lines or primary monocyte cultures, and inducing said cells to mature/differentiate to osteoclasts, as described in detail in Examples 4 and 5.

Additional readouts may be used to determine the effects of RIPPA and/or RIPPA-Like agonists and/or antagonists in the in vitro assays described above, such as monitoring surrogate markers of osteoclastogenesis or osteoclastic bone resorption. For example, the TRAP (Tartrate-Resistant Acid Phosphatase) assay may be used to monitor tartrate-resistant acid phosphatase (also referred to as type 5 acid phosphatase), which is a marker enzyme of bone-resorbing osteoclasts. Cells from the assays described above may also be stained for TRAP using standard histological or immunohistochemical techniques. In alternative assays, culture or cellular levels of osteocalcin may be monitored as an indicator of recently formed bone. Osteoclacin is a protein specifically produced by osteoblasts and is an integral component of bone formation; commercial kits are available, but any suitable method or assay for measuring culture or cellular levels of osteocalcin may be used.

In alternative embodiments, reporter assays may be used to measure the effects of RIPPA and/or RIPPA-Like agonists and/or antagonists on osteoclastogenesis or osteoclastic bone resorption in in vitro assays. Reporter assays are well known in the art and are readily amenable to this analysis. Examples include, but are not limited to, analytical chemiluminescence and bioluminescence, expression of cell surface molecules, expression and release of soluble biomolecules, and the like. In one embodiment a construct is created comprising the mmP9 promoter operably linked to polynucleotide sequences encoding a cytokine, such as IL-2. This contruct is transfected into osteoclast precursors, such as the RAW 264.7 macrophage cell line. Upon stimulation by one or more factors that cause the cells to differentiate into osteoclasts, such as exposure to RANK-L, the mmP9 promoter is activated and IL-2 is released as the reporter and is indicative of osteoclastogenesis and/or osteclastogenic activity.

RIPPA and/or RIPPA-Like agonists and/or antagonists may be tested in vivo. Bone collagens are extensively degraded by the action of collagenolytic enzymes during resorption and the resultant release of type I collagen fragments into the extracellular space may be detected in the plasma and urine, thereby providing a clinical measurement of bone resorption. RIPPA and/or RIPPA-Like agonists and/or antagonists are administered to a subject and relative amounts of type I collagen in the plasma and/or urine are measured. Subjects receiving RIPPA and/or RIPPA-Like agonists or antagonists that have an increase or decrease in the relative amount of type I collagen in the plasma and/or urine indicate a potential candidate for therapeutic use in modulating RIPPA and/or RIPPA-Like activities. Additional assays for monitoring in vivo testing of RIPPA and/or RIPPA-Like agonists and/or antagonists include the TRAP assay (commercially available assays include BoneTRAP® assay (Suomen Bioanalytiikka Oy, Turku, Finland). In general, TRAP, tartrate-resistant acid phosphatase, is secreted into the circulation by osteoclasts and it has been shown that circulating serum levels of TRAP is a useful marker of bone resorption activity. In alternative assays, serum levels of osteocalcin may be monitored as an indicator of recently formed bone. Osteoclacin is a protein specifically produced by osteoblasts and is an integral component of bone formation. Commercial kits are available, such as the IMMULITE® Osteocalcin Assay (Diagnotic Products Corp., Los Angeles, Calif.), but any suitable method or assay for measuring serum levels of osteocalcin may be used.

In alternative in vivo assays, Dual-energy X-ray Absorptiometry (DEXA) for bone mineral density determination is well-established in the art and may be used to assess the qualitative and quantitative differences between subjects receiving RIPPA and/or RIPPA-Like agonists or antagonists and subjects that did not. Additional embodiments of monitoring bone density and therefore the efficacy of RIPPA and/or RIPPA-Like agonists or antagonists include for example, single-photon absorptiometry, dual-photon absorptiometry, quantitative computed tomography and radiographic absorptiometry. Also, traditional pathological, histological and/or immunohistochemical staining of tissues may be used to assess pathological differences between study groups.

Assays to Identify Binding Partners. Polypeptides of the RIPPA and/or RIPPA-Like family and fragments thereof can be used to identify binding partners. For example, they can be tested for the ability to bind a candidate binding partner in any suitable assay, such as a conventional binding assay. To illustrate, the RIPPA and/or RIPPA-Like polypeptide can be labeled with a detectable reagent (e.g., a radionuclide, chromophore, enzyme that catalyzes a colorimetric or fluorometric reaction, and the like). The labeled polypeptide is contacted with cells expressing the candidate binding partner. The cells then are washed to remove unbound labeled polypeptide, and the presence of cell-bound label is determined by a suitable technique, chosen according to the nature of the label.

One example of a binding assay procedure is as follows. A recombinant expression vector containing the candidate binding partner cDNA is constructed. CV1-EBNA-1 cells in 10 cm² dishes are transfected with this recombinant expression vector. CV-1/EBNA-1 cells (ATCC CRL 10478) constitutively express EBV nuclear antigen-1 driven from the CMV Immediate-early enhancer/promoter. CV1-EBNA-1was4 derived from the African Green Monkey kidney cell line CV-1 (ATCC CCL 70), as described by McMahan et al., (EMBO J. 10:2821, 1991). The transfected cells are cultured for 24 hours, and the cells in each dish then are split into a 24-well plate. After culturing an additional 48 hours, the transfected cells (about 4×10⁴ cells/well) are washed with BM-NFDM, which is binding medium (RPMI 1640 containing 25 mg/ml bovine serum albumin, 2 mg/nl sodium azide, 20 mM Hepes pH 7.2) to which 50 mg/ml nonfat dry milk has been added. The cells then are incubated for 1 hour at 37° C. with various concentrations of, for example, a soluble polypeptide/Fc fusion polypeptide made as set forth above. Cells then are washed and incubated with a constant saturating concentration of a ¹²⁵I-mouse anti-human IgG in binding medium, with gentle agitation for 1 hour at 37° C. After extensive washing, cells are released via trypsinization. The mouse anti-human IgG employed above is directed against the Fc region of human IgG and can be obtained from Jackson Immunoresearch Laboratories, Inc., West Grove, Pa. The antibody is radioiodinated using the standard chloramine-T method. The antibody will bind to the Fc portion of any polypeptide/Fc polypeptide that has bound to the cells. In all assays, non-specific binding of ¹²⁵I-antibody is, assayed in the absence of the Fc fusion polypeptide/Fc, as well as in the presence of the Fc fusion polypeptide and a 200-fold molar excess of unlabeled mouse anti-human IgG antibody. Cell-bound ¹²⁵I-antibody is quantified on a Packard Autogamma counter. Affinity calculations (Scatchard, Ann. N.Y. Acad. Sci. 51:660, 1949) are generated on RS/1 (BBN Software, Boston, Mass.) run on a Microvax computer. Binding can also be detected using methods that are well suited for high-throughput screening procedures, such as scintillation proximity assays (Udenfriend et al., 1985, Proc Natl Acad Sci USA 82: 8672-8676), homogeneous time-resolved fluorescence methods (Park et al., 1999, Anal Biochem 269: 94-104), fluorescence resonance energy transfer (FRET) methods (Clegg R M, 1995, Curr Opin Biotechnol 6: 103-110), or methods that measure any changes in surface plasmon resonance when a bound polypeptide is exposed to a potential binding partner, using for example a biosensor such as that supplied by Biacore AB (Uppsala, Sweden). Compounds that can be assayed for binding to RIPPA and/or RIPPA-Like polypeptides include but are not limited to small organic molecules, such as those that are commercially available—often as part of large combinatorial chemistry compound ‘libraries’—from companies such as Sigma-Aldrich (St. Louis, Mo.), Arqule (Woburn, Mass.), Enzymed (Iowa City, Iowa), Maybridge Chemical Co.(Trevillett, Cornwall, UK), MDS Panlabs (Bothell, Wash.), Pharmacopeia (Princeton, N.J.), and Trega (San Diego, Calif.). Preferred small organic molecules for screening using these assays are usually less than 10K molecular weight and can possess a number of physicochemical and pharmacological properties which enhance cell penetration, resist degradation, and/or prolong their physiological half-lives (Gibbs, J., 1994, Pharmaceutical Research in Molecular Oncology, Cell 79(2): 193-198). Compounds including natural products, inorganic chemicals, and biologically active materials such as proteins and toxins can also be assayed using these methods for the ability to bind to RIPPA and/or RIPPA-Like polypeptides.

Yeast Two-Hybrid or “Interaction Trap” Assays. Where the RIPPA and/or RIPPA-Like polypeptide binds or potentially binds to another polypeptide (such as, for example, in a receptor-ligand interaction), the nucleic acid encoding the RIPPA or RIPPA-Like polypeptide can also be used in interaction trap assays (such as, for example, that described in Gyuris et al., Cell 75:791-803 (1993)) to identify nucleic acids encoding the other polypeptide with which binding occurs or to identify inhibitors of the binding interaction. Polypeptides involved in these binding interactions can also be used to screen for peptide or small molecule inhibitors or agonists of the binding interaction.

Competitive Binding Assays. Another type of suitable binding assay is a competitive binding assay. To illustrate, biological activity of a variant can be determined by assaying for the variant's ability to compete with the native polypeptide for binding to the candidate binding partner. Competitive binding assays can be performed by conventional methodology. Reagents that can be employed in competitive binding assays include radiolabeled RIPPA and/or RIPPA-Like and intact cells expressing portions of RIPPA and/or RIPPA-Like (endogenous or recombinant) polypeptides on the cell surface. For example, a radiolabeled soluble RIPPA or RIPPA-Like fragment can be used to compete with a soluble RIPPA and/or RIPPA Like variant for binding to binding partners. A soluble binding partner/Fc fusion polypeptide bound to a solid phase through the interaction of Polypeptide A or Polypeptide G (on the solid phase) with the Fc moiety can be used. Chromatography columns that contain Polypeptide A and Polypeptide G include those available from Pharmacia Biotech, Inc., Piscataway, N.J.

Assays to Identify Modulators of Intercellular Communication, Cell Stimulation, or Immune Cell Activity. The influence of RIPPA and/or RIPPA-Like polypeptides on intercellular communication, cell stimulation, or immune cell activity can be manipulated to control these activities in target cells. For example, the disclosed RIPPA and/or RIPPA-Like polypeptides, nucleic acids encoding the disclosed RIPPA and/or RIPPA-Like polypeptides, or agonists or antagonists of such polypeptides can be administered to a cell or group of cells to induce, enhance, suppress, or arrest cellular communication, cell stimulation, or activity in the target cells. Identification of RIPPA and/or RIPPA-Like polypeptides, agonists or antagonists that can be used in this manner can be carried out via a variety of assays known to those skilled in the art. Included in such assays are those that evaluate the ability of an RIPPA and/or RIPPA-Like polypeptide to influence intercellular communication, cell stimulation or activity. Such an assay would involve, for example, the analysis of immune cell interaction in the presence of an RIPPA and/or RIPPA-Like polypeptide. In such an assay, one would determine a rate of communication or cell stimulation in the presence of the RIPPA and/or RIPPA-Like polypeptide and then determine if such communication or cell stimulation is altered in the presence of a candidate agonist or antagonist or another RIPPA and/or RIPPA-Like polypeptide. Exemplary assays for this aspect of the invention include cytokine secretion assays, T-cell co-stimulation assays and mixed lymphocyte reactions involving antigen presenting cells and T cells. These assays are well known to those skilled in the art.

In another aspect, the present invention provides a method of detecting the ability of a test compound to affect the intercellular communication or cell stimulatory activity of a cell. In this aspect, the method comprises: (1) contacting a first group of target cells with a test compound including an RIPPA and/or RIPPA-Like receptor polypeptide or fragment thereof under conditions appropriate to the particular assay being used; (2) measuring the net rate of intercellular communication or cell stimulation among the target cells; and (3) observing the net rate of intercellular communication or cell stimulation among control cells containing the RIPPA and/or RIPPA-Like receptor polypeptides or fragments thereof, in the absence of a test compound, under otherwise identical conditions as the first group of cells. In this embodiment, the net rate of intercellular communication or cell stimulation in the control cells is compared to that of the cells treated with both the RIPPA and/or RIPPA-Like molecule as well as a test compound. The comparison will provide a difference in the net rate of intercellular communication or cell stimulation such that an effector of intercellular communication or cell stimulation can be identified. The test compound can function as an effector by either activating or up-regulating, or by inhibiting or down-regulating intercellular communication or cell stimulation, and can be detected through this method.

Cell Proliferation, Cell Death, Cell Differentiation, and Cell Adhesion Assays. A polypeptide of the present invention may exhibit cytokine, cell proliferation (either inducing or inhibiting), or cell differentiation (either inducing or inhibiting) activity, or may induce production of other cytokines in certain cell populations. Many polypeptide factors discovered to date have exhibited such activity in one or more factor-dependent cell proliferation assays, and hence the assays serve as a convenient confirmation of cell stimulatory activity. The activity of a polypeptide of the present invention is evidenced by any one of a number of routine factor-dependent cell proliferation assays for cell lines including, without limitation, 32D, DA2, DA1G, T10, B9, B9/11, BaF3, MC9/G, M+ (preB M+), 2E8, RB5, DA1, 123, T1165, HT2, CTLL2, TF-1, Mo7e and CMK. The activity of a RIPPA and/or RIPPA-Like polypeptide of the invention may, among other means, be measured by the following methods:

Assays for T-cell or thymocyte proliferation include without limitation those described in: Current Protocols in Immunology, Coligan et al. eds, Greene Publishing Associates and Wiley-Interscience (pp. 3.1-3.19: In vitro assays for mouse lymphocyte function; Chapter 7: Immunologic studies in humans); Takai et al., J. Immunol. 137: 3494-3500, 1986; Bertagnolli et al., J. Immunol. 145: 1706-1712, 1990; Bertagnolli et al., Cellular Immunology 133:327-341, 1991; Bertagnolli, et al., J. Immunol. 149:3778-3783, 1992; Bowman et al., J. Immunol. 152: 1756-1761, 1994.

Assays for cytokine production and/or proliferation of spleen cells, lymph node cells or thymocytes include, without limitation, those described in: Kruisbeek and Shevach, 1994, Polyclonal T cell stimulation, in Current Protocols in Immunology, Coligan et al. eds. Vol 1 pp. 3.12.1-3.12.14, John Wiley and Sons, Toronto; and Schreiber, 1994, Measurement of mouse and human interferon gamma in Current Protocols in Immunology, Coligan et al. eds. Vol 1 pp. 6.8.1-6.8.8, John Wiley and Sons, Toronto.

Assays for proliferation and differentiation of hematopoietic and lymphopoietic cells include, without limitation, those described in: Bottomly et al., 1991, Measurement of human and murine interleukin 2 and interleukin 4, in Current Protocols in Immunology, Coligan et al. eds. Vol 1 pp. 6.3.1-6.3.12, John Wiley and Sons, Toronto; deVries et al., J Exp Med 173: 1205-1211, 1991; Moreau et al., Nature 336:690-692, 1988; Greenberger et al., Proc Natl Acad Sci. USA 80: 2931-2938, 1983; Nordan, 1991, Measurement of mouse and human interleukin 6, in Current Protocols in Immunology Coligan et al. eds. Vol 1 pp. 6.6.1-6.6.5, John Wiley and Sons, Toronto; Smith et al., Proc Natl Acad Sci USA 83: 1857-1861, 1986; Bennett et al., 1991, Measurement of human interleukin 11, in Current Protocols in Immunology Coligan et al. eds. Vol 1 pp. 6.15.1 John Wiley and Sons, Toronto; Ciarletta et al., 1991, Measurement of mouse and human Interleukin 9, in Current Protocols in Immunology Coligan et al. eds. Vol 1 pp. 6.13.1, John Wiley and Sons, Toronto.

Assays for T-cell clone responses to antigens (which will identify, among others, polypeptides that affect APC-T cell interactions as well as direct T-cell effects by measuring proliferation and cytokine production) include, without limitation, those described in: Current Protocols in Immunology, Coligan et al. eds, Greene Publishing Associates and Wiley-Interscience (Chapter 3: In vitro assays for mouse lymphocyte function; Chapter 6: Cytokines and their cellular receptors; Chapter 7: Immunologic studies in humans); Weinberger et al., Proc Natl Acad Sci USA 77: 6091-6095, 1980; Weinberger et al., Eur. J. Immun. 11:405-411, 1981; Takai et al., J. Immunol. 137:3494-3500, 1986; Takai et al., J. Immunol. 140:508-512, 1988

Assays for thymocyte or splenocyte cytotoxicity include, without limitation, those described in: Current Protocols in Immunology, Coligan et al. eds, Greene Publishing Associates and Wiley-Interscience (Chapter 3, In Vitro assays for Mouse Lymphocyte Function 3.1-3.19; Chapter 7, Immunologic studies in Humans); Herrmann et al., Proc. Natl. Acad. Sci., USA 78:2488-2492, 1981; Herrmann et al., J. Immunol. 128:1968-1974, 1982; Handa et al., J. Immunol. 135:1564-1572, 1985; Takai et al., J. Immunol. 1137:3494-3500, 1986; Takaiet al., J. Immunol. 140:508-512, 1988; Herrmann et al., Proc. Natl. Acad. Sci. USA 78:2488-2492, 1981; Herrmann et al., J. Immunol. 128:1968-1974, 1982; Handa et al. J. Immunol. 135:1564-1572, 1985; Takai et al., J. Immunol. 137:3494-3500, 1986; Bowmanet al., J. Virology 61:1992-1998; Takai et al., J. Immunol. 140:508-512, 1988; Bertagnolli et al., Cellular Immunology 133:327-341, 1991; Brown et al., J. Immunol. 153:3079-3092, 1994.

Assays for T-cell-dependent immunoglobulin responses and isotype switching (which will identify, among others, polypeptides that modulate T-cell dependent antibody responses and that affect Th1/Th2 profiles) include, without limitation, those described in: Maliszewski, J Immunol 144: 3028-3033, 1990; and Mond and Brunswick, 1994, Assays for B cell function: in vitro antibody production, in Current Protocols in Immunology Coligan et al. eds. Vol 1 pp. 3.8.1-3.8.16, John Wiley and Sons, Toronto.

Mixed lymphocyte reaction (MLR) assays (which will identify, among others, polypeptides that generate predominantly Th1 and CTL responses) include, without limitation, those described in: Current Protocols in Immunology, Coligan et al. eds, Greene Publishing Associates and Wiley-Interscience (Chapter 3, In Vitro assays for Mouse Lymphocyte Function 3.1-3.19; Chapter 7, Immunologic studies in Humans); Takai et al., J. Immunol. 137:3494-3500, 1986; Takai et al., J. Immunol. 140:508-512, 1988; Bertagnolli et al., J. Immunol. 149:3778-3783, 1992.

Dendritic cell-dependent assays (which will identify, among others, polypeptides expressed by dendritic cells that activate naive T-cells) include, without limitation, those described in: Guery et al., J. Immunol 134:536-544, 1995; Inaba et al., J Exp Med 173:549-559, 1991; Macatonia et al., J Immunol 154:5071-5079, 1995; Porgador et al., J Exp Med 182:255-260, 1995; Nair et al., J Virology 67:4062-4069, 1993; Huang et al., Science 264:961-965, 1994; Macatonia et al., J Exp Med 169:1255-1264, 1989; Bhardwaj et al., J Clin Invest 94:797-807, 1994; and Inaba et al., J Exp Med 172:631-640,1990.

Assays for lymphocyte survival/apoptosis (which will identify, among others, polypeptides that prevent apoptosis after superantigen induction and polypeptides that regulate lymphocyte homeostasis) include, without limitation, those described in: Darzynkiewicz et al., Cytometry 13:795-808, 1992; Gorczyca et al., Leukemia 7:659-670, 1993; Gorczyca et al., Cancer Research 53:1945-1951, 1993; Itoh et al., Cell 66:233-243, 1991; Zacharchuk, J Immunol 145:4037-4045, 1990; Zamai et al., Cytometry 14:891-897, 1993; Gorczyca et al., International Journal of Oncology 1:639-648, 1992.

Assays for polypeptides that influence early steps of T-cell commitment and development include, without limitation, those described in: Antica et al., Blood 84:111-117, 1994; Fine et al., Cell Immunol 155:111-122, 1994; Galy et al., Blood 85:2770-2778, 1995; Toki et al., Proc Natl Acad Sci. USA 88:7548-7551, 1991

Assays for embryonic stem cell differentiation (which will identify, among others, polypeptides that influence embryonic differentiation hematopoiesis) include, without limitation, those described in: Johansson et al. Cellular Biology 15:141-151, 1995; Keller et al., Molecular and Cellular Biology 13:473-486, 1993; McClanahan et al., Blood 81:2903-2915, 1993.

Assays for stem cell survival and differentiation (which will identify, among others, polypeptides that regulate lympho-hematopoiesis) include, without limitation, those described in: Methylcellulose colony forming assays, Freshney, 1994, In Culture of Hematopoietic Cells, Freshney et al. eds. pp. 265-268, Wiley-Liss, Inc., New York, N.Y.; Hirayama et al., Proc. Natl. Acad. Sci. USA 89:5907-5911, 1992; Primitive hematopoietic colony forming cells with high proliferative potential, McNiece and Briddell, 1994, In Culture of Hematopoietic Cells, Freshney et al. eds. pp. 23-39, Wiley-Liss, Inc., New York, N.Y.; Neben et al., Experimental Hematology 22:353-359, 1994; Ploemacher, 1994, Cobblestone area forming cell assay, In Culture of Hematopoietic Cells, Freshney et al. eds. pp. 1-21, Wiley-Liss, Inc., New York, N.Y.; Spooncer et al., 1994, Long term bone marrow cultures in the presence of stromal cells, In Culture of Hematopoietic Cells, Freshney et al. eds. pp. 163-179, Wiley-Liss, Inc., New York, N.Y.; Sutherland, 1994, Long term culture initiating cell assay, In Culture of Hematopoietic Cells, Freshney et al. eds. Vol pp. 139-162, Wiley-Liss, Inc., New York, N.Y.

Assays for tissue generation activity include, without limitation, those described in: International Patent Publication No. WO95/16035 (bone, cartilage, tendon); International Patent Publication No. WO95/05846 (nerve, neuronal); International Patent Publication No. WO91/07491 (skin, endothelium). Assays for wound healing activity include, without limitation, those described in: Winter, Epidermal Wound Healing, pps. 71-112 (Maibach and Rovee, eds.), Year Book Medical Publishers, Inc., Chicago, as modified by Eaglstein and Mertz, J. Invest. Dermatol 71:382-84 (1978).

Assays for activin/inhibin activity include, without limitation, those described in: Vale et al., Endocrinology 91:562-572, 1972; Ling et al., Nature 321:779-782, 1986; Vale et al., Nature 321:776-779, 1986; Mason et al., Nature 318:659-663, 1985; Forage et al., Proc. Natl. Acad. Sci. USA 83:3091-3095, 1986.

Assays for cell movement and adhesion include, without limitation, those described in: Current Protocols in Immunology Coligan et al. eds, Greene Publishing Associates and Wiley-Interscience (Chapter 6.12, Measurement of alpha and beta chemokines 6.12.1-6.12.28); Taub et al. J. Clin. Invest. 95:1370-1376, 1995; Lind et al. APMIS 103:140-146, 1995; Muller et al Eur. J. Immunol. 25: 1744-1748; Gruber et al. J Immunol. 152:5860-5867, 1994; Johnston et al. J Immunol. 153: 1762-1768, 1994

Assay for hemostatic: and thrombolytic activity include, without limitation, those described in: Linet et al., J. Clin. Pharmacol. 26:131-140, 1986; Burdick et al., Thrombosis Res. 45:413-419,1987; Humphrey et al., Fibrinolysis 5:71-79 (1991); Schaub, Prostaglandins 35:467-474, 1988.

Assays for receptor-ligand activity include without limitation those described in: Current Protocols in Immunology Coligan et al. eds, Greene Publishing Associates and Wiley-Interscience (Chapter 7.28, Measurement of cellular adhesion under static conditions 7.28.1-7.28.22), Takai et al., Proc. Natl. Acad. Sci. USA 84:6864-6868, 1987; Bierer et al., J. Exp. Med. 168:1145-1156, 1988; Rosenstein et al., J. Exp. Med. 169:149-160 1989; Stoltenborg et al., J. Immunol. Methods 175:59-68, 1994; Stitt et al., Cell 80:661-670, 1995.

Assays for cadherin adhesive and invasive suppressor activity include, without limitation, those described in: Hortsch et al. J Biol Chem 270 (32): 18809-18817, 1995; Miyaki et al. Oncogene 11: 2547-2552, 1995; Ozawa et al. Cell 63:1033-1038, 1990.

Diagnostic and Other Uses of RIPPA and/or RIPPA-Like Polypeptides and Nucleic Acids

The nucleic acids encoding the RIPPA and RIPPA-Like polypeptides provided by the present invention can be used for numerous diagnostic or other useful purposes. The nucleic acids of the invention can be used to express recombinant polypeptide for analysis, characterization or therapeutic use; as markers for tissues in which the corresponding polypeptide is preferentially expressed (either constitutively or at a particular stage of tissue differentiation or development or in disease states); as molecular weight markers on Southern gels; as chromosome markers or tags (when labeled) to identify chromosomes or to map related gene positions; to compare with endogenous DNA sequences in patients to identify potential genetic disorders; as probes to hybridize and thus discover novel, related DNA sequences; as a source of information to derive PCR primers for genetic fingerprinting; as a probe to “subtract-out” known sequences in the process of discovering other novel nucleic acids; for selecting and making oligomers for attachment to a “gene chip” or other support, including for examination of expression patterns; to raise anti-polypeptide antibodies using DNA immunization techniques; as an antigen to raise anti-DNA antibodies or elicit another immune response, and for gene therapy. Uses of RIPPA and RIPPA-Like polypeptides and fragmented polypeptides include, but are not limited to, the following: purifying polypeptides and measuring the activity thereof; delivery agents; therapeutic and research reagents; molecular weight and isoelectric focusing markers; controls for peptide fragmentation; identification of unknown polypeptides; and preparation of antibodies. Any or all nucleic acids suitable for these uses are capable of being developed into reagent grade or kit format for commercialization as products. Methods for performing the uses listed above are well known to those skilled in the art. References disclosing such methods include without limitation “Molecular Cloning: A Laboratory Manual”, 2d ed., Cold Spring Harbor Laboratory Press, Sambrook, J. E. F. Fritsch and T. Maniatis eds., 1989, and “Methods in Enzymology: Guide to Molecular Cloning Techniques”, Academic Press, Berger, S. L. and A. R. Kimmel eds.; 1987.

Probes and Primers. Among the uses of the disclosed RIPPA and RIPPA-Like nucleic acids, and combinations of fragments thereof, is the use of fragments as probes or primers. Such fragments generally comprise at least about 17 contiguous nucleotides of a DNA sequence. In other embodiments, a DNA fragment comprises at least 30, or at least 60, contiguous nucleotides of a DNA sequence. The basic parameters affecting the choice of hybridization conditions and guidance for devising suitable conditions are set forth by Sambrook et al., 1989 and are described in detail above. Using knowledge of the genetic code in combination with the amino acid sequences set forth above, sets of degenerate oligonucleotides can be prepared. Such oligonucleotides are useful as primers, e.g., in polymerase chain reactions (PCR), whereby DNA fragments are isolated and amplified. In certain embodiments, degenerate primers can be used as probes for non-human genetic libraries. Such libraries would include but are not limited to cDNA libraries, genomic libraries, and even electronic EST (express sequence tag) or DNA libraries. Homologous sequences identified by this method would then be used as probes to identify non-human RIPPA and/or RIPPA-Like homologues.

Chromosome Mapping. The nucleic acids encoding RIPPA and RIPPA-Like polypeptides, and the disclosed fragments and combinations of these nucleic acids, can be used by those skilled in the art using well-known techniques to identify the human chromosome to which these nucleic acids map. Useful techniques include, but are not limited to, using the sequence or portions, including oligonucleotides, as a probe in various well-known techniques such as radiation hybrid mapping (high resolution), in situ hybridization to chromosome spreads (moderate resolution), and Southern blot hybridization to hybrid cell lines containing individual human chromosomes (low resolution). For example, chromosomes can be mapped by radiation hybridization. PCR is performed using the Whitehead Institute/MIT Center for Genome Research Genebridge4 panel of 93 radiation hybrids, using primers that lie within a putative exon of the gene of interest and which amplify a product from human genomic DNA, but do not amplify hamster genomic DNA. The PCR results are converted into a data vector that is submitted to the Whitehead/MIT Radiation Mapping site (www-seq.wi.mit.edu). The data is scored and the chromosomal assignment and placement relative to known Sequence Tag Site (STS) markers on the radiation hybrid map is provided. Alternatively, the genomic sequences corresponding to nucleic acids encoding a RIPPA polypeptide are mapped by comparison to sequences in public and proprietary databases, such as the GenBank non-redundant database (ncbi.nlm.nih.gov/BLAST), Locuslink (ncbi.nlm.nih.gov:80/LocusLink/), Unigene (ncbi.nlm.nih.gov/cgi-bin/UniGene), AceView (ncbi.nlm.nih.gov/AceView), Online Mendelian Inheritance%in Man (OMIM) (ncbi.nlm.nih.gov/Omim), Gene Map Viewer (ncbi.nlm.nih.gov/genemap), and proprietary databases such as the Celera Discovery System (celera.com). These computer analyses of available genomic sequence information can provide the identification of the specific chromosomal location of genomic sequences corresponding to sequences encoding RIPPA and RIPPA-Like polypeptides, and the unique genetic mapping relationships between the RIPPA and RIPPA-Like genomic sequences and the genetic map locations of known human genetic disorders.

Diagnostics and Gene Therapy. The nucleic acids encoding RIPPA and RIPPA-Like polypeptides, and the disclosed fragments and combinations of these nucleic acids can be used by one skilled in the art using well-known techniques to analyze abnormalities associated with the genes corresponding to these polypeptides. This enables one to distinguish conditions in which this marker is rearranged or deleted. In addition, nucleic acids of the invention or a fragment thereof can be used as a positional marker to map other genes of unknown location. The DNA can be used in developing treatments for any disorder mediated (directly or indirectly) by defective, or insufficient amounts of, the genes corresponding to the nucleic acids of the invention. Disclosure herein of native nucleotide sequences permits the detection of defective genes, and the replacement thereof with normal genes. Defective genes can be detected in in vitro diagnostic assays, and by comparison of a native nucleotide sequence disclosed herein with that of a gene derived from a person suspected of harboring a defect in this gene.

Methods of Screening for Binding Partners. The RIPPA nd RIPPA-Like polypeptides of the invention each can be used as reagents in methods to screen for or identify binding partners. For example, the RIPPA or RIPPA-Like polypeptides can be attached to a solid support material and may bind to their binding partners in a manner similar to affinity chromatography. In particular embodiments, a polypeptide is attached to a solid support by conventional procedures. As one example, chromatography columns containing functional groups that will react with functional groups on amino acid side chains of polypeptides are available (Pharmacia Biotech, Inc., Piscataway, N.J.). In an alternative, a polypeptide/Fc polypeptide (as discussed above) is attached to protein A- or protein G-containing chromatography columns through interaction with the Fc moiety. The RIPPA and RIPPA-Like polypeptides also find use in identifying cells that express a RIPPA or RIPPA-Like binding partner on the cell surface. Purified RIPPA and/or RIPPA-Like polypeptides are bound to a solid phase such as a column chromatography matrix or a similar suitable substrate. For example, magnetic microspheres can be coated with the polypeptides and held in an incubation vessel through a magnetic field. Suspensions of cell mixtures containing potential binding-partner expressing cells, are contacted with the solid phase having the polypeptides thereon. Cells expressing the binding partner on the cell surface bind to the fixed polypeptides, and unbound cells are washed away. Alternatively, RIPPA and/or RIPPA-Like polypeptides can be conjugated to a detectable moiety, then incubated with cells to be tested for binding partner expression. After incubation, unbound labeled matter is removed and the presence or absence of the detectable moiety on the cells is determined. In a further alternative, mixtures of cells suspected of expressing the binding partner are incubated with biotinylated polypeptides. Incubation periods are typically at least one hour in duration to ensure sufficient binding. The resulting mixture then is passed through a column packed with avidin-coated beads, whereby the high affinity of biotin for avidin provides binding of the desired cells to the beads. Procedures for using avidin-coated beads are known (see Berenson, et al. J. Cell. Biochem., 10D:239, 1986). Washing to remove unbound material, and the release of the bound cells, are performed using conventional methods. In some instances, the above methods for screening for or identifying binding partners may also be used or modified to isolate or purify such binding partner molecules or cells expressing them.

Measuring Biological Activity. Polypeptides also find use in measuring the biological activity of RIPPA- and/or RIPPA-Like-binding polypeptides in terms of their binding affinity. The polypeptides thus can be employed by those conducting “quality assurance” studies, e.g., to monitor shelf life and stability of polypeptide under different conditions. For example, the polypeptides can be employed in a binding affinity study to measure the biological activity of a binding partner polypeptide that has been stored at different temperatures, or produced in different cell types. The polypeptides also can be used to determine whether biological activity is retained after modification of a binding partner polypeptide (e.g., chemical modification, truncation, mutation, etc.). The binding affinity of the modified polypeptide is compared to that of an unmodified binding polypeptide to detect any adverse impact of the modifications on biological activity of the binding polypeptide. The biological activity of a binding polypeptide thus can be ascertained before it is used in a research study, for example.

Carriers and Delivery Agents. The polypeptides of the invention and modified forms thereof also find use as transporters for delivering agents to cells. The polypeptides thus can be used to deliver diagnostic or therapeutic agents to such cells in in vitro or in vivo procedures. Detectable (diagnostic) and therapeutic agents that can be attached to a polypeptide include, but are not limited to, toxins, other cytotoxic agents, drugs, radionuclides, chromophores, enzymes that catalyze a colorimetric or fluorometric reaction, and the like, with the particular agent being chosen according to the intended application. Among the toxins are ricin, abrin, diphtheria toxin, Pseudomonas aeruginosa exotoxin A, ribosomal inactivating polypeptides, mycotoxins such as trichothecenes, and derivatives and fragments (e.g., single chains) thereof. Radionuclides suitable for diagnostic use include, but are not limited to, ¹²³I, ¹³¹I, ^{99m}Tc, ¹¹¹In, and ⁷⁶Br. Examples of radionuclides suitable for therapeutic use are ¹³¹I, ²¹¹At, ⁷⁷Br, ¹⁸⁶Re, ¹⁸⁸Re, ²¹²Pb, ²¹²Bi, ¹⁰⁹Pd, ⁶⁴Cu, and ⁶⁷Cu. Such agents can be attached to the polypeptide by any suitable conventional procedure. The polypeptide comprises functional groups on amino acid side chains that can be reacted with functional groups on a desired agent to form covalent bonds, for example. Alternatively, the polypeptide or agent can be derivatized to generate or attach a desired reactive functional group. The derivatization can involve attachment of one of the bifunctional coupling reagents available for attaching various molecules to polypeptides (Pierce Chemical Company, Rockford, Ill.). A number of techniques for radiolabeling polypeptides are known. Radionuclide metals can be attached to polypeptides by using a suitable bifunctional chelating agent, for example. Conjugates comprising polypeptides and a suitable diagnostic or therapeutic agent (preferably covalently linked) are thus prepared. The conjugates are administered or otherwise employed in an amount appropriate for the particular application.

Treating Diseases with RIPPA and/or RIPPA-Like Polypeptides, and Antagonists or Agonists Thereof

The RIPPA and RIPPA-Like polypeptides, fragments, variants, antagonists, agonists, antibodies, and binding partners of the invention are likely to be useful for treating medical conditions and diseases including, but not limited to, osteopenias and osteopetrosis, as well as associated conditions as described further herein. The therapeutic molecule or molecules to be used will depend on the etiology of the condition to be treated and the biological pathways involved, and variants, fragments, and binding partners of RIPPA and/or RIPPA-Like polypeptides may have effects similar to or different from RIPPA or RIPPA-Like polypeptides. For example, an antagonist of the cation transport activity of RIPPA and/or RIPPA-Like polypeptides can be selected for treatment of conditions involving cation transport activity, but a particular fragment of a given RIPPA or RIPPA-Like polypeptide may also act as an effective dominant negative antagonist of that activity. Therefore, in the following paragraphs “RIPPA polypeptides or antagonists” refers to all RIPPA or RIPPA-Like polypeptides, fragments, variants, antagonists, agonists, antibodies, and binding partners etc. of the invention, and it is understood that specific molecule or molecules can be selected from those provided as embodiments of the invention by individuals of skill in the art, according to the biological and therapeutic considerations described herein.

RIPPA and RIPPA-Like polypeptides are linked to osteoclastogenesis and/or osteoclastic bone resorption processes. Therefore, RIPPA and RIPPA-Like polypeptides are directly or indirectly implicated in diseases or conditions characterized by excessive bone resorption, generally referred to as osteopenias. As such, methods are provided for treating such disorders by administering RIPPA and RIPPA-Like polypeptides and/or antagonists of RIPPA and RIPPA-Like polypeptides, as well as antagonists to their substrates, ligands, receptors, binding partners, and or other interacting polypeptides.

Exemplary osteopenic conditions that may be treated with RIPPA and RIPPA-Like antagonists include, but are not limited to: osteoporosis, osteomyelitis, hypercalcemia, osteopenia brought on by surgery or steroid administration, prosthetic loosening, Paget's disease, osteonecrosis, bone loss due to rheumatoid arthritis, periodontal bone loss, and cancers that may metastasize to bone and induce bone breakdown.

With regards to cancer, some investigators have observed that certain cancer cells secrete a soluble form of RANKL that appears to contribute to hypercalcemia or to the establishment of malignant bone lesions (Nagai et al., Biochem Biophys Res Comm 269:532-536 (2000); and Zhang et al., 2001). Overproduction of parathyroid hormone-related protein also is believed to contribute to the hypercalcemia of cancer (see, for example, Rankin et al., Cancer (Suppl) 80(8):1564-71 (1997)). Hypercalcemia, a late complication of cancer, disrupts the body's ability to maintain a normal level of calcium, and can result in fatigue, calcium deposits in the kidneys, heart problems and neural dysfunction. Hypercalcemia occurs most frequently in patients with lung and breast cancer, and also is known to occur in patients with multiple myeloma, head and neck cancer, sarcoma, cancer of unknown primary origin, lymphoma, leukemia, melanoma, kidney cancer, and the gastrointestinal cancers, which includes esophageal, stomach, intestinal, colon and rectal cancers. The appearance of hypercalcemia has grave prognostic significance for cancer patients, with death following in one to three months for a majority of those in which it is present. Embodiments of the present invention are drawn to methods of treating hypercalcaemia by administering antagonists of RIPPA and RIPPA-Like polypeptides, as well as antagonists to their substrates, ligands, receptors, binding partners, and or other interacting polypeptides.

In one embodiment, methods described herein are used for treating patients having prostate cancer. In alternative embodiments, methods are provided for treating patients who are in the early stages of prostate cancer and who are not hypercalcemic. Such patients are in stages A, B or C of prostate cancer, as determined according to the Jewett staging system. Using this staging system, stage A is a clinically undetectable tumor confined to the prostate gland and is an incidental finding at prostatic surgery; stage B is a tumor that is confined to the prostate gland; stage C is clinically localized to the periprostatic area but extending through the prostatic capsule and may involve seminal vesicles stage D is metastatic disease. Alternatively, premetastatic prostate cancer patients may be identified by using the revised “TNM system,” which involves separate assessments of the primary tumor (T), lymph nodes (N) and metastases (M). The revised TNM system employs the same broad tumor stage (T stage) categories as the Jewett system, but includes subcategories of T stage, and PSA screening. Patients who are categorized as Stage I or stage II using this method are pre-metastatic, and are treated in accord with the present method.

Provided herein are methods of treating stage 0, I, II and III breast cancer in non-hypercalcemic patients by administering one or more RIPPA and/or RIPPA-like antagonists. For breast cancer, Stage 0 is called noninvasive carcinoma or carcinoma in situ, stages I and II are early stages in which the cancer has spread beyond the lobe or duct and invaded nearby tissue, stage III is locally advanced cancer, and stage IV is metastatic cancer.

The subject methods are useful for treating non-hypercalcemic patients with stage I and stage II renal or kidney cancer, including renal cell cancer and Wilm's tumor. For renal/kidney cancers staged in accord with NCI guidelines, stages I and II represent disease in which no cancer cells have penetrated the capsule that contains the kidney.

Provided herein are methods of treating stage 0, I, II and III lung cancer in non-hypercalcemic lung cancer patients by administering to a patient in need thereof one or more RIPPA and/or RIPPA-like antagonists. According to the currently used system for staging lung cancers, stages 0-III are non-metastastic, while stage IV is metastatic. Lung cancers include the non-small cell lung cancers, which are named for the type of cells found in the cancer and include squamous cell carcinoma (also called epidermoid carcinoma), adenocarcinoma, large cell carcinoma, adenosquamous carcinoma, and undifferentiated carcinoma. The subject methods for treating lung cancer includes treatment for the small cell lung cancers, including small cell carcinoma, mixed small cell/large cell carcinoma, combined small cell carcinoma (small cell lung cancer combined with neoplastic squamous and/or glandular components), and other neuroendocrine carcinomas of the lung, including the bronchial carcinoids, and the well-differentiated neuroendocrine carcinoma of the lung (also called malignant carcinoid, metastasizing bronchial adenoma, pleomorphic carcinoid, nonbenign carcinoid tumor, or atypical carcinoid).

Conversely, RIPPA and RIPPA-Like polypeptides may also be implicated in diseases or conditions characterized by a decrease in the rate of bone resportion, generally referred to as osteopetrosis, which is characterized by excessive bone density. For example, Infantile Malignant Osteoporosis, and the like. Alternative embodiments are drawn to methods of treating osteopetrosis by administering RIPPA and RIPPA-Like polypeptides and/or agonists of RIPPA and RIPPA-Like polypeptides, as well as agonists to their substrates, ligands, receptors, binding partners, and or other interacting polypeptides.

Compositions and methods described herein may be used in combination therapies. More specifically, methods of treating the medical conditions described herein using RIPPA and RIPPA-Like polypeptides, as well as agonists or antagonists thereto as well as to their substrates, ligands, receptors, binding partners, and or other interacting polypeptides may be used in conjunction with soluble cytokine receptors or cytokines, or other osteoclast/osteoblast regulatory molecules is also contemplated. Embodiments include, but are not limited to combination therapies with therapeutic agents targeting one or more of the following: RANK signaling through Jun NH₂-terminal kinase (JNK) and NF-κcB pathways; TNF-receptor associated factor (TRAF) adapter molecules; αvβ3 integrin receptor, such as treatment with disintegrins, such as echistatin and/or kistrin; cathepsin K; vacuolar H⁺-adenosine triphosphate, such as bafflomycin; and/or the carbonic anhydrase II (CA2) enzyme. Further examples of combination therapies include soluble forms of RANK, RANK:Fc and OPG. One or more of any of the above combination therapies may be used to treat the diseases described herein.

In treating osteopenic conditions, such as osteoporosis, bone loss may far exceed the amount that can be restored by inhibitors of resoprtion. Therefore, stimulators of bone formation, generally refered to as anabolics, may be included in treatment therapies for osteopenic conditions. For example, factors that stimulate the proliferation of osteoblasts may be used in combination therapy with RIPPA and RIPPA-Like polypeptides, as well as agonists or antagonists thereto as well as to their substrates, ligands, receptors, binding partners, and or other interacting polypeptides. Additional examples of anabolics include, but are not limited to: parathyroid hormone and secretogogues thereof; prostaglandin E and secretogogues thereof; agents that activate the promoter of bone morphogenetic protein-2 (BMP-2) gene; statins, such as inhibitors of hydroxy-methy-glutaryl-CoA (HMG-CoA), such as lovastatin and simvastatin; agents that activate the core binding protein factor-a1, a key transcription factor in osteoblast differentiation and maintenance of the differentiated state; leptin and agents that mimic the effects of leptin, and other similar agents that influence bone homeostasis; and, growth factors, such as insulin-like growth factor, transforming growth factor-beta, fibroblast growth factors, bone morphogenetic proteins, and the like.

Further embodiments are drawn to treating conditions and diseases that share cation exchange disregulation as a common feature in their etiology. For example, NHE antiporters have been implicated in a number of pathological conditions, such as chronic metabolic acidosis and alkalosis; myocardial, cerebral and renal ischaemic and reperfusion pathology; aberrant cerebral functioning including abnormal memory and cognitive functions; congenital sodium diarrhea; gastrointestinal pathologies; coronary artery diseases, such as acute responses to coronary occlusion; chronic hypertension; renal disease; diabetes and diabetes induced vascular hypertrophy; epilepsy; cancers, such as gliomas; and, gial and astrogial pathologies. Thus, antagonists or agonists of the RIPPA and RIPPA-Like polypeptides, as: well as antagonists or agonists to their substrates, ligands, receptors, binding partners, and or other interacting polypeptides described herein may be used in methods of treating patients suffering from such disorders.

The disclosed RIPPA polypeptides or antagonists, compositions and combination therapies described herein are useful in medicines for treating bacterial, viral or protozoal infections, and complications resulting therefrom. One such disease is Mycoplasma pneumonia. In addition, provided herein is the use of RIPPA polypeptides or antagonists to treat AIDS and related conditions, such as AIDS dementia complex, AIDS associated wasting, lipidistrophy due to antiretroviral therapy; and Kaposi's sarcoma. Provided herein is the use of RIPPA polypeptides or antagonists for treating protozoal diseases, including malaria and schistosomiasis. Additionally provided is the use of RIPPA polypeptides or antagonists to treat erythema nodosum leprosum; bacterial or viral meningitis; tuberculosis, including pulmonary tuberculosis; and pneumonitis secondary to a bacterial or viral infection. Provided also herein is the use of RIPPA polypeptides or antagonists to prepare medicaments for treating louse-borne relapsing fevers, such as that caused by Borrelia recurrentis. The RIPPA polypeptides or antagonists of the invention can also be used to prepare a medicament for treating conditions caused by Herpes viruses, such as herpetic stromal keratitis, corneal lesions, and virus-induced corneal disorders. In addition, RIPPA polypeptides or antagonists can be used in treating human papillomavirus infections. The RIPPA polypeptides or antagonists of the invention are used also to prepare medicaments to treat influenza.

Cardiovascular disorders are treatable with the disclosed RIPPA polypeptides or antagonists, pharmaceutical compositions or combination therapies, including aortic aneurisms; arteritis; vascular occlusion, including cerebral artery occlusion; complications of coronary by-pass surgery; ischemia/reperfusion injury; heart disease, including atherosclerotic heart disease, myocarditis, including chronic autoimmune myocarditis and viral myocarditis; heart failure, including chronic heart failure (CHF), cachexia of heart failure; myocardial infarction; restenosis after heart surgery; silent myocardial ischemia; post-implantation complications of left ventricular assist devices; Raynaud's phenomena; thrombophlebitis; vasculitis, including Kawasaki's vasculitis; giant cell arteritis, Wegener's granulomatosis; and Schoenlein-Henoch purpura.

A combination of at least one RIPPA polypeptide or antagonist and one or more other anti-angiogenesis factors may be used to treat solid tumors, thereby reducing the vascularization that nourishes the tumor tissue. Suitable anti-angiogenic factors for such combination therapies include IL-8 inhibitors, angiostatin, endostatin, kringle 5, inhibitors, of vascular endothelial growth factor (such as antibodies against vascular endothelial growth factor), angiopoietin-2 or other antagonists of angiopoietin-1, antagonists of platelet-activating factor and antagonists of basic fibroblast growth factor

In addition, the subject RIPPA polypeptides or antagonists, compositions and combination therapies are used to treat chronic pain conditions, such as chronic pelvic pain, including chronic prostatitis/pelvic pain syndrome. As a further example, RIPPA polypeptides or antagonists and the compositions and combination therapies of the invention are used to treat post-herpetic pain.

Provided also are methods for using RIPPA polypeptides or antagonists, compositions or combination therapies to treat various disorders of the endocrine system. For example, the RIPPA polypeptides or antagonists are used to treat juvenile onset diabetes (includes autoimmune and insulin-dependent types of diabetes) and also to treat maturity onset diabetes (includes non-insulin dependent and obesity-mediated diabetes). In addition, the subject compounds, compositions and combination therapies are used to treat secondary conditions associated with diabetes, such as diabetic retinopathy, kidney transplant rejection in diabetic patients, obesity-mediated insulin resistance, and renal failure, which itself may be associated with proteinurea and hypertension. Other endocrine disorders also are treatable with these compounds, compositions or combination therapies, including polycystic ovarian disease, X-linked adrenoleukodystrophy, hypothyroidism and thyroiditis, including Hashimoto's thyroiditis (i.e., autoimmune thyroiditis).

Conditions of the gastrointestinal system also are treatable with RIPPA polypeptides or antagonists, compositions or combination therapies, including coeliac disease. In addition, the compounds, compositions and combination therapies of the invention are used to treat Crohn's disease; ulcerative colitis; idiopathic gastroparesis; pancreatitis, including chronic pancreatitis and lung injury associated with acute pancreatitis; and ulcers, including gastric and duodenal ulcers.

Included also are methods for using the subject RIPPA polypeptides or antagonists, compositions or combination therapies for treating disorders of the genitourinary system, such as glomerulonephritis, including autoimmune glomerulonephritis, glomerulonephritis due to exposure to toxins or glomerulonephritis secondary to infections with haemolytic streptococci or other infectious agents. Also treatable with the compounds, compositions and combination therapies of the invention are uremic syndrome and its clinical complications (for example, renal failure, anemia, and hypertrophic cardiomyopathy), including uremic syndrome associated with exposure to environmental toxins, drugs or other causes. Further conditions treatable with the compounds, compositions and combination therapies of the invention are complications of hemodialysis; prostate conditions, including benign prostatic hypertrophy, nonbacterial prostatitis and chronic prostatitis; and complications of hemodialysis.

Also provided herein are methods for using RIPPA polypeptides or antagonists, compositions or combination therapies to treat various hematologic and oncologic disorders. For example, RIPPA polypeptides or antagonists are used to treat various forms of cancer, including acute myelogenous leukemia, Epstein-Barr virus-positive nasopharyngeal carcinoma, glioma, colon, stomach, prostate, renal cell, cervical and ovarian cancers, lung cancer (SCLC and NSCLC), including cancer-associated cachexia, fatigue, asthenia, paraneoplastic syndrome of cachexia and hypercalcemia. Additional diseases treatable with the subject RIPPA polypeptides or antagonists, compositions or combination therapies are solid tumors, including sarcoma, osteosarcoma, and carcinoma, such as adenocarcinoma (for example, breast cancer) and squamous cell carcinoma. In addition, the subject compounds, compositions or combination therapies are useful for treating leukemia, including acute myelogenous leukemia, chronic or acute lymphoblastic leukemia and hairy cell leukemia. Other malignancies with invasive metastatic potential can be treated with the subject compounds, compositions and combination therapies, including multiple myeloma. In addition, the disclosed RIPPA polypeptides or antagonists, compositions and combination therapies can be used to treat anemias and hematologic disorders, including anemia of chronic disease, aplastic anemia, including Fanconi's aplastic anemia; idiopathic thrombocytopenic purpura (ITP); myelodysplastic syndromes (including refractory anemia, refractory anemia with ringed sideroblasts, refractory anemia with excess blasts, refractory anemia with excess blasts in transformation); myelofibrosis/myeloid metaplasia; and sickle cell vasocclusive crisis.

Various lymphoproliferative disorders also are treatable with the disclosed RIPPA polypeptides or antagonists, compositions or combination therapies. These include, but are not limited to autoimmune lymphoproliferative syndrome (ALPS), chronic lymphoblastic leukemia, hairy cell leukemia, chronic lymphatic leukemia, peripheral T-cell lymphoma, small lymphocytic lymphoma, mantle cell lymphoma, follicular lymphoma, Burkitt's lymphoma, Epstein-Barr virus-positive T cell lymphoma, histiocytic lymphoma, Hodgkin's disease, diffuse aggressive lymphoma, acute lymphatic leukemias, T gamma lymphoproliferative disease, cutaneous B cell lymphoma, cutaneous T cell lymphoma (i.e., mycosis fungoides) and Sézary syndrome.

In addition, the subject RIPPA polypeptides or antagonists, compositions and combination therapies are used to treat hereditary conditions such as Gaucher's disease, Huntington's disease, linear IgA disease, and muscular dystrophy.

Other conditions treatable by the disclosed RIPPA polypeptides or antagonists, compositions and combination therapies include those resulting from injuries to the head or spinal cord, and including subdural hematoma due to trauma to the head.

The disclosed RIPPA polypeptides or antagonists, compositions and combination therapies are further used to treat conditions of the liver such as hepatitis, including acute alcoholic hepatitis, acute drug-induced or viral hepatitis, hepatitis A, B and C, sclerosing cholangitis and inflammation of the liver due to unknown causes.

In addition, the disclosed RIPPA polypeptides or antagonists, compositions and combination therapies are used to treat various disorders that involve hearing loss and that are associated with abnormal TNF(X expression. One of these is inner ear or cochlear nerve-associated hearing loss that is thought to result from an autoimmune process, i.e., autoimmune hearing loss. This condition currently is treated with steroids, methotrexate and/or cyclophosphamide, which may be administered concurrently with the RIPPA polypeptides or antagonists. Also treatable with the disclosed RIPPA polypeptides or antagonists, compositions, and combination therapies is cholesteatoma, a middle ear disorder often associated with hearing loss.

In addition, the subject invention provides RIPPA polypeptides or antagonists, compositions and combination therapies for the treatment of non-arthritic medical conditions of the bones and joints. This encompasses osteoclast disorders that lead to bone loss, such as but not limited to osteoporosis, including post-menopausal osteoporosis, periodontitis resulting in tooth loosening or loss, and prosthesis loosening after joint replacement (generally associated with an inflammatory response to wear debris). This latter condition also is called “orthopedic implant osteolysis.” Another condition treatable by administering RIPPA polypeptides or antagonists, is temporal mandibular joint dysfunction (TMJ).

A number of pulmonary disorders also can be treated with the disclosed RIPPA polypeptides or antagonists, compositions and combination therapies. One such condition is adult respiratory distress syndrome (ARDS), which is associated with elevated TNFα, and may be triggered by a variety of causes, including exposure to toxic chemicals, pancreatitis, trauma or other causes. The disclosed compounds, compositions and combination therapies of the invention also are useful for treating broncho-pulmonary dysplasia (BPD); lymphangioleiomyomatosis; and chronic fibrotic lung disease of preterm infants. In addition, the compounds, compositions and combination therapies of the invention are used to treat occupational lung diseases, including asbestosis, coal worker's pneumoconiosis, silicosis or similar conditions associated with long-term exposure to fine particles. In other aspects of the invention, the disclosed compounds, compositions and combination therapies are used to treat pulmonary disorders, including chronic obstructive pulmonary disease (COPD) associated with chronic bronchitis or emphysema; fibrotic lung diseases, such as cystic fibrosis, idiopathic pulmonary fibrosis and radiation-induced pulmonary fibrosis; pulmonary sarcoidosis; and allergies, including allergic rhinitis, contact dermatitis, atopic dermatitis and asthma.

Cystic fibrosis is an inherited condition characterized primarily by the accumulation of thick mucus, predisposing the patient to chronic lung infections and obstruction of the pancreas, which results in malabsorption of nutrients and malnutrition. RIPPA polypeptides or antagonists may be administered to treat cystic fibrosis. If desired, treatment with RIPPA polypeptides or antagonists may be administered concurrently with corticosteroids, mucus-thinning agents such as inhaled recombinant deoxyribonuclease I (such as PULMOZYME®; Genentech, Inc.) or inhaled tobramycin (TOBI®; Pathogenesis, Inc.). The RIPPA polypeptides or antagonists of the invention also may be administered concurrently with corrective gene therapy, drugs that stimulate cystic fibrosis cells to secrete chloride or other yet-to-be-discovered treatments. Sufficiency of treatment may be assessed, for example, by observing a decrease in the number of pathogenic organisms in sputum or lung lavage (such as Haemophilus influenzae, Stapholococcus aureus, and Pseudomonas aeruginosa), by monitoring the patient for weight gain, by detecting an increase in lung capacity or by any other convenient means.

The RIPPA polypeptides or antagonists of the invention, optionally combined with the cytokine IFNγ-1b (such as ACTIMMUNE®; InterMune Pharmaceuticals) may be used for treating cystic fibrosis or fibrotic lung diseases, such as idiopathic pulmonary fibrosis, radiation-induced pulmonary fibrosis and bleomycin-induced pulmonary fibrosis. In addition, this combination is useful for treating other diseases characterized by organ fibrosis, including systemic sclerosis (also called “scleroderma”), which often involves fibrosis of the liver. For treating cystic fibrosis, RIPPA polypeptides or antagonists and IFNγ-1b may be combined with PULMOZYME® or TOBI® or other treatments for cystic fibrosis.

The RIPPA polypeptides or antagonists of the invention alone or in combination with IFNγ-1b may be administered together with other treatments presently used for treating fibrotic lung disease. Such additional treatments include glucocorticoids, azathioprine, cyclophosphamide, penicillamine, colchisicine, supplemental oxygen and so forth. Patients with fibrotic lung disease, such as IPF, often present with nonproductive cough, progressive dyspnea, and show a restrictive ventilatory pattern in pulmonary function tests. Chest radiographs reveal fibrotic accumulations in the patient's lungs. When treating fibrotic lung disease in accord with the disclosed methods, sufficiency of treatment can be detected by observing a decrease in the patient's coughing (when cough is present), or by using standard lung function tests to detect improvements in total lung capacity, vital capacity, residual lung volume or by administering a arterial blood gas determination measuring desaturation under exercising conditions, and showing that the patient's lung function has improved according to one or more of these measures. In addition, patient improvement can be determined through chest radiography results showing that the progression of fibrosis in the patient's lungs has become arrested or reduced.

In addition, RIPPA polypeptides or antagonists (including soluble RIPPA polypeptides or antibodies against RIPPA polypeptides) are useful for treating organ fibrosis when administered in combination with relaxin, a hormone that down-regulates collagen production thus inhibiting fibrosis, or when given in combination with agents that block the fibrogenic activity of TGF-β. Combination therapies using RIPPA polypeptides or antagonists and recombinant human relaxin are useful, for example, for treating systemic sclerosis or fibrotic lung diseases, including cystic fibrosis, idiopathic pulmonary fibrosis, radiation-induced pulmonary fibrosis and bleomycin-induced pulmonary fibrosis.

Other embodiments provide methods for using the disclosed RIPPA polypeptides or antagonists, compositions or combination therapies to treat a variety of rheumatic disorders. These include: adult and juvenile rheumatoid arthritis; systemic lupus erythematosus; gout; osteoarthritis; polymyalgia rheumatica; seronegative spondylarthropathies, including ankylosing spondylitis; and Reiter's disease. The subject RIPPA polypeptides or antagonists, compositions and combination therapies are used also to treat psoriatic arthritis and chronic Lyme arthritis. Also treatable with these compounds, compositions and combination therapies are Still's disease and uveitis associated with rheumatoid arthritis. In addition, the compounds, compositions and combination therapies of the invention are used in treating disorders resulting in inflammation of the voluntary muscle, including dermatomyositis and polymyositis. Moreover, the compounds, compositions ant combinations disclosed herein are useful for treating sporadic inclusion body myositis, as TNFα may play a significant role in the progression of this muscle disease. In addition, the compounds, compositions and combinations disclosed herein are used to treat multicentric reticulohistiocytosis, a disease in which joint destruction and papular nodules of the face and hands are associated with excess production of proinflammatory cytokines by multinucleated giant cells.

The RIPPA polypeptides or antagonists, compositions and combination therapies of the invention may be used to inhibit hypertrophic scarring, a phenomenon believed to result in part from excessive TNFα secretion. The RIPPA polypeptides or antagonists of the invention may be administered alone or concurrently with other agents that inhibit hypertrophic scarring, such as inhibitors of TGF-α.

Cervicogenic headache is a common form of headache arising from dysfunction in the neck area, and which is associated with elevated levels of TNFα, which are believed to mediate an inflammatory condition that contributes to the patient's discomfort (Martelletti, Clin Exp Rheumatol 18(2 Suppl 19):S33-8 (March-April, 2000)). Cervicogenic headache may be treated by administering RIPPA polypeptides or antagonists as disclosed herein, thereby reducing the inflammatory response and associated headache pain.

The RIPPA polypeptides or anitagonists, compositions and combination therapies of the invention are useful for treating primary amyloidosis. In addition, the secondary amyloidosis that is characteristic of various conditions also are treatable with RIPPA polypeptides or antagonists such as RIPPA polypeptides or antagonists, and the compositions and combination therapies described herein. Such conditions include: Alzheimer's disease, secondary reactive amyloidosis; Down's syndrome; and dialysis-associated amyloidosis. Also treatable with the compounds, compositions and combination therapies of the invention are inherited periodic fever syndromes, including familial Mediterranean fever, hyperimmunoglobulin D and periodic fever syndrome and TNF-receptor associated periodic syndromes (TRAPS).

Disorders associated with transplantation also are treatable with the disclosed RIPPA polypeptides or antagonists, compositions or combination therapies, such as graft-versus-host disease, and complications resulting from solid organ transplantation, including transplantion of heart, liver, lung, skin, kidney or other organs. RIPPA polypeptides or antagonists may be administered, for example, to prevent or inhibit the development of bronchiolitis obliterans after lung transplantation.

Ocular disorders also are treatable with the disclosed RIPPA polypeptides or antagonists, compositions or combination therapies, including rhegmatogenous retinal detachment, and inflammatory eye disease, and inflammatory eye disease associated with smoking and macular degeneration.

The RIPPA polypeptides or antagonists of the invention and the disclosed compositions and combination therapies also are useful for treating disorders that affect the female reproductive system. Examples include, but are not limited to, multiple implant failure/infertility; fetal loss syndrome or IV embryo loss (spontaneous abortion); preeclamptic pregnancies or eclampsia; and endometriosis.

In addition, the disclosed RIPPA polypeptides or antagonists, compositions and combination therapies are useful for treating obesity, including treatment to bring about a decrease in leptin formation. Also, the compounds, compositions and combination therapies of the invention are used to treat sciatica, symptoms of aging, severe drug reactions (for example, Il-2 toxicity or bleomycin-induced pneumopathy and fibrosis), or to suppress the inflammatory response prior, during or after the transfusion of allogeneic red blood cells in cardiac or other surgery, or in treating a traumatic injury to a limb or joint, such as traumatic knee injury. Various other medical disorders treatable with the disclosed RIPPA polypeptides or antagonists, compositions and combination therapies include: multiple sclerosis; Behcet's syndrome; Sjogren's syndrome; autoimmune hemolytic anemia; beta thalassemia; amyotrophic lateral sclerosis (Lou Gehrig's Disease); Parkinson's disease; and tenosynovitis of unknown cause, as well as various autoimmune disorders or diseases associated with hereditary deficiencies.

The disclosed RIPPA polypeptides or antagonists, compositions and combination therapies furthermore are useful for treating acute polyneuropathy; anorexia nervosa; Bell's palsy; chronic fatigue syndrome; transmissible dementia, including Creutzfeld-Jacob disease; demyelinating neuropathy; Guillain-Barre syndrome; vertebral disc disease; Gulf war syndrome; myasthenia gravis; silent cerebral ischemia; sleep disorders, including narcolepsy and sleep apnea; chronic neuronal degeneration; and stroke, including cerebral ischemic diseases.

Disorders involving the skin or mucous membranes also are treatable using the disclosed RIPPA polypeptides or antagonists, compositions or combination therapies. Such disorders include acantholytic diseases, including Darier's disease, keratosis follicularis and pemphigus vulgaris. Also treatable with the subject RIPPA polypeptides or antagonists, compositions and combination therapies are acne; acne rosacea; alopecia areata; aphthous stomatitis; bullous pemphigoid; burns; eczema; erythema, including erythema multiforme and erythema multiforme bullosum (Stevens-Johnson syndrome); inflammatory skin disease; lichen planus; linear IgA bullous disease (chronic bullous dermatosis of childhood); loss of skin elasticity; mucosal surface ulcers; neutrophilic dermatitis (Sweet's syndrome); pityriasis rubra pilaris; psoriasis; pyoderma gangrenosum; and toxic epidermal necrolysis.

The RIPPA polypeptides or antagonists of the invention may also exhibit one or more of the following additional activities or effects: inhibiting the growth, infection or function of, or killing, infectious agents, including, without limitation, bacteria, viruses, fungi and other parasites; effecting (suppressing or enhancing) bodily characteristics, including, without limitation, height, weight, hair color, eye color, skin, fat to lean ratio or other tissue pigmentation, or organ or body part size or shape (such as, for example, breast augmentation or diminution, change in bone form or shape); effecting biorhythms or caricadic cycles or rhythms; effecting the fertility of male or female subjects; effecting the metabolism, catabolism, anabolism, processing, utilization, storage or elimination of dietary fat, lipid, polypeptide, carbohydrate, vitamins, minerals, cofactors or other nutritional factors or component(s); effecting behavioral characteristics, including, without limitation, appetite, libido, stress, cognition (including cognitive disorders), depression (including depressive disorders) and violent behaviors; providing analgesic effects or other pain reducing effects; promoting differentiation and growth of embryonic stem cells in lineages other than hematopoietic lineages; hormonal or endocrine activity; in the case of enzymes, correcting deficiencies of the enzyme and treating deficiency-related diseases; treatment of hyperproliferative disorders (such as, for example, psoriasis); immunoglobulin-like activity (such as, for example, the ability to bind antigens or complement); and the ability to act as an antigen in a vaccine composition to raise an immune response against such polypeptide or another material or entity which is cross-reactive with such polypeptide.

Administration of RIPPA and RIPPA-Like Polypeptides and Antagonists Thereof

This invention provides compounds, compositions, and methods for treating a patient, preferably a mammalian patient, and most preferably a human patient, who is suffering from a medical disorder, and in particular a disorder mediated by RIPPA and/or RIPPA-Like polypeptides. Such RIPPA-mediated disorders include conditions caused (directly or indirectly) or exacerbated by binding between RIPPA or RIPPA-Like polypeptide and a binding partner. For purposes of this disclosure, the terms “illness,” “disease,” “medical condition,” “abnormal condition” and the like are used interchangeably with the term “medical disorder.” The terms “treat”, “treating” , and “treatment” used herein includes curative, preventative (e.g., prophylactic) and palliative or ameliorative treatment. For such therapeutic uses, RIPPA and/or RIPPA-Like polypeptides and fragments, RIPPA and/or RIPPA-Like nucleic acids encoding the RIPPA and/or RIPPA-Like family polypeptides, and/or agonists or antagonists of the RIPPA and/or RIPPA-Like polypeptides such as antibodies can be administered to the patient in need through well-known means. Compositions of the present invention can contain a polypeptide in any form described herein, such as native polypeptides, variants, derivatives, oligomers, and biologically active fragments. In particular embodiments, the composition comprises a soluble polypeptide or an oligomer comprising soluble RIPPA and/or RIPPA-Like polypeptides.

Therapeutically Effective Amount. In practicing the method of treatment or use of the present invention, a therapeutically effective amount of a therapeutic agent of the present invention is administered to a patient having a condition to be treated, preferably to treat or ameliorate diseases associated with the activity of a RIPPA and/or RIPPA-Like family polypeptide. “Therapeutic agent” includes without limitation any of the RIPPA or RIPPA-Like polypeptides, fragments, and variants; nucleic acids encoding the RIPPA or RIPPA-Like family polypeptides, fragments, and variants; agonists or antagonists of the RIPPA and/or RIPPA-Like polypeptides such as antibodies; RIPPA and/or RIPPA-Like polypeptide binding partners; complexes formed from the RIPPA and/or RIPPA-Like family polypeptides, fragments, variants, and binding partners, etc. As used herein, the term “therapeutically effective amount” means the total amount of each therapeutic agent or other active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual therapeutic agent or active ingredient, administered alone, the term refers to that ingredient alone. When applied to a combination, the term refers to combined amounts of the ingredients that result in the therapeutic, effect, whether administered in combination, serially or simultaneously. As used herein, the phrase “administering a therapeutically effective amount” of a therapeutic agent means that the patient is treated with said therapeutic agent in an amount and for a time sufficient to induce an improvement, and preferably a sustained improvement, in at least one indicator that reflects the severity of the disorder. An improvement is considered “sustained” if the patient exhibits the improvement on at least two occasions separated by one or more days, or more preferably, by one or more weeks. The degree of improvement is determined based on signs or symptoms, and determinations can also employ questionnaires that are administered to the patient, such as quality-of-life questionnaires. Various indicators that reflect the extent of the patient's illness can be assessed for determining whether the amount and time of the treatment is sufficient. The baseline value for the chosen indicator or indicators is established by examination of the patient prior to administration of the first dose of the therapeutic agent. Preferably, the baseline examination is done within about 60 days of administering the first dose. If the therapeutic agent is being administered to treat acute symptoms, the first dose is administered as soon as practically possible after the injury has occurred. Improvement is induced by administering therapeutic agents such as RIPPA and/or RIPPA-Like polypeptides or antagonists until the patient manifests an improvement over baseline for the chosen indicator or indicators. In treating chronic conditions, this degree of improvement is obtained by repeatedly administering this medicament over a period of at least a month or more, e.g., for one, two, or three months or longer, or indefinitely. A period of one to six weeks, or even a single dose, often is sufficient for treating injuries or other acute conditions. Although the extent of the patient's illness after treatment may appear improved according to one or more indicators, treatment may be continued indefinitely at the same level or at a reduced dose or frequency. Once treatment has been reduced or discontinued, it later may be resumed at the original level if symptoms should reappear.

Dosing. One skilled in the pertinent art will recognize that suitable dosages will vary, depending upon such factors as the nature and severity of the disorder to be treated, the patient's body weight, age, general condition, and prior illnesses and/or treatments, and the route of administration. Preliminary doses can be determined according to animal tests, and the scaling of dosages for human administration is performed according to art-accepted practices such as standard dosing trials. For example, the therapeutically effective dose can be estimated initially from cell culture assays. The dosage will depend on the specific activity of the compound and can be readily determined by routine experimentation. A dose can be formulated in animal models to achieve a circulating plasma concentration range that includes the IC50 (i.e., the concentration of the test compound which achieves a half-maximal inhibition of symptoms) as determined in cell culture, while minimizing toxicities. Such information can be used to more accurately determine useful doses in humans. Ultimately, the attending physician will decide the amount of polypeptide of the present invention with which to treat each individual patient. Initially, the attending physician will administer low doses of polypeptide of the present invention and observe the patient's response. Larger doses of polypeptide of the present invention can be administered until the optimal therapeutic effect is obtained for the patient, and at that point the dosage is not increased further. It is contemplated that the various pharmaceutical compositions used to practice the method of the present invention should contain about 0.01 ng to about 100 mg (or about 0.1 ng to about 10 mg, or about 0.1 microgram to about 1 mg) of polypeptide of the present invention per kg body weight. In one embodiment of the invention, RIPPA polypeptides or antagonists are administered one time per week to treat the various medical disorders disclosed herein, in another embodiment is administered at least two times per week, and in another embodiment is administered at least three times per week. If injected, the effective amount of RIPPA polypeptides or antagonists per adult dose ranges from 1-20 mg/m², and preferably is about 5-12 mg/m². Alternatively, a flat dose can be administered, whose amount may range from 5-100 mg/dose. Exemplary dose ranges for a flat dose to be administered by subcutaneous injection are 5-25 mg/dose, 25-50 mg/dose and 50-100 mg/dose. In one embodiment of the invention, the various indications described below are treated by administering a preparation acceptable for injection containing RIPPA polypeptides or antagonists at 25 mg/dose, or alternatively, containing 50 mg per dose. The 25 mg or 50 mg dose can be administered repeatedly, particularly for chronic conditions. If a route of administration other than injection is used, the dose is appropriately adjusted in accord with standard medical practices. In many instances, an improvement in a patient's condition will be obtained by injecting a dose of about 25 mg of RIPPA polypeptides or antagonists one to three times per week over a period of at least three weeks, or a dose of 50 mg of RIPPA polypeptides or antagonists one or two times per week for at least three weeks, though treatment for longer periods may be necessary to induce the desired degree of improvement. For incurable chronic conditions, the regimen can be continued indefinitely, with adjustments being made to dose and frequency if such are deemed necessary by the patient's physician. The foregoing doses are examples for an adult patient who is a person who is 18 years of age or older. For pediatric patients (age 4-17), a suitable regimen involves the subcutaneous injection of 0.4 mg/kg, up to a maximum dose of 25 mg of RIPPA polypeptides or antagonists, administered by subcutaneous injection one or more times per week. If an antibody against a RIPPA polypeptide is used as the RIPPA polypeptide antagonist, a preferred dose range is 0.1 to 20 mg/kg, and more preferably is 1-10 mg/kg. Another preferred dose range for an anti-RIPPA polypeptide antibody is 0.75 to 7.5 mg/kg of body weight. Humanized antibodies are preferred, that is, antibodies in which only the antigen-binding portion of the antibody molecule is derived from a non-human source. Such antibodies can be injected or administered intravenously.

Formulations. Compositions comprising an effective amount of a RIPPA polypeptide of the present invention (from whatever source derived, including without limitation from recombinant and non-recombinant sources), in combination with other components such as a physiologically acceptable diluent, carrier, or excipient, are provided herein. The term “pharmaceutically acceptable” means a non-toxic material that does not interfere with the effectiveness of the biological activity of the active ingredient(s). Formulations suitable for administration include aqueous and non-aqueous sterile injection solutions which can contain anti-oxidants, buffers, bacteriostats and solutes which render the formulation isotonic with the blood of the recipient; and aqueous and non-aqueous sterile suspensions which can include suspending agents or thickening agents. The polypeptides can be formulated according to known methods used to prepare pharmaceutically useful compositions. They can be combined in admixture, either as the sole active material or with other known active materials suitable for a given indication, with pharmaceutically acceptable diluents (e.g., saline, Tris-HCl, acetate, and phosphate buffered solutions), preservatives (e.g., thimerosal, benzyl alcohol, parabens), emulsifiers, solubilizers, adjuvants and/or carriers. Suitable formulations for pharmaceutical compositions include those described in Remington's Pharmaceutical Sciences, 16th ed. 1980, Mack Publishing Company, Easton, Pa. In addition, such compositions can be complexed with polyethylene glycol (PEG), metal ions, or incorporated into polymeric compounds such as polyacetic acid, polyglycolic acid, hydrogels, dextran, etc., or incorporated into liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,235,871; U.S. Pat. No. 4,501,728; U.S. Pat. No. 4,837,028; and U.S. Pat. No. 4,737,323. Such compositions will influence the physical state, solubility, stability, rate of in vivo release, and rate of in vivo clearance, and are thus chosen according to the intended application, so that the characteristics of the carrier will depend on the selected route of administration. In one preferred embodiment of the invention, sustained-release forms of RIPPA or RIPPA-Like polypeptides are used. Sustained-release forms suitable for use in the disclosed methods include, but are not limited to, RIPPA and/or RIPPA-Like polypeptides that are encapsulated in a slowly-dissolving biocompatible polymer (such as the alginate microparticles described in U.S. Pat. No. 6,036,978), admixed with such a polymer (including topically applied hydrogels), and or encased in a biocompatible semi-permeable implant.

Combinations of Therapeutic Compounds. A RIPPA or RIPPA-Like polypeptide of the present invention may be active in multimers (e.g., heterodimers or homodimers) or complexes with itself or other polypeptides. As a result, pharmaceutical compositions of the invention may comprise a polypeptide of the invention in such multimeric or complexed form. The pharmaceutical composition of the invention may be in the form of a complex of the polypeptide(s) of present invention along with polypeptide or peptide antigens. The invention further includes the administration of RIPPA and/or RIPPA-Like polypeptides or antagonists concurrently with one or more other drugs that are administered to the same patient in combination with the RIPPA and/or RIPPA-Like polypeptides or antagonists, each drug being administered according to a regimen suitable for that medicament. “Concurrent administration” encompasses simultaneous or sequential treatment with the components of the combination, as well as regimens in which the drugs are alternated, or wherein one component is administered long-term and the other(s) are administered intermittently. Components can be administered in the same or in separate compositions, and by the same or different routes of administration. Examples of components that can be administered concurrently with the pharmaceutical compositions of the invention are: cytokines, lymphokines, or other hematopoietic factors such as M-CSF, GM-CSF, TNF, IL-1, IL-2, IL-3, IL4, IL-5, IL-6, IL-7, IL-8, IL-9, IL-10, IL-11, IL-12, IL-13, IL-14, IL-15,IL-17, IL-18, IFN, TNF0, TNF1, TNF2, G-CSF, Meg-CSF, thrombopoietin, stem cell factor, and erythropoietin, or inhibitors or antagonists of any of these factors. The pharmaceutical composition can further contain other agents which either enhance the activity of the polypeptide or compliment its activity or use in treatment. Such additional factors and/or agents may be included in the pharmaceutical composition to produce a synergistic effect with polypeptide of the invention, or to minimize side effects. Conversely, a RIPPA polypeptide or antagonist of the present invention may be included in formulations of the particular cytokine, lymphokine, other hematopoietic factor, thrombolytic or anti-thrombotic factor, or anti-inflammatory agent to minimize side effects of the cytokine, lymphokine, other hematopoietic factor, thrombolytic or anti-thrombotic factor, or anti-inflammatory agent. Additional examples of drugs to be administered concurrently include but are not limited to antivirals, antibiotics, analgesics, corticosteroids, antagonists of inflammatory cytokines, non-steroidal anti-inflammatories, pentoxifylline, thalidomide, and disease-modifying antirheumatic drugs (DMARDs) such as azathioprine, cyclophosphamide, cyclosporine, hydroxychloroquine sulfate, methotrexate, leflunomide, minocycline, penicillamine, sulfasalazine and gold compounds such as oral gold, gold sodium thiomalate, and aurothioglucose. Additionally, RIPPA polypeptides or antagonists can be combined with a second RIPPA polypeptide/antagonist, including an antibody against a RIPPA and/or RIPPA-Like polypeptide, or a RIPPA or RIPPA-Like polypeptide-derived peptide that acts as a competitive inhibitor of a native RIPPA and/or RIPPA-Like polypeptide.

In practicing the subject therapeutic methods, the RIPPA and/or RIPPA-Like antagonist is administered to a non-hypercalcemic cancer patient whose cancer has not metastasized to bone in an amount and at a frequency of administration that is effective to reach one or more of the following endpoints: a reduction in tumor burden; a stabilization of tumor burden; a slowing of the growth rate of the malignant cells; an increase in the length of time the patient remains disease free; and an increase in the length of time during which the cancer does not progress. In yet another aspect of the invention, the RIPPA and/or RIPPA-Like antagonist is administered in an amount and at a frequency that is effective to reduce the amount of a surrogate marker that is associated with a particular type of cancer. Examples of such surrogate markers are serum HER2/neu in breast cancer and serum PSA for prostate cancer. The RIPPA and/or RIPPA-Like antagonist may be administered to patients prior to or immediately following surgical removal of a solid tumor, or at any time post-surgery.

The duration of treatment will vary, but typically repeated doses will be administered over at least a period of two weeks or longer, or may be adminstered indefinitely. Several rounds of treatment may be given, alternating with periods of no treatment. If discontinued, treatment may be resumed if a relapse of the cancer should occur.

Treatment of cancer with a RIPPA and/or RIPPA-Like antagonist may be administered concurrently with other treatments, and usually will be administered concurrently with chemotherapy or radiation treatment. In one example, the RIPPA and/or RIPPA-Like antagonist is given concurrently with an agent that is effective against a variety of tumor types, such as Apo2 ligand/TRAIL or an anti-angiogenic agent such as an antibody against VEGF or an antibody against the EGF receptor. The RIPPA and/or RIPPA-Like antagonist treatment also may be combined with other treatments that target specific kinds of cancer, such as for example, monoclonal antibodies targeted to tumor-specific antigens, or with other treatments used for particular kinds of cancer. For example, breast cancer may treated with a RIPPA and/or RIPPA-Like antagonist administered concurrently with chemotherapy, hormone treatment, tamoxifen, raloxifene or agents that target HER2, such as an anti-HER2 antibody such as HERCEPTIN® (Genentech, Inc.), or any combination thereof. In another example, chronic lymphocytic leukemia or non-Hodgkin's lymphoma is treated with a combination of a RIPPA and/or RIPPA-Like antagonist and the anti-CD20 monoclonal antibody RITUXIN® (Genentech, Inc.). The invention also contemplates the concurrent administration of RIPPA and/or RIPPA-Like antagonists with various soluble cytokine receptors or cytokines or other drugs used for chemotherapy of cancer. “Concurrent administration” encompasses simultaneous or sequential treatment with the components of the combination, as well as regimens in which the drugs are alternated, or wherein one component is administered long-term and the other(s) are administered intermittently. Such other drugs include, for example, bisphosphonates used to restore bone loss in cancer patients, or the use of more than one RANK antagonist administered concurrently. Examples of other drugs to be administered concurrently include but are not limited to antivirals, antibiotics, analgesics, corticosteroids, antagonists of inflammatory cytokines, DMARDs, various systemic chemotherapy regimens and non-steroidal anti-inflammatories, such as, for example, COX I or COX II inhibitors.

Routes of Administration. Any efficacious route of administration can be used to therapeutically administer RIPPA polypeptides or antagonists thereof, including those compositions comprising nucleic acids. Parenteral administration includes injection, for example, via intra-articular, intravenous, intramuscular, intralesional, intraperitoneal or subcutaneous routes by bolus injection or by continuous infusion., and also includes localized administration, e.g., at a site of disease or injury. Other suitable means of administration include sustained release from implants; aerosol inhalation and/or insufflation; eyedrops; vaginal or rectal suppositories; buccal preparations; oral preparations, including pills, syrups, lozenges, ice creams, or chewing gum; and topical preparations such as lotions, gels, sprays, ointments or other suitable techniques. Alternatively, polypeptideaceous RIPPA polypeptides or antagonists may be administered by implanting cultured cells that express the polypeptide, for example, by implanting cells that express RIPPA polypeptides or antagonists. Cells may also be cultured ex vivo in the presence of polypeptides of the present invention in order to modulate cell proliferation or to produce a desired effect on or activity in such cells. Treated cells can then be introduced in vivo for therapeutic purposes. The polypeptide of the instant invention may also be administered by the method of protein transduction. In this method, the RIPPA or RIPPA-Like polypeptide is covalently linked to a protein-transduction domain (PTD) such as, but not limited to, TAT, Antp, or VP22 (Schwarze et al., 2000, Cell Biology 10: 290-295). The PTD-linked peptides can then be transduced into cells by adding the peptides to tissue-culture media containing the cells (Schwarze et al., 1999, Science 285: 1569; Lindgren et al., 2000, TiPS 21: 99; Derossi et al., 1998, Cell Biology 8: 84; WO 00/34308; WO 99/29721; and WO 99/10376). In another embodiment, the patient's own cells are induced to produce RIPPA polypeptides or antagonists by transfection in vivo or ex vivo with a DNA that encodes RIPPA olypeptides or antagonists. This DNA can be introduced into the patient's cells, for example, by injecting naked DNA or liposome-encapsulated DNA that encodes RIPPA polypeptides or antagonists, or by other means of transfection. Nucleic acids of the invention can also be administered to patients by other known methods for introduction of nucleic acid into a cell or organism (including, without limitation, in the form of viral vectors or naked DNA). When RIPPA polypeptides or antagonists are administered in combination with one or more other biologically active compounds, these can be administered by the same or by different routes, and can be administered simultaneously, separately or sequentially.

Oral Administration. When a therapeutically effective amount of polypeptide of the present invention is administered orally, polypeptide of the present invention will be in the form of a tablet, capsule, powder, solution or elixir. When administered in tablet form, the pharmaceutical composition of the invention can additionally contain a solid carrier such as a gelatin or an adjuvant. The tablet, capsule, and powder contain from about 5 to 95% polypeptide of the present invention, and preferably from about 25 to 90% polypeptide of the present invention. When administered in liquid form, a liquid carrier such as water, petroleum, oils of animal or plant origin such as peanut oil, mineral oil, soybean oil, or sesame oil, or synthetic oils can be added. The liquid form of the pharmaceutical composition can further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of polypeptide of the present invention, and preferably from about 1 to 50% polypeptide of the present invention.

Intravenous Administration. When a therapeutically effective amount of polypeptide of the present invention is administered by intravenous, cutaneous or subcutaneous injection, polypeptide of the present invention will be in the form of a pyrogen-free, parenterally acceptable aqueous solution. The preparation of such parenterally acceptable polypeptide solutions, having due regard to pH, isotonicity, stability, and the like, is within the skill in the art. A preferred pharmaceutical composition for intravenous, cutaneous, or subcutaneous injection should contain, in addition to polypeptide of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention can also contain stabilizers, preservatives, buffers, antioxidants, or other additives known to those of skill in the art. The duration of intravenous therapy using the pharmaceutical composition of the present invention will vary, depending on the severity of the disease being treated and the condition and potential idiosyncratic response of each individual patient. It is contemplated that the duration of each application of the polypeptide of the present invention will be in the range of 12 to 24 hours of continuous intravenous administration. Ultimately the attending physician will decide on the appropriate duration of intravenous therapy using the pharmaceutical composition of the present invention.

Bone and Tissue Administration. For compositions of the present invention which are useful for bone, cartilage, tendon or ligament disorders, the therapeutic method includes administering the composition topically, systematically, or locally as an implant or device. When administered, the therapeutic composition for use in this invention is, of course, in a pyrogen-free, physiologically acceptable form. Further, the composition can desirably be encapsulated or injected in a viscous form for delivery to the site of bone, cartilage or tissue damage. Topical administration may be suitable for wound healing and tissue repair. Therapeutically useful agents other than a polypeptide of the invention which may also optionally be included in the composition as described above, can alternatively or additionally, be administered simultaneously or sequentially with the composition in the methods of the invention. Preferably for bone and/or cartilage formation, the composition would include a matrix capable of delivering the polypeptide-containing composition to the site of bone and/or cartilage damage, providing a structure for the developing bone and cartilage and optimally capable of being resorbed into the body. Such matrices can be formed of materials presently in use for other implanted medical applications. The choice of matrix material is based on biocompatibility, biodegradability, mechanical properties, cosmetic appearance and interface properties. The particular application of the compositions will define the appropriate formulation. Potential matrices for the compositions can be biodegradable and chemically defined calcium sulfate, tricalciumphosphate, hydroxyapatite, polylactic acid, polyglycolic acid and polyanhydrides. Other potential materials are biodegradable and biologically well-defined, such as bone or dermal collagen. Further matrices are comprised of pure polypeptides or extracellular matrix components. Other potential matrices are nonbiodegradable and chemically defined, such as sintered hydroxapatite, bioglass, aluminates, or other ceramics Matrices can be comprised of combinations of any of the above mentioned types of material, such as polylactic acid and hydroxyapatite or collagen and tricalciumphosphate. The bioceramics can be altered in composition, such as in calcium-aluminate-phosphate and processing to alter pore size, particle size, particle shape, and biodegradability. Presently preferred is a 50:50 (mole weight) copolymer of lactic acid and glycolic acid in the form of porous particles having diameters ranging from 150 to 800 microns. In some applications, it will be useful to utilize a sequestering agent, such as carboxymethyl cellulose or autologous blood clot, to prevent the polypeptide compositions from disassociating from the matrix. A preferred family of sequestering agents is cellulosic materials such as alkylcelluloses (including hydroxyalkylcelluloses), including methylcellulose, ethylcellulose, hydroxyethylcellulose, hydroxypropylcellulose, hydroxypropyl-methylcellulose, and carboxymethyl-cellulose, the most preferred being cationic salts of carboxymethylcellulose (CMC). Other preferred sequestering agents include hyaluronic acid, sodium alginate, poly(ethylene glycol), polyoxyethylene oxide, carboxyvinyl polymer and poly(vinyl alcohol). The amount of sequestering agent useful herein is 0.5-20 wt %, preferably 1-10 wt % based on total formulation weight, which represents the amount necessary to prevent desorbtion of the polypeptide from the polymer matrix and to provide appropriate handling of the composition, yet not so much that the progenitor cells are prevented from infiltrating the matrix, thereby providing the polypeptide the opportunity to assist the osteogenic activity of the progenitor cells. In further compositions, polypeptides of the invention may be combined with other agents beneficial to the treatment of the bone and/or cartilage defect, wound, or tissue in question. These agents include various growth factors; such as epidermal growth factor (EGF), platelet derived growth factor (PDGF), transforming growth factors (TGF-alpha and TGF-beta), and insulin-like growth factor (IGF). The therapeutic compositions are also presently valuable for veterinary applications. Particularly domestic animals and thoroughbred horses, in addition to humans, are desired patients for such treatment with polypeptides of the present invention. The dosage regimen of a polypeptide-containing pharmaceutical composition to be used in tissue regeneration will be determined by the attending physician considering various factors which modify the action of the polypeptides, e.g., amount of tissue weight desired to be formed, the site of damage, the condition of the damaged tissue, the size of a wound, type of damaged tissue (e.g., bone), the patient's age, sex, and diet, the severity of any infection, time of administration and other clinical factors. The dosage can vary with the type of matrix used in the reconstitution and with inclusion of other polypeptides in the pharmaceutical composition. For example, the addition of other known growth factors, such as IGF I (insulin like growth factor I), to the final composition, may also effect the dosage. Progress can be monitored by periodic assessment of tissue/bone growth and/or repair, for example, X-rays, histomorphometric determinations and tetracycline labeling.

Veterinary Uses. In addition to human patients, RIPPA polypeptides and antagonists are useful in the treatment of disease conditions in non-human animals, such as pets (dogs, cats, birds, primates, etc.), domestic farm animals (horses cattle, sheep, pigs, birds, etc.), or any animal that suffers from a condition mediated by RIPPA and/or RIPPA-Like polypeptides. In such instances, an appropriate dose can be determined according to the animal's body weight. For example, a dose of 0.2-1 mg/kg may be used. Alternatively, the dose is determined according to the animal's surface area, an exemplary dose ranging from 0.1-20 mg/m², or more preferably, from 5-12 mg/m². For small animals, such as dogs or cats, a suitable dose is 0.4 mg/kg. In a preferred embodiment, RIPPA polypeptides or antagonists (preferably constructed from genes derived from the same species as the patient), is administered by injection or other suitable route one or more times per week until the animal's condition is improved, or it can be administered indefinitely.

Manufacture of Medicaments. The present invention also relates to the use of RIPPA and/or RIPPA-Like polypeptides, fragments, and variants; nucleic acids encoding the RIPPA or RIPPA-Like family polypeptides, fragments, and variants; agonists or antagonists of the RIPPA and/or RIPPA-Like polypeptides such as antibodies; RIPPA and/or RIPPA-Like polypeptide binding partners; complexes formed from the RIPPA and/or RIPPA-Like family polypeptides, fragments, variants, and binding partners, etc, in the manufacture of a medicament for the prevention or therapeutic treatment of each medical disorder disclosed herein.

### EXAMPLES

The following examples are intended to illustrate particular embodiments and not to limit the scope of the invention.

### Example 1

### Identification of Human and Murine RIPPA, New Na⁺/H⁺ Antiporter Polypeptides

RIPPA (RANKL-Induced Proton Pump Analog) nucleic acid and polypeptide sequences were identified following the discovery that expression of a particular mouse EST (GenBank Accession Number AV251613) was very strongly increased (expression up to at least 100-fold higher than unstimulated expression) in the murine RAW macrophage cell line within 24 hours of exposure to RANKL (RANK Ligand). The sequence of this mouse EST was used to identify additional overlapping murine EST and genomic sequences, and their human counterparts. The full-length human RIPPA cDNA sequence is presented as SEQ ID NO: 1 and encodes human RIPPA polypeptides having the amino acid sequences shown in SEQ ID NO:2 through SEQ ID NO:5. Nucleotides 84 through 1694 of SEQ ID NO:1 encode SEQ ID NO:2, with nucleotides 1695 through 1697 of SEQ ID NO:1 corresponding to a stop codon. The mouse RIPPA cDNA sequence is shown as SEQ ID NO:6; nucleotides 1 through 1641 encode the murine RIPPA polypeptide with the amino acid sequence of SEQ ID NO:7. Nucleotides 1642 through 1644 of SEQ ID NO:6 correspond to a stop codon.

The human RIPPA coding sequences were compared with publicly available preliminary human genomic DNA sequences, and the following human chromosome 4 contig was identified as containing RIPPA coding sequences: GenBank accession number AC097485.1. The human genomic chromosme 4q24 region corresponding to these contigs also includes the genetic loci for Wolfram Syndrome gene 2 (WFS2) and RAP1 (GTPase-GDP Dissociation Stimulator 1 or GDS1); thus nucleic acid probes designed from human RIPPA coding sequences can be used to identify this chromosomal region and to more precisely map such genetic loci. The approximate positions of the exons containing RIPPA coding sequence in the AC097485.1 contig are shown in the table below, along with their locations relative to SEQ ID NO:1; note that the 5′ and 3′ untranslated regions may extend further along the contig sequence beyond those portions that correspond to SEQ ID NO:1, as indicated by the parentheses around the AC097485.1 endpoints in the table. The human RIPPA-Like polypeptide (GeneSeq AAA16638 nucleotide sequence and GeneSeq AAY94918 polypeptide sequence) has also been mapped to a location on human chromosome 4 adjacent to that of human RIPPA; human RIPPA like coding sequences are present on contigs with accession numbers GenBank AP001860.2, AC080124.3, and AC083826.7. This suggests that the evolutionary ancestors of human RIPPA and human RIPPA-Like arose from a gene duplication event. The murine RIPPA coding sequence (SEQ ID NO:6) was compared with public mouse genome sequences, and nucleotides 27024 through 27113 of Mus musculus genomic clone RP23-453I14 (GenBank AC104874.1) was found to correspond to nucleotides 1 through 90 of SEQ ID NO:6; presumably the murine genomic sequence 5′ to nucleotide 27024 in clone RP23-453I14 corresponds to the 5′ UTR and promoter region for transcription of the mouse RIPPA gene. The murine coding sequence was also compared with the assembly of Mus musculus genomic sequences available from Celera Genomics as release 12; mouse exons corresponding to the cDNA sequence were identified in reverse orientation in GA_X5J8B7W6U34_{—}197, a 16,904,947-bp portion of the Mus chromosome 3 from position 115,515,146 to 132,420,093. The murine RIPPA exons correspond to the 3′ portion of the human RIPPA exon 2 through the 5′ portion of exon 13, and are located in the genomic assembly (with position 115,515,146 represented as 1) and in SEQ ID NO:6 as shown in the table below; note that the 5′ and 3′ untranlated regions likely extend along the assembly sequence beyond those portions that correspond to SEQ ID NO:6, as indicated by the parentheses around the assembly endpoints in the table.

Corresponding positions of RIPPA gene exons in human contig AC097485.1 and in cDNA sequences:

| | Nucleotide Position in: | | | |
|---|---|---|---|---|
| Exon | AC097485.1 | SEQ ID NO: 1 | Celera Mus Assembly | SEQ ID NO: 6 |
| 1 | (59949)-59989 | 1-41 | n/a | n/a |
| 2 | 68581-68712 | 42-173 | (42773)-42684 | 1-90 |
| 3 | 69666-69846 | 174-354 | 41597-41417 | 91-271 |
| 4 | 78221-78391 | 355-525 | 37991-37821 | 272-442 |
| 5 | 85810-85952 | 526-668 | 36248-36106 | 443-585 |
| 6 | 87172-87299 | 669-796 | 35368-35241 | 586-713 |
| 7 | 88966-89141 | 797-972 | 33886-33711 | 714-889 |
| 8 | 91196-91302 | 973-1079 | 30988-30882 | 890-996 |
| 9 | 92747-92896 | 1080-1229 | 30212-30063 | 997-1146 |
| 10 | 104384-104492 | 1230-1338 | 29398-29290 | 1147-1255 |
| 11 | 107309-10745 | 1339-1475 | 27565-27429 | 1256-1392 |
| 12 | 109601-109824 | 1476-1699 | 23935-23715 | 1393-1613 |
| 13 | 115803-(115871) | 1700-1768 | 18017-(17987) | 1614-1644 |

Several splice variations of human RIPPA polypeptide sequences have been identified by sequencing multiple RIPPA cDNA sequences and are included within the scope of the invention. The amino acid sequences of the splice variants that have been detected are shown in SEQ ID NO:3 through SEQ ID NO:5. In splice variant RIPPA ‘A’ (SEQ ID NO:3), the 171-nt exon 4 is not present in the cDNA, producing a protein that lacks amino acids 91 through 147 of SEQ ID NO:2 and is 480 amino acids in length rather than 537 amino acids. In splice variant RIPPA ‘B’ (SEQ ID NO:4), the 128-nt exon 6 is not present in the cDNA, and a splice acceptor site within exon 7 (between nucleotides 854 and 855 of SEQ ID NO:1) is used, removing the 58 5′ nucleotides of exon 7 and preserving the reading frame, producing a protein that lacks amino acids 196 through 257 of SEQ ID NO:2 and is 475 amino acids in length rather than 537 amino acids. Splice variant RIPPA ‘C’ (SEQ ID NO:5) combines both the variations of variants A and B, so that amino acids 91 through 147 and amino acids 196 through 257 of SEQ ID NO:2 are missing from SEQ ID NO:5, producing a protein that is 418 amino acids in length rather than 537 amino acids. The close correlation between the exon sizes and positions of the human and murine coding sequences, particularly including exons 3 through 11, raises the strong possibility that splice variants of murine RIPPA exist which correspond to the human RIPPA splice variants described above; such splice variants of murine RIPPA polypeptides are encompassed within the scope of the invention.

Additional variations of RIPPA polypeptides are provided as naturally occurring genomic variants of the RIPPA sequences disclosed herein; such variations may be incorporated into a RIPPA polypeptide or nucleic acid individually or in any combination, or in combination with alternative splice variation as described above. As one example, amino acid 260 of SEQ ID NO:3 likely represents an allelic variation, where the change from the Gly residue (at the corresponding position 317 of SEQ ID NO:2) to an Arg residue in SEQ ID NO:3 could be caused by a single change from ‘G’ to ‘A’ at position 1032 of SEQ ID NO:1. This variation and other potential allelic variations, as shown in published partial amino acid or nucleic acid sequences, are listed in the table below:

| Amino Acid | Position in SEQ ID | Nucleotide | Position in SEQ ID |
|---|---|---|---|
| Change | NO: 2 | Change | NO: 1 |
| Glu -> Asp | 11 | A -> T | 116 |
| Ser -> Cys | 16 | C -> G | 130 |
| Val -> Leu | 34 | G -> C | 183 |
| Thr -> Ser | 46 | A -> T | 219 |
| Ser -> Cys | 49 | A -> T | 228 |
| Pro -> Ser | 111 | C -> T | 414 |
| Phe -> Leu | 122 | C -> G | 449 |
| Phe -> Ile | 154 | T -> A | 543 |
| Arg -> Thr | 157 | G -> C | 553 |
| Lys -> Met | 169 | A -> T | 589 |
| Lys -> Arg | 171 | A -> G | 595 |
| Ser -> Tyr | 174 | C -> A | 604 |
| Ser -> Ala | 182 | T -> G | 627 |
| Gly -> Val | 191 | G -> T | 655 |
| Asp -> Glu | 193 | T -> G | 662 |
| Lys -> Glu | 198 | A -> G | 675 |
| Asp -> Tyr | 278 | G -> T | 915 |
| Gly -> Arg | 317 | G -> A | 1032 |
| Ala -> Ser | 411 | G -> T | 1314 |

The amino acid sequences of human and murine RIPPA (SEQ ID NOs 2 and 7) were compared with the amino acid sequences of these other Na+/H+ antiporter family members—Drosophila melanogaster GH12682p (GenBank accession number AAL13583; SEQ ID NO:8) and Methanothermobacter thermautotrophicus Na+/H+ antiporter (GenBank accession number NP_{—}275902; SEQ ID NO:9)—using the GCG “pretty” multiple sequence alignment program, with amino acid similarity scoring matrix=blosum62, gap creation penalty=8, and gap extension penalty=2. An alignment of these sequences is shown in Table 1, and includes consensus residues that are identical among at least three of the amino acid sequences in the alignment. The capitalized residues in the alignment are those which match the consensus residues.

Amino acid substitutions and other alterations (deletions, insertions, etc.) to RIPPA amino acid sequences (e.g. SEQ ID NOs 2 through 5 and 7) are predicted to be more likely to alter or disrupt RIPPA polypeptide activities if they result in changes to the capitalized residues of the amino acid sequences as shown in Table 1, and particularly if those changes do not substitute an amino acid of similar structure (such as substitution of any one of the aliphatic residues—Ala, Gly, Leu, Ile, or Val—for another aliphatic residue), or a residue present in other Na+/H+ antiporter polypeptides at that conserved position. Conversely, if a change is made to a RIPPA amino acid sequence resulting in substitution of the residue at that position in the alignment from one of the other Table 1 Na+/H+ antiporter polypeptide sequences, it is less likely that such an alteration will affect the function of the altered RIPPA polypeptide. For example, the consensus residue at position 128 in Table 1 is leucine, and one of the Na+/H+ antiporter polypeptides (SEQ ID NO:9) has a phenylalanine at that position. Substitution of phenylalanine or the chemically similar tryptophan or tyrosine for leucine at that position is less likely to alter the function of the polypeptide than substitution of a charged resiude such as lysine, arginine, etc. Embodiments of the invention include RIPPA polypeptides and fragments of RIPPA polypeptides, comprising altered amino acid sequences. Altered RIPPA polypeptide sequences share at least 30%, or at least 40%, or at least 50%, or at least 55%, or at least 60%, or at least 65%, or at least 70%, or at least 75%, or at least 80%, or at least 85%, or at least 90%, or at least 95%, or at least 97.5%, or at least 99%, or at least 99.5% amino acid identity with one or more of the Na+/H+ antiporter amino acid sequences shown in Table 1.

TABLE 1
Alignment of RIPPA amino acid sequences with those of other Na+/H+
antiporter polypeptides

Protein, SEQ
ID NO:

| | 1 | 50 |
|---|---|---|
| m RIPPA, 7 | mededktaec ghskpptgit heapphhelq eervMslrGt drsep....t | |
| h RIPPA, 2 | mgdedkrity edsepstgmn ytpsmhgeaq eetvMklkGi danep....t | |
| Drosoph, 8 | ˜˜˜˜˜˜˜˜˜˜ ˜˜˜˜˜˜˜˜˜˜ ˜˜˜˜˜˜˜˜˜˜ ˜˜˜˜MghnGg gssngganyv | |
| Consensus | ---------- ---------- ---------- ----M---G- ---------- | |
| | 51 | 100 |
| m RIPPA, 7 | EgSnlltsge KkpqdsPt.. .EpnglQslr rflaCPprgc laRvItngtm | |
| h RIPPA, 2 | EgSillksse KklqetPt.. .EanhvQrlr qmlaCPphgl ldRvItnvti | |
| Drosoph, 8 | EeSwmynfcl KcrgeePsas wEppfwQkif pyplCPtfrt vsRlIsiili | |
| Consensus | 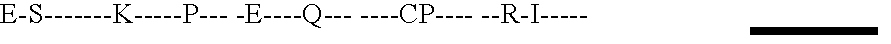 | |
| | 101 | 150 |
| m RIPPA, 7 | vVLlWamvws vtGpeclPGG nLFGiiiLfy csItGGkLfG Li..kfpTLp | |
| h RIPPA, 2 | iVLlWavvws itGseclPGG nLFGiiiLfy caIiGGkLlG Li..klpTLp | |
| Drosoph, 8 | gVLiWvtafv iiGdsaaPGG qLFGlvvLtv aanfGGyLi. .......sLt | |
| Methano, 9 | ˜˜˜˜˜˜˜˜˜˜ ˜˜˜˜˜˜˜˜˜˜ ˜˜meyvafsv avIi...LlG LlfnrvfTra | |
| Consensus | 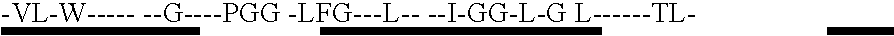 | |
| | 151 | 200 |
| m RIPPA, 7 | pLPpLLGMLL aGfLlrNipv indsvrIqhK wSssLRsIAL svILvRAGLG | |
| h RIPPA, 2 | pLPsLLGMLL aGfLirNipv indnvqIkhK wSssLRsIAL sIILvRAGLG | |
| Drosoph, 8 | tLPrLiGMLL vGiLfqNvgw adidgdfs.K vtahLRkfAL tIILtRAGLe | |
| Methano, 9 | rLPgiLGMLi lGiLigphgl nlisksImd. iSpdLRvIAL iIILlRAGfG | |
| Consensus | 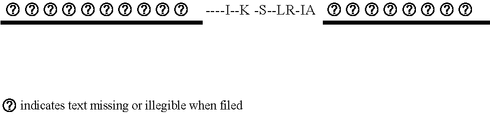 | |
| | 201 | 250 |
| m RIPPA, 7 | ldskALrKlk gvcVrLamgP ciVEAcasAi lSHfLmGLPW gWgFILGFVv | |
| h RIPPA, 2 | ldskALkKlk gvcVrLsmgP ciVEActsAl laHyLLGLPW qWgFILGFVl | |
| Drosoph, 8 | mepeAfkKvy ktilkLgiiP wcVEAvvmAv mSHfLLdLPW iWaFlLGsii | |
| Methano, 9 | inlesLrKvg mtaVkmsfiP dvfEglavmv aSHyiLGLPi ieagILGFVi | |
| Consensus | 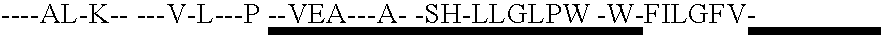 | |
| | 251 | 300 |
| m RIPPA, 7 | gAVSPAVVVP sMLlLqegGY GVgKGIPTLl mAagSfDDiL aITgFntcLG | |
| h RIPPA, 2 | gAVSPAVVVP sMLlLqggGY GVeKGvPTLl mAagSfDDiL | |
| | aITgFntcLG | |
| Drosoph, 8 | aAVSPAVVVP clfrLrtkGY GVaKGIPTLv vAvagvDDaL svaiFgiist | |
| Methano, 9 | aAVSPAViVP qMLsfierrm GtaKGIPTii ltgaSvDDvv sITlFsvfLG | |
| Consensus | 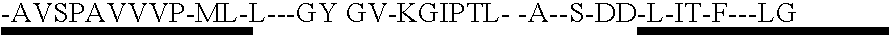 | |
| | 301 | 350 |
| m RTPPA, 7 | vaFStg.stv fnifrgiLev viGvaaGsfL GffiqYFPsR dqdnlVwkRa | |
| h RIPPA, 2 | iaFStg.stv fnvlrgvLev viGvatGsvL GffiqYFPsR dqdklVckRT | |
| Drosoph, 8 | vmFSdk.gla yqiaqapvci lgGlgfGvvw GslariFPek gdayvVplRT | |
| Methano, 9 | iyqgqqvnll veaigvpLsi ItGitgGlaL GlalvYlfrR feirnt.ekT | |
| Consensus | 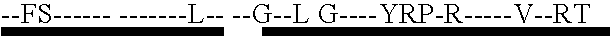 | |
| | 351 | 400 |
| m RIPPA, 7 | fLvLGfavLA vfsSvyfsFp GsGgLctLVm AFlaGm.... .rWtdkkseV | |
| h RIPPA, 2 | fLvLGlsvLA vfsSvhfgFp GsGgLctLVm AFlaGm.... .gWtsekaeV | |
| Drosoph, 8 | lLlftgglmA iygSeelgFe GaGpLavvfs AFvsnlfwck dgWdvednpV | |
| Methano, 9 | liiLGaaiLl knmgdlis.. tylpiaaLVg vmvvGlv.il ermpetglkl | |
| Consensus | 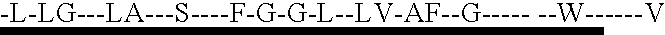 | |
| | 401 | 450 |
| m RIPPA, 7 | ekviavtWdv FqPLLFGLiG AeVsIvsLra eTVgLcvAtl siavliRILT | |
| H RIPPA, 2 | ekiiavaWdi FqPLLFGLiG AeVsIasLrp eTVgLcvAtv giavliRILT | |
| Drosoph, 8 | stafeifWmi FePiLFGLtG AtikIreLds hTVsigaAci ftgailRILT | |
| Methano, 9 | sekfnkvWif aeiLLFvLvG AqVdvr.Lif hvglLglAvi taglmvRsig | |
| Consensus | 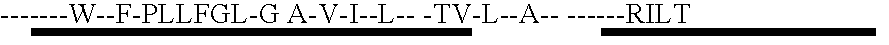 | |
| | 451 | 500 |
| m RIPPA, 7 | TfLmvcfagf NiKEKiFisf AWlPKATVQA AIGsVALdta rshGEkqled | |
| h RIPPA, 2 | TfLmvcfagf NlKEKiFisf AWlPKATVQA AIGsVALdta rshGEkqled | |
| Drosoph, 8 | Tagiafgdrl NtKEKfFvgl sWmaKATVQA AlGpVALkhl gddateeern | |
| Methano, 9 | vlLslkgtdl NlKEKvFcis AyiPKATVQA AIGaipLaag vaaGE..... | |
| Consensus | 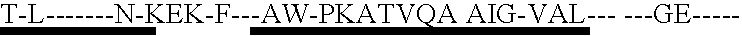 | |
| | 501 | 550 |
| m RIPPA, 7 | ygmdVLTvAf LaILiTAPiG SLlIgllGpR vLqKsehrt. eeevqGetsa | |
| h RIPPA, 2 | ygmdVLTvAf LsILiTAPiG SLlIgllGpR lLqKvehqnk deevqGetsv | |
| Drosoph, 8 | waniVqTicv fsIvlTAPIG aimIsvtGtk lLtKtk...q pqdlsGwrrs | |
| Methano, 9 | ...tiLamAv LaILfTAPIG SLavkwtGeR fLevergn˜˜ ˜˜˜˜˜˜˜˜˜˜ | |
| Consensus | 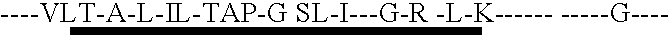 | |
| | 551 | 597 |

| | |
|---|---|
| m RIPPA, 7 | hiqrkpedsi tea˜˜˜˜˜˜˜ ˜˜˜˜˜˜˜˜˜˜ ˜˜˜˜˜˜˜˜˜˜ ˜˜˜˜˜˜˜ |
| h RIPPA, 2 | qv˜˜˜˜˜˜˜˜ ˜˜˜˜˜˜˜˜˜˜ ˜˜˜˜˜˜˜˜˜˜ ˜˜˜˜˜˜˜˜˜˜ ˜˜˜˜˜˜˜ |
| Drosoph, 8 | hrpsirdisi ideeeeredp eipedkethd ntlnnahips isytynt |
| Consensus | ---------- ---------- ---------- ---------- ------- |

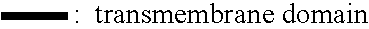

When analyzed using Hidden Markov Model predictions of potential transmembrane (TM) domains (HMMTM analysis), the form of human RIPPA that is 537 amino acids long has 13 potential TM domains, the approximate positions of which are illustrated graphically in the table above, and by amino acid location in the table below. Interestingly, the N-terminus of the 537-aa human RIPPA is predicted by HMMTM to be extracellular. Since the splice forms of human RIPPA polypeptide have the same N-terminal amino acid sequence as the RIPPA polypeptide of SEQ ID NO:2, they are shown in the table below as also having extracellular N-termini. However, when the splice forms A, B, and C of human RIPPA are anlayzed using HMMTM, the entire N-terminal region (amino acids I through 117) of RIPPA splice forms A and C (SEQ ID NOs 3 and 5) is predicted to have an intracellular location; in this context it is interesting to note that the Asn residue at position 90 of the human RIPPA polypeptides (SEQ ID NOs 2 through 5) and at position 90 of murine RIPPA (SEQ ID NO:7) is a predicted site for N-glycosylation.

TABLE 2
Location of Predicted Exterior, Transmembrane, and
Interior Domains within RIPPA Splice Forms

| Predicted | RIPPA (SEQ ID | ‘A’ (SEQ ID NO: 3) | ‘B’ (SEQ ID NO: 4) | ‘C’ (SEQ ID NO: 5) |
|---|---|---|---|---|
| Location: | NO: 2) amino acids: | amino acids: | amino acids: | amino acids: |
| N-term EXT | 1-83,^{‡} 86 | 1-83, 86 | 1-83, 86 | 1-83, 86 |
| N-term TM | 84, 87-105, 106 | 84, 87-90 . . . | 84, 87-105, 106 | 84, 87-90 . . . |
| INTERIOR | 106, 107-112 | | 106, 107-112 | |
| TM 1 | 113-135 | | 113-135 | |
| EXTERIOR | 136-138 | | 136-138 | |
| TM 2 | 139-161 | . . . 91-104 | 139-161 | . . . 91-104 |
| INTERIOR | 162-172, 173 | 105-117 | 162-172, 173 | 105-117 |
| TM 3 | 173, 174-191, 192 | 118-133, 135 | 173, 174-191, 192 | 117, 118-134 |
| EXTERIOR | 192, 193-206 | 134, 136-149 | 192, 193-195 . . . | 135-138 . . . |
| TM 4 | 207-228, 229 | 150-171 | | |
| INTERIOR | 229, 230-233 | 172-175, 176 | | |
| TM 5 | 234-256 | 176, 177-198, 199 | | |
| EXTERIOR | 257-279 | 199, 200-222 | . . . 196-217 | . . . 139-160 |
| TM 6 | 280-302 | 223-245 | 218-240 | 161-183 |
| INTERIOR | 303-305 | 246-249 | 241-243 | 184-186 |
| TM 7 | 306-328 | 250-271 | 244-266 | 187-209 |
| EXTERIOR | 329-340, 341 | 272-282, 283 | 267-278, 279 | 210-221 |
| TM 8 | 341, 342-374, 376 | 283, 284-317, 319 | 279, 280-312, 314 | 222-255, 256 |
| INTERIOR | 375, 377-387, 388 | 318, 320-331 | 313, 315-325, 326 | 256, 257-269 |
| TM 9 | 388, 389-411 | 332-354 | 326, 327-349 | 270-292 |
| EXTERIOR | 412-417, 420 | 355-360, 362 | 350-355, 358 | 293-298, 300 |
| TM 10 | 418, 421-440, 445 | 361, 363-383, 390 | 356, 359-378, 383 | 299, 301-321, 327 |
| INTERIOR | 441, 446-451, 452 | 384, 391-393, 396 | 379, 384-389, 390 | 322, 328-332, 334 |
| TM 11 | 452, 453-470 | 394, 397-413, 415 | 390, 391-408 | 333, 335-351, 353 |
| EXTERIOR | 471-489 | 414, 416-432 | 409-427 | 352, 354-370 |
| TM 12 | 490-512, 513 | 433-455, 456 | 428-450, 451 | 371-393, 394 |
| C-term INT | 513, 514-537 | 456, 457-480 | 451, 452-475 | 394, 395-418 |

^{‡}Amino acid positions separated by a comma represent a range of possible amino acid positions for the boundary of a specified region within the RIPPA polypeptide.

### Example 2

### Monoclonal Antibodies That Bind Polypeptides of the Invention

This example illustrates a method for preparing monoclonal antibodies that bind RIPPA and/or RIPPA-Like polypeptides. Other conventional techniques may be used, such as those described in U.S. Pat. No. 4,411,993. Suitable immunogens that may be employed in generating such antibodies include, but are not limited to, purified RIPPA or RIPPA-Like polypeptide, an immunogenic fragment thereof, and cells expressing high levels of RIPPA or RIPPA-Like polypeptide or an immunogenic fragment thereof. DNA encoding a RIPPA or a RIPPA-Like polypeptide can also be used as an immunogen, for example, as reviewed by Pardoll and Beckerleg in Immunity 3: 165, 1995.

Rodents (BALB/c mice or Lewis rats, for example) are immunized with RIPPA or RIPPA-Like polypeptide immunogen emulsified in an adjuvant (such as complete or incomplete Freund's adjuvant, alum, or another adjuvant, such as Ribi adjuvant R700 (Ribi, Hamilton, Mont.)), and injected in amounts ranging from 10-100 micrograms subcutaneously or intraperitoneally. DNA may be given intradermally (Raz et al., 1994, Proc. Natl. Acad. Sci. USA 91: 9519) or intamuscularly (Wang et al., 1993, Proc. Natl. Acad. Sci. USA 90: 4156); saline has been found to be a suitable diluent for DNA-based antigens. Ten days to three weeks days later, the immunized animals are boosted with additional immunogen and periodically boosted thereafter on a weekly, biweekly or every third week immunization schedule.

Serum samples are periodically taken by retro-orbital bleeding or tail-tip excision to test for anti-RIPPA or anti-RIPPA-Like polypeptide antibodies by dot-blot assay, ELISA (enzyme-linked immunosorbent assay), immunoprecipitation, or other suitable assays, such as FACS analysis of inhibition of binding of RIPPA or RIPPA-Like polypeptide to a RIPPA and/or RIPPA-Like polypeptide binding partner. Following detection of an appropriate antibody titer, positive animals are provided one last intravenous injection of RIPPA or RIPPA-Like polypeptide in saline. Three to four days later, the animals are sacrificed, and spleen cells are harvested and fused to a murine myeloma cell line, e.g., NS1 or preferably P3X63Ag8.653 (ATCC CRL-1580). These cell fusions generate hybridoma cells, which are plated in multiple microtiter plates in a HAT (hypoxanthine, aminopterin and thymidine) selective medium to inhibit proliferation of non-fused cells, myeloma hybrids, and spleen cell hybrids.

The hybridoma cells may be screened by ELISA for reactivity against purified RIPPA or RIPPA-Like polypeptide by adaptations of the techniques disclosed in Engvall et al., (Immunochem. 8: 871, 1971) and in U.S. Pat. No. 4,703,004. A preferred screening technique is the antibody capture technique described in Beckmann et al., (J. Immunol. 144: 4212, 1990). Positive hybridoma cells can be injected intraperitoneally into syngeneic rodents to produce ascites containing high concentrations (for example, greater than 1 milligram per milliliter) of anti-RIPPA or anti-RIPPA-Like polypeptide monoclonal antibodies. Alternatively, hybridoma cells can be grown in vitro in flasks or roller bottles by various techniques. Monoclonal antibodies can be purified by ammonium sulfate precipitation, followed by gel exclusion chromatography. Alternatively, affinity chromatography based upon binding of antibody to protein A or protein G can also be used, as can affinity chromatography based upon binding to RIPPA polypeptide.

### Example 3

### Antisense Inhibition of RIPPA and RIPPA-Like Nucleic Acid Expression

In accordance with the present invention, a series of oligonucleotides are designed to target different regions of the human RIPPA mRNA molecule, using the nucleotide sequence of SEQ ID NO:1 as the basis for the design of the oligonucleotides. The oligonucleotides are selected to be approximately 10, 12, 15, 18, or 20 nucleotide residues in length, and to have a predicted hybridization temperature that is at least 37 degrees C. Preferably, the oligonucleotides are selected so that some will hybridize toward the 5′ region of the mRNA molecule, others will hybridize to the coding region, and still others will hybridize to the 3′ region of the mRNA molecule. Methods such as those of Gray and Clark (U.S. Pat. Nos. 5,856,103 and 6,183,966) can be used to select oligonucleotides that form the most stable hybrid structures with target sequences, as such oligonucleotides are desirable for use as antisense inhibitors.

The oligonucleotides may be oligodeoxynucleotides, with phosphorothioate backbones (internucleoside linkages) throughout, or may have a variety of different types of internucleoside linkages. Generally, methods for the preparation, purification, and use of a variety of chemically modified oligonucleotides are described in U.S. Pat. No. 5,948,680. As specific examples, the following types of nucleoside phosphoramidites may be used in oligonucleotide synthesis: deoxy and 2′-alkoxy amidites; 2′-fluoro amidites such as 2′-fluorodeoxyadenosine amidites, 2′-fluorodeoxyguanosine, 2′-fluorouridine, and 2′-fluorodeoxycytidine; 2′-O-(2-methoxyethyl)-modified amidites such as 2,2′-anhydro[1-(beta-D-arabino-furanosyl)-5-methyluridine], 2′-O-methoxyethyl-5-methyluridine, 2′-O-methoxyethyl-5′-O-dimethoxytrityl-5-methyluridine, 3′-O-acetyl-2′-O-methoxy-ethyl-5′-O-dimethoxytrityl-5-methyluridine, 3′-O-acetyl-2′-O-methoxyethyl-5′-O-dimethoxytrityl-5-methyl-4-triazoleuridine, 2′-O-methoxyethyl-5′-O-dimethoxytrityl-5-methylcytidine, N4-benzoyl-2′-O-methoxyethyl-5′-O-dimethoxytrityl-5-methylcytidine, and N4-benzoyl-2′-O-methoxyethyl-5′-O-di-methoxytrityl-5-methylcytidine-3′-amidite; 2′-O-(aminooxyethyl) nucleoside amidites and 2′-O-(dimethylaminooxyethyl) nucleoside amidites such as 2′-(dimethylaminooxyethoxy) nucleoside amidites, 5′-O-tert-butyldiphenylsilyl-O²-2′-anhydro-5-methyluridine, 5′-O-tert-butyl-diphenylsilyl-2′-O-(2-hydroxyethyl)-5-methyluridine, 2′-O-([2-phthalimidoxy)ethyl]-5′-t-butyldiphenyl-silyl-5-methyluridine, 5′-O-tert-butyldiphenylsilyl-2′-O-[(2-formadoximnooxy)ethyl]-5-methyluridine, 5′-O-tert-butyldiphenylsilyl-2′-O-[N,N-dimethylaminooxyethyl]-5-methyluridine, 2′-O-(dimethylaminooxy-ethyl)-5-methyluridine, 5′-O-DMT-2′-O-(dimethylaminooxyethyl)-5-methyluridine, and 5′-O-DMT-2′-O-(2-N,N-dimethylaminooxyethyl)-5-methyluridine-3′-[(2-cyanoethyl)-N,N-diisopropylphosphor-amidite]; and 2′-(aminooxyethoxy) nucleoside amidites such as N2-isobutyryl-6-O-diphenyl-carbamoyl-2′-O-(2-ethylacetyl)-5′-O-(4,4′-dimethoxytrityl)guanosine-3′-(2-cyanoethyl)-N,N-diiso-propylphosphoramidite].

Modified oligonucleosides may also be used in oligonucleotide synthesis, for example methylenemethylimino-linked oligonucleosides, also called MMI-linked oligonucleosides; methylene-dimethylhydrazo-linked oligonucleosides, also called MDH-linked oligonucleosides; methylene-carbonylamino-linked oligonucleosides, also called amide-3-linked oligonucleosides; and methylene-aminocarbonyl-linked oligonucleosides, also called amide-4-linked oligonucleosides, as well as mixed backbone compounds having, for instance, alternating MMI and P═O or P═S linkages, which are prepared as described in U.S. Pat. Nos. 5,378,825, 5,386,023, 5,489,677, 5,602,240 and 5,610,289. Formacetal- and thioformacetal-linked oligonucleosides may also be used and are prepared as described in U.S. Pat. Nos. 5,264,562 and 5,264,564; and ethylene oxide linked oligonucleosides may also be used and are prepared as described in U.S. Pat. No. 5,223,618. Peptide nucleic acids (PNAs) may be used as in the same manner as the oligonucleotides described above, and are prepared in accordance with any of the various procedures referred to in Peptide Nucleic Acids (PNA): Synthesis, Properties and Potential Applications, Bioorganic & Medicinal Chemistry, 1996, 4, 5-23; and U.S. Pat. Nos. 5,539,082, 5,700,922, and 5,719,262.

Chimeric oligonucleotides, oligonucleosides, or mixed oligonucleotides/oligonucleosides of the invention can be of several different types. These include a first type wherein the “gap” segment of linked nucleosides is positioned between 5′ and 3′ “wing” segments of linked nucleosides and a second “open end” type wherein the “gap” segment is located at either the 3′ or the 5′ terminus of the oligomeric compound. Oligonucleotides of the first type are also known in the art as “gapmers” or gapped oligonucleotides. Oligonucleotides of the second type are also known in the art as “hemimers” or “wingmers”. Some examples of different types of chimeric oligonucleotides are: [2′-O-Me]-[2′-deoxy]-[2′-O-Me] chimeric phosphorothioate oligonucleotides, [2′-O-(2-methoxyethyl)]-[2′-deoxy]-[2′-O-(methoxyethyl)] chimeric phosphorothioate oligonucleotides, and [2′-O-(2-methoxy-ethyl)phosphodiester]-[2′-deoxy phosphoro-thioate]-[2′-O-(2-methoxyethyl)phosphodiester] chimeric oligonucleotides, all of which may be prepared according to U.S. Pat. No. 5,948,680. In one preferred embodiment, chimeric oligonucleotides (“gapmers”) 18 nucleotides in length are utilized, composed of a central “gap” region consisting of ten 2′-deoxynucleotides, which is flanked on both sides (5′ and 3′ directions) by four-nucleotide “wings”. The wings are composed of 2′-methoxyethyl (2′-MOE) nucleotides. The internucleoside (backbone) linkages are phosphorothioate (P═S) throughout the oligonucleotide. Cytidine residues in the 2′-MOE wings are 5-methylcytidines. Other chimeric oligonucleotides, chimeric oligonucleosides, and mixed chimeric oligonucleo-tides/oligonucleosides are synthesized according to U.S. Pat. No. 5,623,065.

Oligonucleotides are preferably synthesized via solid phase P(III) phosphorarmidite chemistry on an automated synthesizer capable of assembling 96 sequences simultaneously in a standard 96 well format. The concentration of oligonucleotide in each well is assessed by dilution of samples and UV absorption spectroscopy. The full-length integrity of the individual products is evaluated by capillary electrophoresis, and base and backbone composition is confirmed by mass analysis of the compounds utilizing electrospray-mass spectroscopy.

The effect of antisense compounds on target nucleic acid expression can be tested in any of a variety of cell types provided that the target nucleic acid is present at measurable levels. This can be routinely determined using, for example, PCR or Northern blot analysis. Cells are routinely maintained for up to 10 passages as recommended by the supplier. When cells reached 80% to 90% confluency, they are treated with oligonucleotide. For cells grown in 96-well plates, wells are washed once with 200 microliters OPTI-MEM-1 reduced-serum medium (Gibco BRL) and then treated with 130 microliters of OPTI-MEM-1 containing 3.75 g/mL LIPOFECTIN (Gibco BRL) and the desired oligonucleotide at a final concentration of 150 nM. After 4 hours of treatment, the medium is replaced with fresh medium. Cells are harvested 16 hours after oligonucleotide treatment. Preferably, the effect of several different oligonucleotides should be tested simultaneously, where the oligonucleotides hybridize to different portions of the target nucleic acid molecules, in order to identify the oligonucleotides producing the greatest degree of inhibition of expression of the target nucleic acid.

Antisense modulation of RIPPA nucleic acid expression can be assayed in a variety of ways known in the art. For example, RIPPA mRNA levels can be quantitated by, e.g., Northern blot analysis, competitive polymerase chain reaction (PCR), or real-time PCR (RT-PCR). Real-time quantitative PCR is presently preferred. RNA analysis can be performed on total cellular RNA or poly(A)+ mRNA. Methods of RNA isolation and Northern blot analysis are taught in, for example, Ausubel, F. M. et al., Current Protocols in Molecular Biology, Volume 1, pp. 4.1.1-4.2.9 and 4.5.1-4.5.3, John Wiley & Sons, Inc., 1996. Real-time quantitative (PCR) can be conveniently accomplished using the commercially available ABI PRISM 7700 Sequence Detection System, available from PE-Applied Biosystems, Foster City, Calif. and used according to manufacturer's instructions. This fluorescence detection system allows high-throughput quantitation of PCR products. As opposed to standard PCR, in which amplification products are quantitated after the PCR is completed, products in real-time quantitative PCR are quantitated as they accumulate. This is accomplished by including in the PCR reaction an oligonucleotide probe that anneals specifically between the forward and reverse PCR primers, and contains two fluorescent dyes. A reporter dye (e.g., JOE or FAM, obtained from either Operon Technologies Inc., Alameda, Calif. or PE-Applied Biosystems, Foster City, Calif.) is attached to the 5′ end of the probe and a quencher dye (e.g., TAMRA, obtained from either Operon Technologies Inc., Alameda, Calif. or PE-Applied Biosystems, Foster City, Calif.) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular (six-second) intervals by laser optics built into the ABI PRISM 7700 Sequence Detection System. In each assay, a series of parallel reactions containing serial dilutions of mRNA from untreated control samples generates a standard curve that is used to quantitate the percent inhibition after antisense oligonucleotide treatment of test samples. Other methods of quantitative PCR analysis are also known in the art. RIPPA protein levels can be quantitated in a variety of ways well known in the art, such as imnmunoprecipitation, Western blot analysis (immunoblotting), ELISA, or fluorescence-activated cell sorting (FACS). Antibodies directed to RIPPA polypeptides can be prepared via conventional antibody generation methods such as those described herein. Immunoprecipitation methods, Western blot (immunoblot) analysis, and enzyme-linked immunosorbent assays (ELISA) are standard in the art (see, for example, Ausubel, F. M. et al., Current Protocols in Molecular Biology, Volume 2, pp. 10.16.1-10.16.11, 10.8.1-10.8.21, and 11.2.1-11.2.22, John Wiley & Sons, Inc., 1991).

### Example 4

### RIPPA Is Upregulated in Macrophage Cell Line Upon Stimulation with RANK-L

The macrophage cell line RAW 264.7 was cultured under standard conditions (ATCC No. TIB-71; American Type Culture Collection, P.O. Box 1549, Manassas, Va. 20108: see, J Immunol, 1977; 119:950; Cell, 1978; 15:261). For example, Dulbecco's modified Eagle's medium with 4 mM L-glutamine adjusted to contain 1.5 g/L sodium bicarbonate and 4.5 g/L glucose, 90%; fetal bovine serum, 10% Temperature: 37C. RAW 264.7 cells were seeded in 6 well plates at approximately 5×10⁴ /well. Three assay conditions were tested: media only control, muRANKL at 100 ng/ml and TNFα at 20 ng/ml. Time points were taken at approximately 24 and 72 hours post addition of muRANKL and TNFα. Cells were lysed and total RNA isolated using standard techniques, such as those described in the RNeasy® kit provided by Qiagen, Inc., Valencia, Calif. Real time quantitative reverse transcriptase (RT)-PCR was performed under standard conditions, such as those recommended in the GenAmp 5700® system (Applied Biosystems, Foster City, Calif.). SYBR® Green PCR Master Mix was used in the PCR reactions, which detects any double-stranded DNA generated during PCR without the use of the TaqMan® probe.

The following muRIPPA and muHPRT-specific oligos were used in the RT-PCR assay:

| | |
|---|---|
| SEQ ID NO:10: | |
| muRIPPA(+) | 5′ GGT TGG CCT TTG TGT TGC A 3′ |
| SEQ ID NO:11: | |
| muRIPPA(−) | 5′ AAG CCA GCG AAA CAC ACC AT 3′ |
| SEQ ID NO:12: | |
| muHPRT(+) | 5′ GTC CCA GCG TCG TGA TTA GC 3′ |
| SEQ ID NO:13: | |
| muHPRT(−) | 5′ TTC CAA ATC CTC GGC ATA ATG 3′ |

Relative units of RIPPA DNA were quantified and normalized against a standard housekeeping gene HPRT (hypoxanthine guanine phosphorybosyl transferase).

FIG. 1 represents a RT-PCR-based assay performed on cDNA extracted from the mouse macrophage cell line RAW 264.7 post stimulation with muRANKL or TNFα. These data show that expression of RIPPA is strongly upregulated in the osteoclast precursor cell line after stimulation with RANKL but comparatively little upregulation in response to TNFα. These studies show that RIPPA is upregulated in response to RANKL, which is known to induce differentiation of monocytes/macrophages into mature osteoclasts. Therefore, RIPPA and RIPPA-Like polypeptides are involved in osteoclastogenesis and/or osteoclastic bone resorption processes.

### Example 5

### RIPPA Is Upregulated in Primary Monocyte Cultures Upon Stimulation with M-CSF/RANK-L or M-CSF/TNFα

Primary monocyte cultures were created from mouse bone marrow cultures using techniques well established in the art. After lysing red blood cells, the isolated bone marrow cells are cultured in the presence of M-CSF (macrophage-colony stimulating factor) at 40 ng/ml to drive the cell population towards a monocyte/macrophage lineage. Cells were seeded in 6 well plates at approximately 5×10⁴ per well.

Three assay conditions were tested: M-CSF control, M-CSF in combination with muRANKL at 100 ng/ml and M-CSF in combination with TNFα at 20 ng/ml. M-CSF was used at 40 ng/ml for all cultures. The monocytes were cultured in the presence of M-CSF, muRANKL, TNFα for 5 days. Cells were lysed and total RNA isolated using standard techniques, such as those described in the RNeasy® kit provided by Qiagen, Inc., Valencia, Calif. Real time quantitative reverse transcriptase (RT)-PCR was performed under standard conditions, such as those recommended in the GenAmp 5700® system (Applied Biosystems, Foster City, Calif.). SYBR® Green PCR Master Mix was used in the PCR reactions, which detects any double-stranded DNA generated during PCR without the use of the TaqMan® probe. The same oligos described in Example 4 (i.e., SEQ ID NO:10-13) were used in these studies. Relative units of RIPPA DNA were quantified and normalized against a standard housekeeping gene HPRT (hypoxanthine guanine phosphorybosyl transferase).

FIG. 2 illustrates the results from a RT-PCR-based assay performed on cDNA extracted from primary monocyte cultures post stimulation with M-CSF (Macrophage Colony Stimulating Factor) and muRANKL or TNFα. Real-time PCR analysis of RIPPA cDNA levels in bone marrow-drived primary monocyte cultures showed surprising upregualtion of RIPPA in response to M-CSF/RANKL, as well as upregulation of RIPPA in response to M-CSF/TNFα, but not to M-CSF alone These studies further confirm that RIPPA is upregulated in response to RANKL, which is known to induce differentiation of monocytes/macrophages into mature osteoclasts. Therefore, RIPPA and RIPPA-Like polypeptides are involved in osteoclastogenesis and/or osteoclastic bone resorption processes.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

Sequences Presented in the Sequence Listing

| SEQ ID NO | Type | Description |
|---|---|---|
| SEQ ID NO: 1 | Nucleotide | Human RIPPA cDNA sequence |
| SEQ ID NO: 2 | Amino acid | Human RIPPA amino acid sequence |
| SEQ ID NO: 3 | Amino acid | Human RIPPA amino acid sequence - |
| | | splice form ‘A’ |
| SEQ ID NO: 4 | Amino acid | Human RIPPA amino acid sequence - |
| | | splice form ‘B’ |
| SEQ ID NO: 5 | Amino acid | Human RIPPA amino acid sequence - |
| | | splice form ‘C’ |
| SEQ ID NO: 6 | Nucleotide | Mus musculus RIPPA cDNA |
| | | sequence |
| SEQ ID NO: 7 | Amino acid | Mus musculus RIPPA amino acid |
| | | sequence |
| SEQ ID NO: 8 | Amino acid | Drosophila melanogaster GH12682p |
| | | (GenBank AAL13583) |
| SEQ ID NO: 9 | Amino acid | Methanothermobacter |
| | | thermautotrophicus |
| | | Na+/H+-exchanging protein; |
| | | Na+/H+ antiporter (GenBank |
| | | NP_275902) |
| SEQ ID NO: 10 | Artificial seq. | Oligo #48525 for muRIPPA(+) |
| SEQ ID NO: 11 | Artificial seq. | Oligo #48526 for muRIPPA(−) |
| SEQ ID NO: 12 | Artificial seq. | Oligo #40343 for muHPRT(+) |
| SEQ ID NO: 13 | Artificial seq. | Oligo #40344 for muHPRT(−) |

What is claimed is:

1. An isolated polypeptide comprising an amino acid sequence selected from the group consisting of:
(a) the amino acid sequence of SEQ ID NO:2, SEQ ID NO:3, SEQ ID NO:4, or SEQ ID NO:5;
(b) amino acids 113 through 512 of SEQ ID NO:2, amino acids 87 through 455 of SEQ ID NO:3, amino acids 87 through 450 of SEQ ID NO:4, or amino acids 87 through 394 of SEQ ID NO:5;
(c) a fragment of the amino acid sequences of (a) comprising amino acids 514 through 537 of SEQ ID NO:2, wherein a polypeptide consisting of said fragment has cation exchange activity;
(d) amino acids 514 through 537 of SEQ ID NO:2 and an amino acid sequence selected from the group consisting of amino acids 1 through 83; 87 through 105; 107 through 112; 113 through 135; 136 through 138; 139 through 161; 162 through 172; 174 through 191; 193 through 206; 207 through 228; 230 through 233; 234 through 256; 257 through 279; 280 through 302; 303 through 305; 306 through 328; 329 through 340; 342 through 374; 377 through 387; 389 through 411; 412 through 417; 421 through 440; 446 through 451; 453 through 470; or 471 through 489 of SEQ ID NO:2;
(e) amino acids 421 through 440 of SEQ ID NO:2;
(f) an amino acid sequence comprising at least 20 amino acids and sharing amino acid identity with the amino acid sequence of (e), wherein the percent amino acid identity is selected from the group consisting of: at least 85%, at least 90%, at least 95%, at least 97.5%, at least 99%, and at least 99.5%;
(g) an amino acid sequence comprising at least 20 amino acids and sharing amino acid identity with the amino acid sequences of (e), wherein the percent amino acid identity is selected from the group consisting of: at least 85%, at least 90%, at least 95%, at least 97.5%, at least 99%, and at least 99.5%, and wherein a polypeptide comprising said amino acid sequence binds to an antibody that also binds to a polypeptide comprising an amino acid sequence of any of (a)-(d); and
(h) an amino acid sequence of (d)-(g), wherein a polypeptide consisting of said amino acid sequence has cation exchange activity.

2. An isolated polypeptide comprising an amino acid sequence selected from the group consisting of:
(a) the amino acid sequence of SEQ ID NO:7;
(b) amino acids 113 through 512 of SEQ ID NO:7;
(c) a fragment of the amino acid sequence of (a) comprising amino acids 514 through 547 of SEQ ID NO:7, wherein a polypeptide consisting of said fragment has cation exchange activity;
(d) amino acids 514 through 547 of SEQ ID NO:7 and an amino acid sequence selected from the group consisting of amino acids 1 through 83; 87 through 105; 107 through 112; 113 through 135; 136 through 138; 139 through 161; 162 through 172; 174 through 191; 193 through 206; 207 through 228; 230 through 233; 234 through 256; 257 through 279; 280 through 302; 303 through 305; 306 through 328; 329 through 340; 342 through 374; 377 through 387; 389 through 411; 412 through 417; 421 through 440; 446 through 451; 453 through 470; or 471 through 489 of SEQ ID NO:7;
(e) amino acids 421 through 440 of SEQ ID NO:7;
(f) an amino acid sequence comprising at least 20 amino acids and sharing amino acid identity with the amino acid sequence of (e), wherein the percent amino acid identity is selected from the group consisting of: at least 85%, at least 90%, at least 95%, at least 97.5%, at least 99%, and at least 99.5%;
(g) an amino acid sequence comprising at least 20 amino acids and sharing amino acid identity with the amino acid sequences of (e), wherein the percent amino acid identity is selected from the group consisting of: at least 85%, at least 90%, at least 95%, at least 97.5%, at least 99%, and at least 99.5%, and wherein a polypeptide comprising said amino acid sequence binds to an antibody that also binds to a polypeptide comprising an amino acid sequence of any of (a)-(d); and
(h) an amino acid sequence of (d)-(g), wherein a polypeptide consisting of said amino acid sequence has cation exchange activity.

3. The polypeptide of claim 1, wherein the polypeptide has cation exchange activity.

4. The polypeptide of claim 2, wherein the polypeptide has cation exchange activity.

5. An isolated nucleic acid encoding a polypeptide of claim 1.

6. An isolated nucleic acid encoding a polypeptide of claim 2.

7. The nucleic acid of claim 5, comprising a nucleotide sequence selected from the group consisting of:
(a) SEQ ID NO:1;
(b) nucleotides 84 through 1694 of SEQ ID NO:1; and
(c) allelic variants of (a)-(b).

8. The nucleic acid of claim 6, comprising a nucleotide sequence selected from the group consisting of:
(a) SEQ ID NO:6; and
(b) allelic variants of (a).

9. The nucleic acid of claim 5, wherein the nucleotide sequence is nucleotides 84 through 1694 SEQ ID NO:1.

10. An isolated genomic nucleic acid corresponding to the nucleic acid of claim 5.

11. An isolated genomic nucleic acid corresponding to the nucleic acid of claim 6.

12. An isolated nucleic acid comprising a nucleotide sequence that encodes a polypeptide having cation exchange activity, said polypeptide comprising amino acids 437 through 441 of SEQ ID NO:2.

13. An isolated nucleic acid of claim 12 that shares nucleotide sequence identity with the nucleotide sequences of the nucleic acids of claim 5, wherein the percent nucleotide sequence identity is selected from the group consisting of: at least 85%, at least 90%, at least 95%, at least 97.5%, at least 99%, and at least 99.5%.

14. An expression vector, comprising at least one nucleic acid according to claim 5.

15. A recombinant host cell, comprising at least one nucleic acid according to claim 5.

16. The recombinant host cell of claim 15, wherein the nucleic acid is integrated into the host cell genome.

17. A process for producing a polypeptide encoded by the nucleic acid of claim 5, comprising culturing a recombinant host cell under conditions promoting expression of said polypeptide, wherein the recombinant host cell comprises at least one nucleic acid according to claim 5.

18. The process of claim 17, further comprising purifying said polypeptide.

19. The polypeptide produced by the process of claim 18.

20. An isolated antibody that binds to the polypeptide of claim 1.

21. An isolated antibody that binds to the polypeptide of claim 2.

22. The antibody of claim 20, wherein the antibody is a monoclonal antibody.

23. The antibody of claim 20, wherein the antibody is a human antibody.

24. The antibody of claim 20, wherein the antibody is a humanized antibody.

25. The antibody of claim 21, wherein the antibody is a monoclonal antibody.

26. The antibody of claim 21, wherein the antibody is a human antibody.

27. The antibody of claim 21, wherein the antibody is a humanized antibody.

28. The antibody of claim 20, wherein the antibody inhibits the activity of the polypeptide of claim 1.

29. A method of designing an inhibitor of the polypeptide of claim 1, the method comprising the steps of determining the three-dimensional structure of such polypeptide, analyzing the three-dimensional structure for the likely binding sites of substrates, synthesizing a molecule that incorporates a predicted reactive site, and determining the polypeptide-inhibiting activity of the molecule.

30. A method for identifying compounds that alter RIPPA polypeptide activity comprising:
(a) mixing a test compound with the polypeptide of claim 1; and
(b) determining whether the test compound alters the RIPPA polypeptide activity of said polypeptide.

31. A method for identifying compounds that inhibit the binding activity of RIPPA polypeptides comprising:
(a) mixing a test compound with the polypeptide of claim 1 and a binding partner of said polypeptide; and
(b) determining whether the test compound inhibits the binding activity of said polypeptide.

32. A method for increasing cation exchange activity comprising providing at least one compound selected from the group consisting of the polypeptide of claim 1 and agonists of said polypeptides.

33. The method of claim 32, wherein the method comprises increasing cation exchange activity in a patient by administering to said patient at least one compound selected from the group consisting of the polypeptide of claim 1 and agonists of said polypeptides.

34. A method for decreasing cation exchange activity comprising providing at least one antagonist of the polypeptide of claim 1.

35. The method of claim 34, wherein the method comprises decreasing cation exchange activity in a patient by administering at least one antagonist of the polypeptide of claim 1 to said patient.

36. The method of claim 34, wherein the antagonist is an antibody that inhibits the activity of said polypeptide.

37. A method for treating a subject suffering from one or more forms of osteopenia, comprising administering to said subject an antagoinst to one or more polypeptides of claim 1.

38. The method of claim 37, wherein the osteopenia is selected from the group consisting of osteoporosis, osteomyelitis, hypercalcemia, osteopenia brought on by surgery or steroid administration, prosthetic loosening, Paget's disease, osteonecrosis, bone loss due to rheumatoid arthritis, periodontal bone loss, and cancers that may metastasize to bone and induce bone breakdown, such as multiple myeloma, breast cancer and some melanomas.

39. A method for treating a subject suffering from one or more forms of osteopetrosis, comprising administering an agonist of the polypeptide of claim 1.

40. A method of screening antagonists and/or agonists of the polypeptide of claim 1, comprising:
(a) culturing cells in the presence of bone, dentin or calcium phosphate matrix;
(b) exposing the cells to at least one agonist or antagonist of RIPPA or RIPPA-like polypeptides; and
(c) determining the number and/or size of resorption lacunae formed in the bone, dentin or calcium phosphate matrix, wherein a difference in the number and/or size of resorption lacunae relative to cells not exposed to the agonist or antagonist indicates a therapeutic agent.


SEQUENCE LISTING (13 sequences)

SEQ ID NO: 1 - DNA, 1768 bp, Homo sapiens
cgttcagacggagcctggtcgacttcgccggagactgccagatctcgttcctcttccctgtgtcatcttcttaattataaataatgggggatgaagataaaagaattacatatgaagattcagaaccatccacaggaatgaattacacgccctccatgcatcaagaagcacaggaggagacagttatgaagctcaaaggtatagatgcaaatgaaccaacagaaggaagtattcttttgaaaagcagtgaaaaaaagctacaagaaacaccaactgaagcaaatcacgtacaaagactgagacaaatgctggcttgccctccacatggtttactggacagggtcataacaaatgttaccatcattgttcttctgtgggctgtagtttggtcaattactggcagtgaatgtcttcctggaggaaacctatttggaattataatcctattctattgtgccatcattggtggtaaacttttggggcttattaagttacctacattgcctccactgccttctcttcttggcatgctgcttgcagggtttctcatcagaaatatcccagtcatcaacgataatgtgcagatcaagcacaagtggtcttcctctttgagaagcatagccctgtctatcattctggttcgtgctggccttggtctggattcaaaggccctgaagaagttaaagggcgtttgtgtaagactgtccatgggtccctgtattgtggaggcgtgcacatctgctcttcttgcccattacctgctgggtttaccatggcaatggggatttatactgggttttgttttaggtgctgtatctccagctgttgtggtgccttcaatgctccttttgcagggaggaggctatggtgttgagaagggtgtcccaaccttgctcatggcagctggcagcttcgatgacattctggccatcactggcttcaacacatgcttgggcatagccttttccacaggctctactgtctttaatgtcctcagaggagttttggaggtggtaattggtgtggcaactggatctgttcttggatttttcattcagtactttccaagccgtgaccaggacaaacttgtgtgtaagagaacattccttgtgttggggttgtctgtgctagctgtgttcagcagtgtgcattttggtttccctggatcaggaggactgtgcacgttggtcatggctttccttgcaggcatgggatggaccagcgaaaaggcagaggttgaaaagataattgcagttgcctgggacatttttcagccccttctttttggactaattggagcagaggtatctattgcatctctcagaccagaaactgtaggcctttgtgttgccaccgtaggcattgcagtattgatacgaattttgactacatttctgatggtgtgttttgctggttttaacttaaaagaaaagatatttatttcttttgcatggcttccaaaggccacagttcaggctgcaataggatctgtggctttggacacagcaaggtcacatggagagaaacaattagaagactatggaatggatgtgttgacagtggcatttttgtccatcctcatcacagccccaattggaagtctgcttattggtttactgggccccaggcttctgcagaaagttgaacatcaaaataaagatgaagaagttcaaggagagacttctgtgcaagtttagaggaagcgcggattctattactggaaactttgggactgaaaggccaaagcttctgggcccaccatcaacgc

SEQ ID NO: 2 - protein, 537 aa, Homo sapiens
MGDEDKRITYEDSEPSTGMNYTPSMHQEAQEETVMKLKGIDANEPTEGSILLKSSEKKLQETPTEANHVQRLRQMLACPPHGLLDRVITNVTIIVLLWAVVWSITGSECLPGGNLFGIIILFYCAIIGGKLLGLIKLPTLPPLPSLLGMLLAGFLIRNIPVINDNVQIKHKWSSSLRSIALSIILVRAGLGLDSKALKKLKGVCVRLSMGPCIVEACTSALLAHYLLGLPWQWGFILGFVLGAVSPAVVVPSMLLLQGGGYGVEKGVPTLLMAAGSFDDILAITGFNTCLGIAFSTGSTVFNVLRGVLEVVIGVATGSVLGFFIQYFPSRDQDKLVCKRTFLVLGLSVLAVFSSVHFGFPGSGGLCTLVMAFLAGMGWTSEKAEVEKIIAVAWDIFQPLLFGLIGAEVSIASLRPETVGLCVATVGIAVLIRILTTFLMVCFAGFNLKEKIFISFAWLPKATVQAAIGSVALDTARSHGEKQLEDYGMDVLTVAFLSILITAPIGSLLIGLLGPRLLQKVEHQNKDEEVQGETSVQV

SEQ ID NO: 3 - protein, 480 aa, Homo sapiens
MGDEDKRITYEDSEPSTGMNYTPSMHQEAQEETVMKLKGIDANEPTEGSILLKSSEKKLQETPTEANHVQRLRQMLACPPHGLLDRVITNGMLLAGFLIRNIPVINDNVQIKHKWSSSLRSIALSIILVRAGLGLDSKALKKLKGVCVRLSMGPCIVEACTSALLAHYLLGLPWQWGFILGFVLGAVSPAVVVPSMLLLQGGGYGVEKGVPTLLMAAGSFDDILAITGFNTCLGIAFSTGSTVFNVLRGVLEVVIGVATRSVLGFFIQYFPSRDQDKLVCKRTFLVLGLSVLAVFSSVHFGFPGSGGLCTLVMAFLAGMGWTSEKAEVEKIIAVAWDIFQPLLFGLIGAEVSIASLRPETVGLCVATVGIAVLIRILTTFLMVCFAGFNLKEKIFISFAWLPKATVQAAIGSVALDTARSHGEKQLEDYGMDVLTVAFLSILITAPIGSLLIGLLGPRLLQKVEHQNKDEEVQGETSVQV

SEQ ID NO: 4 - protein, 475 aa, Homo sapiens
MGDEDKRITYEDSEPSTGMNYTPSMHQEAQEETVMKLKGIDANEPTEGSILLKSSEKKLQETPTEANHVQRLRQMLACPPHGLLDRVITNVTIIVLLWAVVWSITGSECLPGGNLFGIIILFYCAIIGGKLLGLIKLPTLPPLPSLLGMLLAGFLIRNIPVINDNVQIKHKWSSSLRSIALSIILVRAGLGLDSKGGGYGVEKGVPTLLMAAGSFDDILAITGFNTCLGIAFSTGSTVFNVLRGVLEVVIGVATGSVLGFFIQYFPSRDQDKLVCKRTFLVLGLSVLAVFSSVHFGFPGSGGLCTLVMAFLAGMGWTSEKAEVEKIIAVAWDIFQPLLFGLIGAEVSIASLRPETVGLCVATVGIAVLIRILTTFLMVCFAGFNLKEKIFISFAWLPKATVQAAIGSVALDTARSHGEKQLEDYGMDVLTVAFLSILITAPIGSLLIGLLGPRLLQKVEHQNKDEEVQGETSVQV

SEQ ID NO: 5 - protein, 418 aa, Homo sapiens
MGDEDKRITYEDSEPSTGMNYTPSMHQEAQEETVMKLKGIDANEPTEGSILLKSSEKKLQETPTEANHVQRLRQMLACPPHGLLDRVITNGMLLAGFLIRNIPVINDNVQIKHKWSSSLRSIALSIILVRAGLGLDSKGGGYGVEKGVPTLLMAAGSFDDILAITGFNTCLGIAFSTGSTVFNVLRGVLEVVIGVATGSVLGFFIQYFPSRDQDKLVCKRTFLVLGLSVLAVFSSVHFGFPGSGGLCTLVMAFLAGMGWTSEKAEVEKIIAVAWDIFQPLLFGLIGAEVSIASLRPETVGLCVATVGIAVLIRILTTFLMVCFAGFNLKEKIFISFAWLPKATVQAAIGSVALDTARSHGEKQLEDYGMDVLTVAFLSILITAPIGSLLIGLLGPRLLQKVEHQNKDEEVQGETSVQV

SEQ ID NO: 6 - DNA, 1644 bp, Mus musculus
atggaggatgaagataagacagctgaatgtcagcattcaaagccacctacggggatcacacacgaggctcctccacatcacgaactacaggaagagagagtcatgagcctcagaggtacagacagaagtgaaccgacagaaggcagtaacctgctgaccagcggtgaaaaaaagccacaggactcaccaacagaacccaatggcctgcagagtctgaggcgattcctggcctgccctccacgaggctgcctggcaagagtgataacgaacggtaccatggttgttcttctgtgggccatggtttggtcagttaccggccctgaatgtcttcctggaggaaatctgtttggaattatcattctgttctattgttccatcaccggaggtaaactttttggactcattaagtttccaacattgcctcctctgcctcctcttcttggcatgctgctagctgggtttctcttgaggaatatcccagtcatcaatgatagcgtccggatccaacacaagtggtcgtcatctttgagaagcatagccctttctgtcattctggttcgtgctggccttggtctagattcaaaggccctgaggaagctgaagggtgtgtgtgtgcgactggccatgggtccctgcatcgtggaggcgtgtgcttctgcgattctctcacacttcctgatggggttgccatggcaatgggggttcatcctgggttttgtcgtaggtgccgtgtccccagctgtcgtggtgccctccatgctccttttgcaggaaggaggctacggtgttggaaaaggtatcccaaccttactcatggccgccggcagcttcgatgacatcctggccatcactggcttcaacacgtgcttaggcgtggccttttccacaggatctacagtttttaacatcttcagaggcatcttggaggtggtaattggtgtggcagctggatcttttcttgggttttttatccagtacttcccgagcagggaccaggacaacctcgtgtggaagcgagcctttctggttctgggttttgctgtgctcgctgtgttcagcagtgtgtattttagcttcccggggtctggaggactctgcacgttggtcatggctttcctagcaggcatgaggtggactgacaagaagtcagaggtagaaaaggtcattgcagttacctgggacgttttccagcctcttctttttggcctgattggagcagaggtttccattgtgtctctcagagcagaaacggttggcctttgtgttgcaaccctcagcatcgcagtgcttatacgaattctgactacattcctgatggtgtgtttcgctggctttaacataaaggaaaagatatttatttcttttgcctggcttccaaaggccacggtccaggctgccattggctctgtggctctggacacggcaagatcccacggagagaagcagctggaagactatgggatggatgtgctgacggtggcatttttggccatcctcattacagcaccaattggaagcctactgattggtttgctgggtcccagggttcttcagaaatctgaacatcgaaccgaagaggaggttcaaggagagacttctgcacacattcagaggaagcctgaggattccattacggaagcctga

SEQ ID NO: 7 - protein, 547 aa, Mus musculus
MEDEDKTAECQHSKPPTGITHEAPPHHELQEERVMSLRGTDRSEPTEGSNLLTSGEKKPQDSPTEPNGLQSLRRFLACPPRGCLARVITNGTMVVLLWAMVWSVTGPECLPGGNLFGIIILFYCSITGGKLFGLIKFPTLPPLPPLLGMLLAGFLLRNIPVINDSVRIQHKWSSSLRSIALSVILVRAGLGLDSKALRKLKGVCVRLAMGPCIVEACASAILSHFLMGLPWQWGFILGFVVGAVSPAVVVPSMLLLQEGGYGVGKGIPTLLMAAGSFDDILAITGFNTCLGVAFSTGSTVFNIFRGILEVVIGVAAGSFLGFFIQYFPSRDQDNLVWKRAFLVLGFAVLAVFSSVYFSFPGSGGLCTLVMAFLAGMRWTDKKSEVEKVIAVTWDVFQPLLFGLIGAEVSIVSLRAETVGLCVATLSIAVLIRILTTFLMVCFAGFNIKEKIFISFAWLPKATVQAAIGSVALDTARSHGEKQLEDYGMDVLTVAFLAILITAPIGSLLIGLLGPRVLQKSEHRTEEEVQGETSAHIQRKPEDSITEA

SEQ ID NO: 8 - protein, 550 aa, Drosophila melanogaster
MGHNGGGSSNGGANYVEESWMYNFCLKCRGEEPSASWEPPFWQKIFPYPLCPTFRTVSRLISIILIGVLIWVTAFVIIGDSAAPGGQLFGLVVLTVAANFGGYLISLTTLPRLIGMLLVGILFQNVGWADIDGDFSKVTAHLRKFALTIILTRAGLEMEPEAFKKVYKTILKLGIIPWCVEAVVMAVMSHFLLDLPWIWAFLLGSIIAAVSPAVVVPCLFRLRTKGYGVAKGIPTLVVAVAGVDDALSVAIFGIISTVMFSDKGLAYQIAQAPVCILGGLGFGVVWGSLARIFPEKGDAYVVPLRTLLLFTGGLMAIYGSEELGFEGAGPLAVVFSAFVSNLFWCKDGWDVEDNPVSTAFEIFWMIFEPILFGLTGATIKIRELDSHTVSIGAACIFTGAILRILTTAGIAFGDRLNTKEKFFVGLSWMAKATVQAALGPVALKHLGDDATEEERNWANIVQTICVFSIVLTAPLGAIMISVTGTKLLTKTKQPQDLSGWRRSHRPSIRDISIIDEEEEREDPEIPEDKETHDNTLNNAHIPSISYTYNT

SEQ ID NO: 9 - protein, 399 aa, Methanothermobacter thermautotrophicus
MEYVAFSVAVIILLGLLFNRVFTRARLPGILGMLILGILIGPHGLNLISKSIMDISPDLRVIALIIILLRAGFGINLESLRKVGMTAVKMSFIPDVFEGLAVMVASHYILGLPIIEAGILGFVIAAVSPAVIVPQMLSFIERRMGTAKGIPTIILTGASVDDVVSITLFSVFLGIYQGQQVNLLVEAIGVPLSILTGITGGLALGLALVYLFRRFEIRNTEKTLIILGAAILLKNMGDLISTYLPIAALVGVMVVGLVILERMPETGLKLSEKFNKVWIFAEILLFVLVGAQVDVRLIFHVGLLGLAVITAGLMVRSIGVLLSLKGTDLNLKEKVFCISAYIPKATVQAAIGAIPLAAGVAAGETILAMAVLAILFTAPLGSLAVKWTGERFLEVERGN

SEQ ID NO: 10 - DNA, 19 bp, Artificial Sequence
  Oligonucleotide primer
ggttggcctttgtgttgca

SEQ ID NO: 11 - DNA, 20 bp, Artificial Sequence
  Oligonucleotide primer
aagccagcgaaacacaccat

SEQ ID NO: 12 - DNA, 20 bp, Artificial Sequence
  Oligonucleotide primer
gtcccagcgtcgtgattagc

SEQ ID NO: 13 - DNA, 21 bp, Artificial Sequence
  Oligonucleotide primer
ttccaaatcctcggcataatg